SEQUENCE LISTING (28 sequences)

SEQ ID NO: 1 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 2 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 3 - protein, 354 aa, Homo sapiens
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTSPRSHRSDGEFSPHSHYSDSDEAS

SEQ ID NO: 4 - DNA, 2379 bp, Homo sapiens
  source 1..2379: mol_type = genomic DNA
gacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaaaaaaaaaaaaaaa

SEQ ID NO: 5 - DNA, 9309 bp, Homo sapiens
  source 1..9309: mol_type = genomic DNA
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaatgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccgggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgccgctgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttctactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgccacgggggcacctgccaggaccgcgacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacatcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaatgagtgtgaatccaacccttgtgtcaacggcggcacctgcaaagacatgaccagtggctacgtgtgcacctgccgggagggcttcagcggtcccaactgccagaccaacatcaacgagtgtgcgtccaacccatgtctgaaccagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacgggctggcaagggcagacctgtgaggtcgacatcaacgagtgcgttctgagcccgtgccggcacggcgcatcctgccagaacacccacggcggctaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgatgtcaatgagtgcgactcacagccctgcctgcatggcggcacctgtcaggacggctgcggctcctacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaaacggcgcgccccacgagaccccggcttcctttcccaagccttcgggcgtctgtgtgcgctctgtggatgccagggccgaccagaggagcctttttaaaacacatgtttttatacaaaataagaacgaggattttaattttttttagtatttatttatgtacttttattttacacagaaacactgcctttttatttatatgtactgttttatctggccccaggtagaaacttttatctattctgagaaaacaagcaagttctgagagccagggttttcctacgtaggatgaaaagattcttctgtgtttataaaatataaacaaagattcatgatttataaatgccatttatttattgattccttttttcaaaatccaaaaagaaatgatgttggagaagggaagttgaacgagcatagtccaaaaagctcctggggcgtccaggccgcgccctttccccgacgcccacccaaccccaagccagcccggccgctccaccagcatcacctgcctgttaggagaagctgcatccagaggcaaacggaggcaaagctggctcaccttccgcacgcggattaatttgcatctgaaataggaaacaagtgaaagcatatgggttagatgttgccatgtgttttagatggtttcttgcaagcatgcttgtgaaaatgtgttctcggagtgtgtatgccaagagtgcacccatggtaccaatcatgaatctttgtttcaggttcagtattatgtagttgttcgttggttatacaagttcttggtccctccagaaccaccccggccccctgcccgttcttgaaatgtaggcatcatgcatgtcaaacatgagatgtgtggactgtggcacttgcctgggtcacacacggaggcatcctacccttttctggggaaagacactgcctgggctgaccccggtggcggccccagcacctcagcctgcacagtgtcccccaggttccgaagaagatgctccagcaacacagcctgggccccagctcgcgggacccgaccccccgtgggctcccgtgttttgtaggagacttgccagagccgggcacattgagctgtgcaacgccgtgggctgcgtcctttggtcctgtccccgcagccctggcagggggcatgcggtcgggcaggggctggagggaggcgggggctgcccttgggccacccctcctagtttgggaggagcagatttttgcaataccaagtatagcctatggcagaaaaaatgtctgtaaatatgtttttaaaggtggattttgtttaaaaaatcttaatgaatgagtctgttgtgtgtcatgccagtgagggacgtcagacttggctcagctcggggagccttagccgcccatgcactggggacgctccgctgccgtgccgcctgcactcctcagggcagcctcccccggctctacgggggccgcgtggtgccatccccagggggcatgaccagatgcgtcccaagatgttgatttttactgtgttttataaaatagagtgtagtttacagaaaaagactttaaaagtgatctacatgaggaactgtagatgatgtatttttttcatcttttttgttaactgatttgcaataaaaatgatactgatggtgaaaaaaaaaaaaaaa

SEQ ID NO: 6 - DNA, 1065 bp, Homo sapiens
  source 1..1065: mol_type = genomic DNA
atgtcccgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagttag

SEQ ID NO: 7 - protein, 802 aa, Homo sapiens
VLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 8 - DNA, 2406 bp, Homo sapiens
  source 1..2406: mol_type = genomic DNA
gtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaag

SEQ ID NO: 9 - protein, 440 aa, synthetic construct
  VARIANT 6: note = Ser or Asn
  VARIANT 8: note = Thr or Ala
  VARIANT 24: note = Tyr or Ile
  VARIANT 33: note = Gln or His
  VARIANT 78: note = Thr or Ala
  VARIANT 79: note = Pro or Thr
  SITE 80: note = May or may not be present
  VARIANT 83: note = Leu or Pro
  VARIANT 85: note = Gly or Glu
  VARIANT 87: note = Asn or Asp
  VARIANT 93: note = Ser or Asn
  VARIANT 103: note = Val or Met
  VARIANT 161: note = Gly or Thr
  VARIANT 163: note = Pro or Leu
  VARIANT 164: note = Asn or Ser
  VARIANT 183: note = Ser or Thr
  VARIANT 210: note = Pro, Ala or Thr
  VARIANT 212: note = Gln or Ser
  VARIANT 215: note = Ser or Thr
  VARIANT 219: note = Pro or Ser
  SITE 228: note = May or may not be present
  VARIANT 233: note = Gln or Arg
  VARIANT 234: note = Ala or Gly
  VARIANT 235: note = Ser or Thr
  VARIANT 258: note = Glu or Asp
  VARIANT 273: note = Ala, Thr or Pro
  VARIANT 275: note = Gly or Ala
  VARIANT 283: note = Pro or Ser
  VARIANT 284: note = Pro or Ser
  VARIANT 285: note = Ala or Ser
  VARIANT 326: note = Val or Ala
  VARIANT 331: note = Val or Gly
  VARIANT 335: note = Lys or Arg
  VARIANT 344: note = Thr or Ser
  VARIANT 355: note = Asp or Val
  VARIANT 407: note = Val or Ile
  VARIANT 410: note = Glu or Asp
  VARIANT 412: note = Gln or His
  VARIANT 415: note = Thr or Ile
  VARIANT 418: note = Glu or Lys
  VARIANT 439: note = Cys or Gly
MPLNVXFXNRNYDLDYDSVQPYFXCDEEENFYXQQQQSELQPPAPSEDIWKKFELLPTPPLSPSRRSGLCSPSYVAVXXSFSXRXDXDGGGGXFSTADQLEMXTELLGGDMVNQSFICDPDDETFIKNIIIQDCMWSGFSAAAKLVSEKLASYQAARKDSXSXXPARGHSVCSTSSLYLQDLXAAASECIDPSVVFPYPLNDSSSPKSCXSXDSXAFSXSSDSLLSSTESSPXXXPEPLVLHEETPPTTSSDSEEEQXDEEEIDVVSVEKRQXPXKRSESGSXXXGGHSKPPHSPLVLKRCHVSTHQHNYAAPPSTRKDYPAAKRXKLDSXRVLXQISNNRKCXSPRSSDTEENXKRRTHNVLERQRRNELKRSFFALRDQIPELENNEKAPKVVILKKATAYILSXQAXEXKLXSEXDLLRKRREQLKHKLEQLRNSXA

SEQ ID NO: 10 - protein, 803 aa, synthetic construct
  VARIANT 82: note = Asp or Ser
  VARIANT 102: note = Met or Val
  VARIANT 228: note = Leu or Ile
  VARIANT 307: note = Lys or Arg
  VARIANT 374: note = Thr or Ala
  VARIANT 375: note = Ala or Pro
  VARIANT 385: note = Thr or Pro
  VARIANT 395: note = Ser or Asn
  VARIANT 398: note = Ser or Pro
  VARIANT 399: note = Ala or Gly
  VARIANT 400: note = Thr or Val
  VARIANT 405: note = Ala or Val
  VARIANT 410: note = Thr or Ser
  VARIANT 416: note = Gly or Ser
  VARIANT 442: note = Gly or Ser
  VARIANT 476: note = Met or Val
  VARIANT 479: note = Ser or Asn
  VARIANT 492: note = Ser or Gly
  VARIANT 506: note = Ala or Gly
  VARIANT 509: note = Ser or Gly
  VARIANT 515: note = Pro or Thr
  VARIANT 516: note = Pro or Gly
  VARIANT 528: note = Ser or Gly
  VARIANT 529: note = Ala or Thr
  VARIANT 534: note = Ser or Gly
  VARIANT 535: note = Thr or Ser
  VARIANT 536: note = Asn or Ser
  VARIANT 537: note = Gly or Ser
  VARIANT 538: note = Thr or Gly
  VARIANT 541: note = Met or Leu
  VARIANT 547: note = Ala or Gly
  VARIANT 548: note = Pro or Ser
  VARIANT 549: note = Ala or Thr
  VARIANT 559: note = Pro or Ser
  VARIANT 563: note = Asn or Ser
  VARIANT 568: note = Ser or Asn
  VARIANT 575: note = Pro or Gly
  VARIANT 576: note = Gly or Ser
  VARIANT 578: note = Thr or Ala
  VARIANT 581: note = Thr or Pro
  VARIANT 587: note = Ala or Pro
  VARIANT 588: note = Gly or Ser
  VARIANT 592: note = Gly or Ser
  VARIANT 594: note = Met or Val
  VARIANT 597: note = Leu or Ile
  VARIANT 602: note = Ser or Ala
  VARIANT 603: note = Thr or Ala
  VARIANT 604: note = Asn or Ser
  VARIANT 605: note = Thr or Ala
  VARIANT 608: note = Pro or Gln
  VARIANT 609: note = Met or Ile
  VARIANT 610: note = Met or Ile
  SITE 611: note = May or may not be present
  VARIANT 617: note = Ser or Asn
  VARIANT 639: note = Leu, Ile or Met
  VARIANT 641: note = Gln or Pro
  SITE 646..659: note = May or may not be present
  VARIANT 662: note = Pro or Ser
  VARIANT 667: note = Ser or Gly
  VARIANT 673: note = Asn or Ser
  VARIANT 699: note = Pro or Ala
  VARIANT 709: note = Gln or Pro
  VARIANT 719: note = Met or Leu
  VARIANT 723: note = Met or Val
  VARIANT 725: note = Thr or Ala
  VARIANT 726: note = Thr or Ala
  SITE 740: note = May or may not be present
  VARIANT 773: note = Pro or Arg
  VARIANT 777: note = Ile or Val
  VARIANT 784: note = Ile or Val
  VARIANT 790: note = Thr or Ser
  VARIANT 792: note = Pro or Gln
  VARIANT 796: note = Thr or Ala
  VARIANT 797: note = His or Arg
VLLSRKRRRQHGQLWFPEGFKVSEASKKKRREPLGEDSVGLKPLKNASDGALMDDNQNEWGDEDLETKKFRFEEPVVLPDLXDQTDHRQWTQQHLDAADLRXSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILXRNRATDLDARMHDGTTPLILAARLAVEGMLEDLINSHADVNAVDDLGKSALHWAAAVNNVDAAVVLLKNGANKDMQNNXEETPLFLAAREGSYETAKVLLDHFANRDITDHMDRLPRDIAQERMHHDIVRLLDEYNLVRSPQLHGXXLGGTPTLSPXLCSPNGYLGXLKXXXQGKKXRKPSXKGLACXSKEAKDLKARRKKSQDGKGCLLDSSXMLSPVDSLESPHGYLSDVASPPLLPSPFQQSPSXPLXHLPGMPDTHLGIXHLNVAAKPEMAALXGGXRLAFEXXPPRLSHLPVASXXSTVLXXXXXGAXNFTVGXXXSLNGQCEWLXRLQXGMVPXQYNPLRXXVXPGXLSTQAXXLQHXMXGPXHSSLXXXXLSXXXSYQGLPXTRLATQPHLVQTQQVQPQNLQXQXQNLQPANIQQQQSLQPPPPPXQPHLXVSSAAXGHLGRSFLSGEPSQADVQPLGPSSLXVHTILPQESXALPTSLPSSXVPPXTXXQFLTPPSQHSYSSSPVDNTPSHQLQVPEHPFLTPSPESPDQWSSSSXHSNXSDWSEGXSSPPTXMXSQIXXIPEAFK

SEQ ID NO: 11 - protein, 354 aa, synthetic construct
  VARIANT 22: note = Gln or His
  VARIANT 28: note = Leu or Val
  SITE 30..31: note = May or may not be present
  SITE 34..35: note = May or may not be present
  VARIANT 45: note = Glu or Asp
  VARIANT 46: note = His or Leu
  VARIANT 51: note = Pro or Ala
  VARIANT 66: note = Ala or Thr
  VARIANT 72: note = Ile or Leu
  VARIANT 88: note = Ser or Gly
  VARIANT 99: note = Val or Ala
  VARIANT 101: note = Gly or Ser
  VARIANT 102: note = Arg or Gln
  VARIANT 110: note = Ser or Gly
  VARIANT 111: note = Gly or Cys
  VARIANT 113: note = Ala or Gly
  SITE 114: note = May or may not be present
  VARIANT 115: note = Ser or Leu
  VARIANT 219: note = Ser or Asn
  VARIANT 220: note = Gly or Val
  VARIANT 228: note = Pro or Thr
  VARIANT 233: note = Ser or Asn
  VARIANT 242: note = Ala or Ser
  VARIANT 250: note = Ala or Asn
  VARIANT 251: note = Ala or Ser
  VARIANT 252: note = Thr or Ala
  VARIANT 253: note = Ala or Val
  VARIANT 258: note = Gln or Pro
  VARIANT 260: note = Ser or Pro
  VARIANT 263: note = Ser or Gly
  VARIANT 264: note = Gln or Pro
  VARIANT 271: note = Ser or Pro
  VARIANT 278: note = Ala or Gly
  VARIANT 282: note = Ala or Ser
  VARIANT 295: note = Ser or Pro
  VARIANT 296: note = Thr or Ala
  VARIANT 300: note = Ser or Arg
  VARIANT 310: note = Asn or Asp
  VARIANT 318: note = Ser or Gly
  VARIANT 326: note = Glu or Asp
MSRLLHAEEWAEVKELGDHHRXPQPHHXPQPPPPPQPPATLQARXXPVYPXELSLLDSTDPRAWLXPTLQGXCTARAAQYLLHSPELXASEAAAPRDEXDXXGELVRRSXXGXSXSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPXXGEQPPPPXASCKXDHHHLRTAXSYEGGAGXXXXAGAQXAXGGXXRPTPPGXCRTRFSXPASXGGYSVQLDALHFXXFEDXALTAMMAQKXLSPSLPGXILQPVQEXNSKTSPRSHRSDGEFSPHSHYSDSDEAS

SEQ ID NO: 12 - protein, 462 aa, Homo sapiens
MPSCSTSTMPGMICKNPDLEFDSLQPCFYPDEDDFYFGGPDSTPPGEDIWKKFELLPTPPLSPSRGFAEHSSEPPSWVTEMLLENELWGSPAEEDAFGLGGLGGLTPNPVILQDCMWSGFSAREKLERAVSEKLQHGRGPPTAGSTAQSPGAGAASPAGRGHGGAAGAGRAGAALPAELAHPAAECVDPAVVFPFPVNKREPAPVPAAPASAPAAGPAVASGAGIAAPAGAPGVAPPRPGGRQTSGGDHKALSTSGEDTLSDSDDEDDEEEDEEEEIDVVTVEKRRSSSNTKAVTTFTITVRPKNAALGPGRAQSSELILKRCLPIHQQHNYAAPSPYVESEDAPPQKKIKSEASPRPLKSVIPPKAKSLSPRNSDSEDSERRRNHNILERQRRNDLRSSFLTLRDHVPELVKNEKAAKVLKKATEYVHSLQAEEHQLLLEKEKLQARQQQLLKKIEHARTC

SEQ ID NO: 13 - DNA, 2613 bp, Homo sapiens
  source 1..2613: mol_type = genomic DNA
gtcatctgtctggacgcgctgggtggatgcggggggctcctgggaactgtgttggagccgagcaagcgctagccaggcgcaagcgcgcacagactgtagccatccgaggacacccccgcccccccggcccacccggagacacccgcgcagaatcgcctccggatcccctgcagtcggcgggagtgttggaggtcggcgccggcccccgccttccgcgccccccacgggaaggaagcacccccggtattaaaacgaacggggcggaaagaagccctcagtcgccggccgggaggcgagccgatgccgagctgctccacgtccaccatgccgggcatgatctgcaagaacccagacctcgagtttgactcgctacagccctgcttctacccggacgaagatgacttctacttcggcggccccgactcgacccccccgggggaggacatctggaagaagtttgagctgctgcccacgcccccgctgtcgcccagccgtggcttcgcggagcacagctccgagcccccgagctgggtcacggagatgctgcttgagaacgagctgtggggcagcccggccgaggaggacgcgttcggcctggggggactgggtggcctcacccccaacccggtcatcctccaggactgcatgtggagcggcttctccgcccgcgagaagctggagcgcgccgtgagcgagaagctgcagcacggccgcgggccgccaaccgccggttccaccgcccagtccccgggagccggcgccgccagccctgcgggtcgcgggcacggcggggctgcgggagccggccgcgccggggccgccctgcccgccgagctcgcccacccggccgccgagtgcgtggatcccgccgtggtcttcccctttcccgtgaacaagcgcgagccagcgcccgtgcccgcagccccggccagtgccccggcggcgggccctgcggtcgcctcgggggcgggtattgccgccccagccggggccccgggggtcgcccctccgcgcccaggcggccgccagaccagcggcggcgaccacaaggccctcagtacctccggagaggacaccctgagcgattcagatgatgaagatgatgaagaggaagatgaagaggaagaaatcgacgtggtcactgtggagaagcggcgttcctcctccaacaccaaggctgtcaccacattcaccatcactgtgcgtcccaagaacgcagccctgggtcccgggagggctcagtccagcgagctgatcctcaaacgatgccttcccatccaccagcagcacaactatgccgccccctctccctacgtggagagtgaggatgcacccccacagaagaagataaagagcgaggcgtccccacgtccgctcaagagtgtcatccccccaaaggctaagagcttgagcccccgaaactctgactcggaggacagtgagcgtcgcagaaaccacaacatcctggagcgccagcgccgcaacgaccttcggtccagctttctcacgctcagggaccacgtgccggagttggtaaagaatgagaaggccgccaaggtggtcattttgaaaaaggccactgagtatgtccactccctccaggccgaggagcaccagcttttgctggaaaaggaaaaattgcaggcaagacagcagcagttgctaaagaaaattgaacacgctcggacttgctagacgcttctcaaaactggacagtcactgccactttgcacattttgattttttttttaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttctttttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggaagttcatacctaagtactgtaataatacctcaatgtttgaggagcatgttttgtatacaaatatattgttaatctctgttatgtactgtactaattcttacactgcctgtatactttagtatgacgctgatacataactaaatttgatacttatattttcgtatgaaaatgagttgtgaaagttttgagtagatattactttatcactttttgaactaagaaacttttgtaaagaaatttactatatatatatgcctttttcctagcctgtttcttcctgttaatgtatttgttcatgtttggtgcatagaactgggtaaatgcaaagttctgtgtttaatttcttcaaaatgtatatatttagtgctgcatcttatagcactttgaaatacctcatgtttatgaaaataaatagcttaaaattaaatgaaaaaaaaa

SEQ ID NO: 14 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 15 - DNA, 11474 bp, Homo sapiens
  source 1..11474: mol_type = genomic DNA
gcttgcggtgggaggaggcggctgaggcggaaggacacacgaggctgcttcgttgcacacccgagaaagtttcagccaaacttcgggcggcggctgaggcggcggccgaggagcggcggactcggggcgcggggagtcgaggcatttgcgcctgggcttcggagcgtagcgccagggcctgagcctttgaagcaggaggaggggaggagagagtggggctcctctatcgggaccccctccccatgtggatctgcccaggcggcggcggcggcggcggaggaggaggcgaccgagaagatgcccgccctgcgccccgctctgctgtgggcgctgctggcgctctggctgtgctgcgcggcccccgcgcatgcattgcagtgtcgagatggctatgaaccctgtgtaaatgaaggaatgtgtgttacctaccacaatggcacaggatactgcaaatgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcaacatctcatccatgctttgtgtctcgaccctgcctgaatggcggcacatgccatatgctcagccgggatacctatgagtgcacctgtcaagtcgggtttacaggtaaggagtgccaatggacggatgcctgcctgtctcatccctgtgcaaatggaagtacctgtaccactgtggccaaccagttctcctgcaaatgcctcacaggcttcacagggcagaaatgtgagactgatgtcaatgagtgtgacattccaggacactgccagcatggtggcacctgcctcaacctgcctggttcctaccagtgccagtgccctcagggcttcacaggccagtactgtgacagcctgtatgtgccctgtgcaccctcaccttgtgtcaatggaggcacctgtcggcagactggtgacttcacttttgagtgcaactgccttccaggttttgaagggagcacctgtgagaggaatattgatgactgccctaaccacaggtgtcagaatggaggggtttgtgtggatggggtcaacacttacaactgccgctgtcccccacaatggacaggacagttctgcacagaggatgtggatgaatgcctgctgcagcccaatgcctgtcaaaatgggggcacctgtgccaaccgcaatggaggctatggctgtgtatgtgtcaacggctggagtggagatgactgcagtgagaacattgatgattgtgccttcgcctcctgtactccaggctccacctgcatcgaccgtgtggcctccttctcttgcatgtgcccagaggggaaggcaggtctcctgtgtcatctggatgatgcatgcatcagcaatccttgccacaagggggcactgtgtgacaccaaccccctaaatgggcaatatatttgcacctgcccacaaggctacaaaggggctgactgcacagaagatgtggatgaatgtgccatggccaatagcaatccttgtgagcatgcaggaaaatgtgtgaacacggatggcgccttccactgtgagtgtctgaagggttatgcaggacctcgttgtgagatggacatcaatgagtgccattcagacccctgccagaatgatgctacctgtctggataagattggaggcttcacatgtctgtgcatgccaggtttcaaaggtgtgcattgtgaattagaaataaatgaatgtcagagcaacccttgtgtgaacaatgggcagtgtgtggataaagtcaatcgtttccagtgcctgtgtcctcctggtttcactgggccagtttgccagattgatattgatgactgttccagtactccgtgtctgaatggggcaaagtgtatcgatcacccgaatggctatgaatgccagtgtgccacaggtttcactggtgtgttgtgtgaggagaacattgacaactgtgaccccgatccttgccaccatggtcagtgtcaggatggtattgattcctacacctgcatctgcaatcccgggtacatgggcgccatctgcagtgaccagattgatgaatgttacagcagcccttgcctgaacgatggtcgctgcattgacctggtcaatggctaccagtgcaactgccagccaggcacgtcaggggttaattgtgaaattaattttgatgactgtgcaagtaacccttgtatccatggaatctgtatggatggcattaatcgctacagttgtgtctgctcaccaggattcacagggcagagatgtaacattgacattgatgagtgtgcctccaatccctgtcgcaagggtgcaacatgtatcaacggtgtgaatggtttccgctgtatatgccccgagggaccccatcaccccagctgctactcacaggtgaacgaatgcctgagcaatccctgcatccatggaaactgtactggaggtctcagtggatataagtgtctctgtgatgcaggctgggttggcatcaactgtgaagtggacaaaaatgaatgcctttcgaatccatgccagaatggaggaacttgtgacaatctggtgaatggatacaggtgtacttgcaagaagggctttaaaggctataactgccaggtgaatattgatgaatgtgcctcaaatccatgcctgaaccaaggaacctgctttgatgacataagtggctacacttgccactgtgtgctgccatacacaggcaagaattgtcagacagtattggctccctgttccccaaacccttgtgagaatgctgctgtttgcaaagagtcaccaaattttgagagttatacttgcttgtgtgctcctggctggcaaggtcagcggtgtaccattgacattgacgagtgtatctccaagccctgcatgaaccatggtctctgccataacacccagggcagctacatgtgtgaatgtccaccaggcttcagtggtatggactgtgaggaggacattgatgactgccttgccaatccttgccagaatggaggttcctgtatggatggagtgaatactttctcctgcctctgccttccgggtttcactggggataagtgccagacagacatgaatgagtgtctgagtgaaccctgtaagaatggagggacctgctctgactacgtcaacagttacacttgcaagtgccaggcaggatttgatggagtccattgtgagaacaacatcaatgagtgcactgagagctcctgtttcaatggtggcacatgtgttgatgggattaactccttctcttgcttgtgccctgtgggtttcactggatccttctgcctccatgagatcaatgaatgcagctctcatccatgcctgaatgagggaacgtgtgttgatggcctgggtacctaccgctgcagctgccccctgggctacactgggaaaaactgtcagaccctggtgaatctctgcagtcggtctccatgtaaaaacaaaggtacttgcgttcagaaaaaagcagagtcccagtgcctatgtccatctggatgggctggtgcctattgtgacgtgcccaatgtctcttgtgacatagcagcctccaggagaggtgtgcttgttgaacacttgtgccagcactcaggtgtctgcatcaatgctggcaacacgcattactgtcagtgccccctgggctatactgggagctactgtgaggagcaactcgatgagtgtgcgtccaacccctgccagcacggggcaacatgcagtgacttcattggtggatacagatgcgagtgtgtcccaggctatcagggtgtcaactgtgagtatgaagtggatgagtgccagaatcagccctgccagaatggaggcacctgtattgaccttgtgaaccatttcaagtgctcttgcccaccaggcactcggggcctactctgtgaagagaacattgatgactgtgcccggggtccccattgccttaatggtggtcagtgcatggataggattggaggctacagttgtcgctgcttgcctggctttgctggggagcgttgtgagggagacatcaacgagtgcctctccaacccctgcagctctgagggcagcctggactgtatacagctcaccaatgactacctgtgtgtttgccgtagtgcctttactggccggcactgtgaaaccttcgtcgatgtgtgtccccagatgccctgcctgaatggagggacttgtgctgtggccagtaacatgcctgatggtttcatttgccgttgtcccccgggattttccggggcaaggtgccagagcagctgtggacaagtgaaatgtaggaagggggagcagtgtgtgcacaccgcctctggaccccgctgcttctgccccagtccccgggactgcgagtcaggctgtgccagtagcccctgccagcacgggggcagctgccaccctcagcgccagcctccttattactcctgccagtgtgccccaccattctcgggtagccgctgtgaactctacacggcaccccccagcacccctcctgccacctgtctgagccagtattgtgccgacaaagctcgggatggcgtctgtgatgaggcctgcaacagccatgcctgccagtgggatgggggtgactgttctctcaccatggagaacccctgggccaactgctcctccccacttccctgctgggattatatcaacaaccagtgtgatgagctgtgcaacacggtcgagtgcctgtttgacaactttgaatgccaggggaacagcaagacatgcaagtatgacaaatactgtgcagaccacttcaaagacaaccactgtgaccaggggtgcaacagtgaggagtgtggttgggatgggctggactgtgctgctgaccaacctgagaacctggcagaaggtaccctggttattgtggtattgatgccacctgaacaactgctccaggatgctcgcagcttcttgcgggcactgggtaccctgctccacaccaacctgcgcattaagcgggactcccagggggaactcatggtgtacccctattatggtgagaagtcagctgctatgaagaaacagaggatgacacgcagatcccttcctggtgaacaagaacaggaggtggctggctctaaagtctttctggaaattgacaaccgccagtgtgttcaagactcagaccactgcttcaagaacacggatgcagcagcagctctcctggcctctcacgccatacaggggaccctgtcataccctcttgtgtctgtcgtcagtgaatccctgactccagaacgcactcagctcctctatctccttgctgttgctgttgtcatcattctgtttattattctgctgggggtaatcatggcaaaacgaaagcgtaagcatggctctctctggctgcctgaaggtttcactcttcgccgagatgcaagcaatcacaagcgtcgtgagccagtgggacaggatgctgtggggctgaaaaatctctcagtgcaagtctcagaagctaacctaattggtactggaacaagtgaacactgggtcgatgatgaagggccccagccaaagaaagtaaaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcgcatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacataactgacttttgtaaatgctgctgaggaacaaatgaaggtcatccgggagagaaatgaagaaatctctggagccagcttctagaggtaggaaagagaagatgttcttattcagataatgcaagagaagcaattcgtcagtttcactgggtatctgcaaggcttattgattattctaatctaataagacaagtttgtggaaatgcaagatgaatacaagccttgggtccatgtttactctcttctatttggagaataagatggatgcttattgaagcccagacattcttgcagcttggactgcattttaagccctgcaggcttctgccatatccatgagaagattctacactagcgtcctgttgggaattatgccctggaattctgcctgaattgacctacgcatctcctcctccttggacattcttttgtcttcatttggtgcttttggttttgcacctctccgtgattgtagccctaccagcatgttatagggcaagacctttgtgcttttgatcattctggcccatgaaagcaactttggtctcctttcccctcctgtcttcccggtatcccttggagtctcacaaggtttactttggtatggttctcagcacaaacctttcaagtatgttgtttctttggaaaatggacatactgtattgtgttctcctgcatatatcattcctggagagagaaggggagaagaatacttttcttcaacaaattttgggggcaggagatcccttcaagaggctgcaccttaatttttcttgtctgtgtgcaggtcttcatataaactttaccaggaagaagggtgtgagtttgttgtttttctgtgtatgggcctggtcagtgtaaagttttatccttgatagtctagttactatgaccctccccacttttttaaaaccagaaaaaggtttggaatgttggaatgaccaagagacaagttaactcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccctttggcaactaacgtagaaactcaacagaacattttcctttcctagagtcaccttttagatgataatggacaactatagacttgctcattgttcagactgattgcccctcacctgaatccactctctgtattcatgctcttggcaatttctttgactttcttttaagggcagaagcattttagttaattgtagataaagaatagttttcttcctcttctccttgggccagttaataattggtccatggctacactgcaacttccgtccagtgctgtgatgcccatgacacctgcaaaataagttctgcctgggcattttgtagatattaacaggtgaattcccgactcttttggtttgaatgacagttctcattccttctatggctgcaagtatgcatcagtgcttcccacttacctgatttgtctgtcggtggccccatatggaaaccctgcgtgtctgttggcataatagtttacaaatggttttttcagtcctatccaaatttattgaaccaacaaaaataattacttctgccctgagataagcagattaagtttgttcattctctgctttattctctccatgtggcaacattctgtcagcctctttcatagtgtgcaaacattttatcattctaaatggtgactctctgcccttggacccatttattattcacagatggggagaacctatctgcatggacctctgtggaccacagcgtacctgcccctttctgccctcctgctccagccccacttctgaaagtatcagctactgatccagccactggatattttatatcctcccttttccttaagcacaatgtcagaccaaattgcttgtttctttttcttggactactttaatttggatcctttgggtttggagaaagggaatgtgaaagctgtcattacagacaacaggtttcagtgatgaggaggacaacactgcctttcaaactttttactgatctcttagattttaagaactcttgaattgtgtggtatctaataaaagggaaggtaagatggataatcactttctcatttgggttctgaattggagactcagtttttatgagacacatcttttatgccatgtatagatcctcccctgctatttttggtttatttttattgttataaatgctttctttctttgactcctcttctgcctgcctttggggataggtttttttgtttgtttatttgcttcctctgttttgttttaagcatcattttcttatgtgaggtggggaagggaaaggtatgagggaaagagagtctgagaattaaaatattttagtataagcaattggctgtgatgctcaaatccattgcatcctcttattgaatttgccaatttgtaatttttgcataataaagaaccaaaggtgtaatgttttgttgagaggtggtttagggattttggccctaaccaatacattgaatgtatgatgactatttgggaggacacatttatgtacccagaggcccccactaataagtggtactatggttacttccttgtgtacatttctcttaaaagtgatattatatctgtttgtatgagaaacccagtaaccaataaaatgaccgcatattcctgactaaacgtagtaaggaaaatgcacactttgtttttacttttccgtttcattctaaaggtagttaagatgaaatttatatgaaagcatttttatcacaaaataaaaaaggtttgccaagctcagtggtgttgtattttttattttccaatactgcatccatggcctggcagtgttacctcatgatgtcataatttgctgagagagcaaattttcttttctttctgaatcccacaaagcctagcaccaaacttctttttttcttcctttaattagatcataaataaatgatcctggggaaaaagcatctgtcaaataggaaacatcacaaaactgagcactcttctgtgcactagccatagctggtgacaaacagatggttgctcagggacaaggtgccttccaatggaaatgcgaagtagttgctatagcaagaattgggaactgggatataagtcataatattaattatgctgttatgtaaatgattggtttgtaacattccttaagtgaaatttgtgtagaacttaatatacaggattataaaataatattttgtgtataaatttgttataagttcacattcatacatttatttataaagtcagtgagatatttgaacatgaaaaaaaaaa

SEQ ID NO: 16 - protein, 2318 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPAAGAHSSPPKARFLRVPSEHPYLTPSPESPEHWASPSPPSLSDWSESTPSPATATGAMTGALPAQPLPLSVPSSLAQAQTQLGPQPEVTPKRQVLA

SEQ ID NO: 17 - DNA, 8089 bp, Homo sapiens
  source 1..8089: mol_type = genomic DNA
gcggcgcggaggctggcccgggacgcgcccggagcccagggaaggagggaggaggggagggtcgcggccggccgccatggggccgggggcccgtggccgccgccgccgccgtcgcccgatgtcgccgccaccgccaccgccacccgtgcgggcgctgcccctgctgctgctgctagcggggccgggggctgcagcccccccttgcctggacggaagcccgtgtgcaaatggaggtcgttgcacccagctgccctcccgggaggctgcctgcctgtgcccgcctggctgggtgggtgagcggtgtcagctggaggacccctgtcactcaggcccctgtgctggccgtggtgtctgccagagttcagtggtggctggcaccgcccgattctcatgccggtgcccccgtggcttccgaggccctgactgctccctgccagatccctgcctcagcagcccttgtgcccacggtgcccgctgctcagtggggcccgatggacgcttcctctgctcctgcccacctggctaccagggccgcagctgccgaagcgacgtggatgagtgccgggtgggtgagccctgccgccatggtggcacctgcctcaacacacctggctccttccgctgccagtgtccagctggctacacagggccactatgtgagaaccccgcggtgccctgtgcaccctcaccatgccgtaacgggggcacctgcaggcagagtggcgacctcacttacgactgtgcctgtcttcctgggtttgagggtcagaattgtgaagtgaacgtggacgactgtccaggacaccgatgtctcaatggggggacatgcgtggatggcgtcaacacctataactgccagtgccctcctgagtggacaggccagttctgcacggaggacgtggatgagtgtcagctgcagcccaacgcctgccacaatgggggtacctgcttcaacacgctgggtggccacagctgcgtgtgtgtcaatggctggacaggcgagagctgcagtcagaatatcgatgactgtgccacagccgtgtgcttccatggggccacctgccatgaccgcgtggcttctttctactgtgcctgccccatgggcaagactggcctcctgtgtcacctggatgacgcctgtgtcagcaacccctgccacgaggatgctatctgtgacacaaatccggtgaacggccgggccatttgcacctgtcctcccggcttcacgggtggggcatgtgaccaggatgtggacgagtgctctatcggcgccaacccctgcgagcacttgggcaggtgcgtgaacacgcagggctccttcctgtgccagtgcggtcgtggctacactggacctcgctgtgagaccgatgtcaacgagtgtctgtcggggccctgccgaaaccaggccacgtgcctcgaccgcataggccagttcacctgtatctgtatggcaggcttcacaggaacctattgcgaggtggacattgacgagtgtcagagtagcccctgtgtcaacggtggggtctgcaaggaccgagtcaatggcttcagctgcacctgcccctcgggcttcagcggctccacgtgtcagctggacgtggacgaatgcgccagcacgccctgcaggaatggcgccaaatgcgtggaccagcccgatggctacgagtgccgctgtgccgagggctttgagggcacgctgtgtgatcgcaacgtggacgactgctcccctgacccatgccaccatggtcgctgcgtggatggcatcgccagcttctcatgtgcctgtgctcctggctacacgggcacacgctgcgagagccaggtggacgaatgccgcagccagccctgccgccatggcggcaaatgcctagacctggtggacaagtacctctgccgctgcccttctgggaccacaggtgtgaactgcgaagtgaacattgacgactgtgccagcaacccctgcacctttggagtctgccgtgatggcatcaaccgctacgactgtgtctgccaacctggcttcacagggcccctttgtaacgtggagatcaatgagtgtgcttccagcccatgcggcgagggaggttcctgtgtggatggggaaaatggcttccgctgcctctgcccgcctggctccttgcccccactctgcctccccccgagccatccctgtgcccatgagccctgcagtcacggcatctgctatgatgcacctggcgggttccgctgtgtgtgtgagcctggctggagtggcccccgctgcagccagagcctggcccgagacgcctgtgagtcccagccgtgcagggccggtgggacatgcagcagcgatggaatgggtttccactgcacctgcccgcctggtgtccagggacgtcagtgtgaactcctctccccctgcaccccgaacccctgtgagcatgggggccgctgcgagtctgcccctggccagctgcctgtctgctcctgcccccagggctggcaaggcccacgatgccagcaggatgtggacgagtgtgctggccccgcaccctgtggccctcatggtatctgcaccaacctggcagggagtttcagctgcacctgccatggagggtacactggcccttcctgcgatcaggacatcaatgactgtgaccccaacccatgcctgaacggtggctcgtgccaagacggcgtgggctccttttcctgctcctgcctccctggtttcgccggcccacgatgcgcccgcgatgtggatgagtgcctgagcaacccctgcggcccgggcacctgtaccgaccacgtggcctccttcacctgcacctgcccgccaggctacggaggcttccactgcgaacaggacctgcccgactgcagccccagctcctgcttcaatggcgggacctgtgtggacggcgtgaactcgttcagctgcctgtgccgtcccggctacacaggagcccactgccaacatgaggcagacccctgcctctcgcggccctgcctacacgggggcgtctgcagcgccgcccaccctggcttccgctgcacctgcctcgagagcttcacgggcccgcagtgccagacgctggtggattggtgcagccgccagccttgtcaaaacgggggtcgctgcgtccagactggggcctattgcctttgtccccctggatggagcggacgcctctgtgacatccgaagcttgccctgcagggaggccgcagcccagatcggggtgcggctggagcagctgtgtcaggcgggtgggcagtgtgtggatgaagacagctcccactactgcgtgtgcccagagggccgtactggtagccactgtgagcaggaggtggacccctgcttggcccagccctgccagcatggggggacctgccgtggctatatggggggctacatgtgtgagtgtcttcctggctacaatggtgataactgtgaggacgacgtggacgagtgtgcctcccagccctgccagcacgggggttcatgcattgacctcgtggcccgctatctctgctcctgtcccccaggaacgctgggggtgctctgcgagattaatgaggatgactgcggcccaggcccaccgctggactcagggccccggtgcctacacaatggcacctgcgtggacctggtgggtggtttccgctgcacctgtcccccaggatacactggtttgcgctgcgaggcagacatcaatgagtgtcgctcaggtgcctgccacgcggcacacacccgggactgcctgcaggacccaggcggaggtttccgttgcctttgtcatgctggcttctcaggtcctcgctgtcagactgtcctgtctccctgcgagtcccagccatgccagcatggaggccagtgccgtcctagcccgggtcctgggggtgggctgaccttcacctgtcactgtgcccagccgttctggggtccgcgttgcgagcgggtggcgcgctcctgccgggagctgcagtgcccggtgggcgtcccatgccagcagacgccccgcgggccgcgctgcgcctgccccccagggttgtcgggaccctcctgccgcagcttcccggggtcgccgccgggggccagcaacgccagctgcgcggccgccccctgtctccacgggggctcctgccgccccgcgccgctcgcgcccttcttccgctgcgcttgcgcgcagggctggaccgggccgcgctgcgaggcgcccgccgcggcacccgaggtctcggaggagccgcggtgcccgcgcgccgcctgccaggccaagcgcggggaccagcgctgcgaccgcgagtgcaacagcccaggctgcggctgggacggcggcgactgctcgctgagcgtgggcgacccctggcggcaatgcgaggcgctgcagtgctggcgcctcttcaacaacagccgctgcgaccccgcctgcagctcgcccgcctgcctctacgacaacttcgactgccacgccggtggccgcgagcgcacttgcaacccggtgtacgagaagtactgcgccgaccactttgccgacggccgctgcgaccagggctgcaacacggaggagtgcggctgggatgggctggattgtgccagcgaggtgccggccctgctggcccgcggcgtgctggtgctcacagtgctgctgccgccagaggagctactgcgttccagcgccgactttctgcagcggctcagcgccatcctgcgcacctcgctgcgcttccgcctggacgcgcacggccaggccatggtcttcccttaccaccggcctagtcctggctccgaaccccgggcccgtcgggagctggcccccgaggtgatcggctcggtagtaatgctggagattgacaaccggctctgcctgcagtcgcctgagaatgatcactgcttccccgatgcccagagcgccgctgactacctgggagcgttgtcagcggtggagcgcctggacttcccgtacccactgcgggacgtgcggggggagccgctggagcctccagaacccagcgtcccgctgctgccactgctagtggcgggcgctgtcttgctgctggtcattctcgtcctgggtgtcatggtggcccggcgcaagcgcgagcacagcaccctctggttccctgagggcttctcactgcacaaggacgtggcctctggtcacaagggccggcgggaacccgtgggccaggacgcgctgggcatgaagaacatggccaagggtgagagcctgatgggggaggtggccacagactggatggacacagagtgcccagaggccaagcggctaaaggtagaggagccaggcatgggggctgaggaggctgtggattgccgtcagtggactcaacaccatctggttgctgctgacatccgcgtggcaccagccatggcactgacaccaccacagggcgacgcagatgctgatggcatggatgtcaatgtgcgtggcccagatggcttcaccccgctaatgctggcttccttctgtgggggggctctggagccaatgccaactgaagaggatgaggcagatgacacatcagctagcatcatctccgacctgatctgccagggggctcagcttggggcacggactgaccgtactggcgagactgctttgcacctggctgcccgttatgcccgtgctgatgcagccaagcggctgctggatgctggggcagacaccaatgcccaggaccactcaggccgcactcccctgcacacagctgtcacagccgatgcccagggtgtcttccagattctcatccgaaaccgctctacagacttggatgcccgcatggcagatggctcaacggcactgatcctggcggcccgcctggcagtagagggcatggtggaagagctcatcgccagccatgctgatgtcaatgctgtggatgagcttgggaaatcagccttacactgggctgcggctgtgaacaacgtggaagccactttggccctgctcaaaaatggagccaataaggacatgcaggatagcaaggaggagacccccctattcctggccgcccgcgagggcagctatgaggctgccaagctgctgttggaccactttgccaaccgtgagatcaccgaccacctggacaggctgccgcgggacgtagcccaggagagactgcaccaggacatcgtgcgcttgctggatcaacccagtgggccccgcagcccccccggtccccacggcctggggcctctgctctgtcctccaggggccttcctccctggcctcaaagcggcacagtcggggtccaagaagagcaggaggccccccgggaaggcggggctggggccgcaggggccccgggggcggggcaagaagctgacgctggcctgcccgggccccctggctgacagctcggtcacgctgtcgcccgtggactcgctggactccccgcggcctttcggtgggccccctgcttcccctggtggcttcccccttgaggggccctatgcagctgccactgccactgcagtgtctctggcacagcttggtggcccaggccgggcgggtctagggcgccagccccctggaggatgtgtactcagcctgggcctgctgaaccctgtggctgtgcccctcgattgggcccggctgcccccacctgcccctccaggcccctcgttcctgctgccactggcgccgggaccccagctgctcaacccagggacccccgtctccccgcaggagcggcccccgccttacctggcagtcccaggacatggcgaggagtacccggcggctggggcacacagcagccccccaaaggcccgcttcctgcgggttcccagtgagcacccttacctgaccccatcccccgaatcccctgagcactgggccagcccctcacctccctccctctcagactggtccgaatccacgcctagcccagccactgccactggggccatggccaccaccactggggcactgcctgcccagccacttcccttgtctgttcccagctcccttgctcaggcccagacccagctggggccccagccggaagttacccccaagaggcaagtgttggcctgagacgctcgtcagttcttagatcttgggggcctaaagagacccccgtcctgcctcctttctttctctgtctcttccttccttttagtctttttcatcctcttctctttccaccaaccctcctgcatccttgccttgcagcgtgaccgagataggtcatcagcccagggcttcagtcttcctttatttataatgggtgggggctaccacccaccctctcagtcttgtgaagagtctgggacctccttcttccccacttctctcttccctcattcctttctctctccttctggcctctcatttccttacactctgacatgaatgaattattattatttttatttttctttttttttttacattttgtatagaaacaaattcatttaaacaaacttattattattattttttacaaaatatatatatggagatgctccctccccctgtgaaccccccagtgcccccgtggggctgagtctgtgggcccattcggccaagctggattctgtgtacctagtacacaggcatgactgggatcccgtgtaccgagtacacgacccaggtatgtaccaagtaggcacccttgggcgcacccactggggccaggggtcgggggagtgttgggagcctcctccccaccccacctccctcacttcactgcattccagatgggacatgttccatagccttgctggggaagggcccactgccaactccctctgccccagccccacccttggccatctccctttgggaactagggggctgctggtgggaaatgggagccagggcagatgtatgcattcctttgtgtccctgtaaatgtgggactacaagaagaggagctgcctgagtggtactttctcttcctggtaatcctctggcccagcctcatggcagaatagaggtatttttaggctatttttgtaatatggcttctggtcaaaatccctgtgtagctgaattcccaagccctgcattgtacagccccccactcccctcaccacctaataaaggaatagttaacactcaaaaaaaaaaaaaaaaaaa

SEQ ID NO: 18 - protein, 2003 aa, Homo sapiens
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 19 - DNA, 6762 bp, Homo sapiens
  source 1..6762: mol_type = genomic DNA
agacgtgaggcttgcagcaggccgaggaggaagaagaggggcagtgggagcagaggaggtggctcctgccccagtgagagctctgagggtccctgcctgaagagggacagggaccggggcttggagaaggggctgtggaatgcagcccccttcactgctgctgctgctgctgctgctgctgctgctatgtgtctcagtggtcagacccagagggctgctgtgtgggagtttcccagaaccctgtgccaatggaggcacctgcctgagcctgtctctgggacaagggacctgccagtgtgcccctggcttcctgggtgagacgtgccagtttcctgacccctgccagaacgcccagctctgccaaaatggaggcagctgccaagccctgcttcccgctcccctagggctccccagctctccctctccattgacacccagcttcttgtgcacttgcctccctggcttcactggtgagagatgccaggccaagcttgaagacccttgtcctccctccttctgttccaaaaggggccgctgccacatccaggcctcgggccgcccacagtgctcctgcatgcctggatggacaggtgagcagtgccagcttcgggacttctgttcagccaacccatgtgttaatggaggggtgtgtctggccacatacccccagatccagtgccactgcccaccgggcttcgagggccatgcctgtgaacgtgatgtcaacgagtgcttccaggacccaggaccctgccccaaaggcacctcctgccataacaccctgggctccttccagtgcctctgccctgtggggcaggagggtccacgttgtgagctgcgggcaggaccctgccctcctaggggctgttcgaatgggggcacctgccagctgatgccagagaaagactccacctttcacctctgcctctgtcccccaggtttcataggcccagactgtgaggtgaatccagacaactgtgtcagccaccagtgtcagaatgggggcacttgccaggatgggctggacacctacacctgcctctgcccagaaacctggacaggctgggactgctccgaagatgtggatgagtgtgagacccagggtccccctcactgcagaaacgggggcacctgccagaactctgctggtagctttcactgcgtgtgtgtgagtggctggggcggcacaagctgtgaggagaacctggatgactgtattgctgccacctgtgccccgggatccacctgcattgaccgggtgggctctttctcctgcctctgcccacctggacgcacaggactcctgtgccacttggaagacatgtgtctgagccagccgtgccatggggatgcccaatgcagcaccaaccccctcacaggctccacactctgcctgtgtcagcctggctattcggggcccacctgccaccaggacctggacgagtgtctgatggcccagcaaggcccaagtccctgtgaacatggcggttcctgcctcaacactcctggctccttcaactgcctctgtccacctggctacacaggctcccgttgtgaggctgatcacaatgagtgcctctcccagccctgccacccaggaagcacctgtctggacctacttgccaccttccactgcctctgcccgccaggcttagaagggcagctctgtgaggtggagaccaacgagtgtgcctcagctccctgcctgaaccacgcggattgccatgacctgctcaacggcttccagtgcatctgcctgcctggattctccggcacccgatgtgaggaggatatcgatgagtgcagaagctctccctgtgccaatggtgggcagtgccaggaccagcctggagccttccactgcaagtgtctcccaggctttgaagggccacgctgtcaaacagaggtggatgagtgcctgagtgacccatgtcccgttggagccagctgccttgatcttccaggagccttcttttgcctctgcccctctggtttcacaggccagctctgtgaggttcccctgtgtgctcccaacctgtgccagcccaagcagatatgtaaggaccagaaagacaaggccaactgcctctgtcctgatggaagccctggctgtgccccacctgaggacaactgcacctgccaccacgggcactgccagagatcctcatgtgtgtgtgacgtgggttggacggggccagagtgtgaggcagagctagggggctgcatctctgcaccctgtgcccatggggggacctgctacccccagccctctggctacaactgcacctgccctacaggctacacaggacccacctgtagtgaggagatgacagcttgtcactcagggccatgtctcaatggcggctcctgcaaccctagccctggaggctactactgcacctgccctccaagccacacagggccccagtgccaaaccagcactgactactgtgtgtctgccccgtgcttcaatgggggtacctgtgtgaacaggcctggcaccttctcctgcctctgtgccatgggcttccagggcccgcgctgtgagggaaagctccgccccagctgtgcagacagcccctgtaggaatagggcaacctgccaggacagccctcagggtccccgctgcctctgccccactggctacaccggaggcagctgccagactctgatggacttatgtgcccagaagccctgcccacgcaattcccactgcctccagactgggccctccttccactgcttgtgcctccagggatggaccgggcctctctgcaaccttccactgtcctcctgccagaaggctgcactgagccaaggcatagacgtctcttccctttgccacaatggaggcctctgtgtcgacagcggcccctcctatttctgccactgcccccctggattccaaggcagcctgtgccaggatcacgtgaacccatgtgagtccaggccttgccagaacggggccacctgcatggcccagcccagtgggtatctctgccagtgtgccccaggctacgatggacagaactgctcaaaggaactcgatgcttgtcagtcccaaccctgtcacaaccatggaacctgtactcccaaacctggaggattccactgtgcctgccctccaggctttgtggggctacgctgtgagggagacgtggacgagtgtctggaccagccctgccaccccacaggcactgcagcctgccactctctggccaatgccttctactgccagtgtctgcctggacacacaggccagtggtgtgaggtggagatagacccctgccacagccaaccctgctttcatggagggacctgtgaggccacagcaggatcacccctgggtttcatctgccactgccccaagggttttgaaggccccacctgcagccacagggccccttcctgcggcttccatcactgccaccacggaggcctgtgtctgccctcccctaagccaggcttcccaccacgctgtgcctgcctcagtggctatgggggtcctgactgcctgaccccaccagctcctaaaggctgtggccctccctccccatgcctatacaatggcagctgctcagagaccacgggcttggggggcccaggctttcgatgctcctgccctcacagctctccagggccccggtgtcagaaacccggagccaaggggtgtgagggcagaagtggagatggggcctgcgatgctggctgcagtggcccgggaggaaactgggatggaggggactgctctctgggagtcccagacccctggaagggctgcccctcccactctcggtgctggcttctcttccgggacgggcagtgccacccacagtgtgactctgaagagtgtctgtttgatggctacgactgtgagacccctccagcctgcactccagcctatgaccagtactgccatgatcacttccacaacgggcactgtgagaaaggctgcaacactgcagagtgtggctgggatggaggtgactgcaggcctgaagatggggacccagagtgggggccctccctggccctgctggtggtactgagccccccagccctagaccagcagctgtttgccctggcccgggtgctgtccctgactctgagggtaggactctgggtaaggaaggatcgtgatggcagggacatggtgtacccctatcctggggcccgggctgaagaaaagctaggaggaactcgggaccccacctatcaggagagagcagcccctcaaacgcagcccctgggcaaggagaccgactccctcagtgctgggtttgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggctgtccaccctcatgcagggaccgcaccccctgccaaccagcttccctggcctgtgctgtgctccccagtggccggggtgattctcctggccctaggggctcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtgggccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattctcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttgccttactccgtccccggagcggggatcacctcaacttgactgtggtcccccagccctccaagaaatgcccataaaccaaggaggagagggtaaaaaatagaagaatacatggtagggaggaattccaaaaatgattacccattaaaaggcaggctggaaggccttcctggttttaagatggatcccccaaaatgaagggttgtgagtttagtttctctcctaaaatgaatgtatgcccaccagagcagacatcttccacgtggagaagctgcagctctggaaagagggtttaagatgctaggatgaggcaggcccagtcctcctccagaaaataagacaggccacaggagggcagagtggagtggaaatacccctaagttggaaccaagaattgcaggcatatgggatgtaagatgttctttcctatatatggtttccaaagggtgcccctatgatccattgtccccactgcccacaaatggctgacaaatatttattgggcacctactatgtgccaggcactgtgtaggtgctgaaaagtggccaagggccacccccgctgatgactccttgcattccctcccctcacaacaaagaactccactgtggggatgaagcgcttcttctagccactgctatcgctatttaagaaccctaaatctgtcacccataataaagctgatttgaagtgttaaaaaaaaaaaaaaaaaa

SEQ ID NO: 20 - protein, 152 aa, Homo sapiens
MKSCKPSGPPAGARVAPPCAGGTECAGTCAGAGRLESAARRRLAANARERRRMQGLNTAFDRLRRVVPQWGQDKKLSKYETLQMALSYIMALTRILAEAERFGSERDWVGLHCEHFGRDHYLPFPGAKLPGESELYSQRLFGFQPEPFQMAT

SEQ ID NO: 21 - DNA, 1534 bp, Homo sapiens
  source 1..1534: mol_type = genomic DNA
ctgcactctccgacagctactgcgctaaaagcgctccttccctgagcttcgggaaagagttcatcttcctgcaaaggagtctcaggctttcccagaggacttgaaaggccttcctcgaaccagccacaccaaactctgctgcagaaggtttccttctctttttcaacttcatgttgagaaaatgactttctcttgagcatctcattttcccctaaatttgggcaagtgaagagatatcagcctggtcatccagtagaacagaaggccgagtcccgcactcccccactgtaaactatttgattgcacgtgagttgctttgtttatgacttatttgctcagaagaggcacgttgggaagcggctcgagagaccagcccacgcgcaggtcctgagcgggcgggcgtgcgaggtcggcgcctcgctgcttggggccggggatgaagtcctgcaagcccagcggcccgccggcgggagcgcgcgttgcacccccgtgcgcgggcggcaccgagtgcgcgggcacgtgcgccggggccgggcggctggagagcgcggcgcgcaggcgcctggcggccaacgcgcgcgagcgccgccgcatgcaggggctcaacactgccttcgaccgcttacgcagggtggttccccagtggggccaggataaaaagctgtccaagtacgagaccctgcagatggccctgagctacatcatggctctgacccggatcctggccgaggccgagcgattcggctcggagcgggactgggtgggtctccactgtgagcacttcggccgcgaccactacctcccgttcccgggcgcgaagctgccgggcgagagcgagctgtacagccagagactcttcggcttccagcccgagcccttccagatggccacctagggcgcgcgcctccgcgggggtgggtgtccggcagccgctccgagcctcggccctgccccaagtagcccagaagcctccggcggcccaggattctaaggatgcaatcctcgaggaaaattagtcgattctcagattacctttattcgcatcatcagacctatggacgcaatcatttaattgcctttcttttcccctcctcctttgtattttgtagatttcattaatggatcttgtgaatgggttgattgctgtgaaaataatgccccctttccccttttctgggctactttgagggaaaacaatcttaagaaaaataggattaagctattctgttccagtcctcagagaaataatcactttcttaaactttgtgagtttgtcctgttcgggtgaagttacagtatccattacttgtgtttgctcacaacagagctaccttcctgttgtgtaaatgcgtttttgctttagtgcattgtgtgtgcaagcatgaagtagaaacactttttttttctgggtacagtacatgggtatcggtgctctgtatttttttaaactgtgtacacattattaaaatatacattttataaaatataaataaaaacgtggatttgtttttcatgccaaaaaaaaaaaaaaaaa

SEQ ID NO: 22 - DNA, 1385 bp, unidentified
  misc_feature 1..1385: note = Description of Unknown: Atoh1 enhancer polynucleotide
  source 1..1385: mol_type = genomic DNA
tccaaggtccggcaatgaagtttgcataacaaacgtttggcagctccctctctcacaccccattaacaagctgtaacatatagctgcaggttgctataatctcattaatattttggaaacttgaatattgagtatttctgagcgctcattccccatatgccagaccactcctgccatgctgactggttcctttctctccattattagcaattagcttctaccttccaaagtcagatccaagtatctaagatactaccaaaggcatcaactatgtatgcaagttaggcatgcttaatatcacccaaacaaacaaagagtcagcacttcttaaagtaatgaagatagataaatcgggttagttctttgggacaccgctgttgttttccagagtttttctatactttaagcagcttgttttatattctgtctttgccctcagccagctaacattttatttgttgagggttttggctcaccacacttttggaaacttatttgatttcacggggagctgaaggaagattgtttttggcaacaggcaagtttaacacgttcttcatggggcattgcgaatggcacatctaccagaaagggagggggagtaacttcctcgtgctgaaccagcaggagaccagagctttcctgaggtcttcctattgattttaaagatttaaaactgagccccaaagttgtaatgttattgaagtttgtcttggaatatacatctcctctgctaacttaaaagttcaagaaaggaaaggaaagaaatagaaccccttgctaactacaacctagactgagaggtgaagatcgcgggcaaagacaggtggtcactgaaacgtttgcagttcttttcttccgaaggcttaggacacagggtaaggaggagctaaaataaagccgagtgtacgtttagtcttctctgcaccccaggcctagtgtctccccaggcaaggagtcaccccctttgcttctggctcctaactgaaaaaggcaaaagggagtggagaatgggttaaatcccaggacacaggggagaggcaggggaggagagaagtcggaggaagataaaggaaaggacaggaaccaagaagcgtgggggtagtttgccgtaatgtgagtgtttcttaattagagagcggctgacaatagaggggctggcagaggctcctggccccggtgcggagcgtctggagcggagcacgcgctgtcagctggtgagcgcactcgctttcaggccgctccccggggagctgagcggccacatttaacaccgtcgtcaccctccccggcctcctcaacatcggcctcctcctcgtagacagccttgctcggccccccaccggcagagtttacagaagccagagcctctcgccgttcccccg

SEQ ID NO: 23 - DNA, 8501 bp, unidentified
  misc_feature 1..8501: note = Description of Unknown: Pou4f3 promoter polynucleotide
  source 1..8501: mol_type = genomic DNA
agggggtggcacggcgcggcgcggcgcggcgcggcgcggcgcggcgggtgtctcacagttggcctctggctccctggtccctctgggcatctcggatcctccctgggctgggcagacaatgagaggcagcggccgacaggcgagtccagtagcagctgtgcaggcggaggttacatgtgagaagtttgtgaaagaaactgagagaaagagagaggaaggcaaggcaggctgcagacacctggccccggtgcctcaccagctgccctcctgcgatggcatcttgagttcgggcagctcctatcgaagccatcttgcctggcatcctccccgccccctctccagcatgcctaggtgaggctgagctgatggaggcttagctgtggttccaactaactgccctgtaccagcagaggaagctagcttagttcaaaacctagtaaagcaaacactctgtcaggggaacttgccttccccttcacattgagcattcctctggcaggagtcctgaagaagatttattaatatgtccattctaaaatgacatagacttcttaaggactgtttgggacacataagaactgataagactcaccctctaaaagtctccagagtctttgaaggaacagacattcatcgtcacatgcttttgaagagataacagagcagggctggagaaatggctcagcagttaagaagacttccagaggacctggtgagattcccagtcccctgtaattccagttccagtgaatctgatgccttcttctggccagcagcactcacgctagacacaaatgtggtatacaaacatacatgcaagcaaaacaaaagtgtgtgttaagtgtttgtgcatcatgtgcatactacgtgcctggtaaggccagaagaagatgttggatcccctggaactcttagagatggttgtgagctgccatgtaggtgctggaaattgtatcagggccctctggagaggtcaataaatgctcttaagtgttgagccatctctccactcctcatatacattaaaggaataatttttttgtacaggctacaggggaggttgaggcaggaaggtctcaaggccaaggccaggcttagttacatagagggaactgtctcaaaataagagtggaggatctggagaaatggaaagagcactgttgttcttacaggggaccaattcctagcatccacatggaggtccaagatccttttctgactctgaaggtatcagacactcatatggtagacatacatacatgcaggcaaaacacttatacacataagataatctgaaaaaaattaataaaggggggaatctaactcattggcagaccactcgccttgaatgcacgaggccctaggttcagtttatcatatgcagagagtagagttagtcacattgggatagacaataagcagagagaagcgggatgctagctgtctcctttcttccattgtgtgtgtgtatatatatatatgtatacatatatatacacacacacatatacatacacacacacacacacacacacacacacacactgtatatactgtagttgtcttcagccataccagaagagggcatcagatcctattacagctgattttgagccaccttgtggttgctgggagttgaacttaagaccttggcaagagcagccaatgctcttaaccactgggccgtctctccaatcccctccctttctccattttgttcctttgtgtatacccgcccttgggatgatgacacttggatccaaggtggatcttctctcctcgatcaatccttcaagaaacacctcacacgtgtccttcttaagttttctcttttttccaagacaggatttctctgtgtagccatggctggcctgtaaccatctttatagaccaggctggcctcgaactcacagagatccacctgctaatgaaagaactcagtggggaaacccccactcagctcccgattcggcgtgcacccaagaatcgcgaatagaacacaacaccttgatgtaacaacaagaggttttttaatggcggagctccgggtcgaaacgtatctcacacaacaggagacagtggattcgaccacgaggcttagaagctaggggtttttatagaaaaggagtggggctggggaaggaattgacgcggtttcacatgattggtccatttaaacatcagcagcctgtaacatttaacttaggtcagaggggtgggagctagggaggcgaagggcttgcccgggcatgtcctggtctgttctgctgtgttctcagccccaggtttcaaagcgcacaaacaactctttgggctatttaacatacattacatgaattacagttttatttcctttcacttctactgtctgagtgcttggcctaaaggcgtgctccaccacctggatccttaggcttggcaaccacatctactccttgttacttaacactgaaatacaccactttagccacaacattccatcctgtcccccaagtgttccgatatatcgtatctcataacgcaaaacattctatccaatttcaagaattcccatagttataaaagtcccaacatgattgaaaagtccaagttcaataaaatctctgggtctccaagaaaatccttaaatgtgagttctggtaacaatccagaaatattctacatccaatatacaatggtaccaggtgaacatcacattccaaaagacgggaaggagagcatagaacaaatggatgggaccaagtcaagactaaaacccaggagaggaaatacgaaactctgcagctccatttgtagcaccgagggcacgtcatataatgacgtgcggttgaagatggttgttagctgctgtgtgggtcctgggttctctggatgaacattgactagtaaaaaaatttttttgaatgaattaatttacttatcttctgggtgaggcaggatctgaatgtggaggccagagggcaactgtgggagtcacttgtctcctatgtgttagatctggggagacaactgaggaatccaggtccccaagcttcatgccaggcacctttactagcggagtcatctcactgaccttggcactcatcactttaataacagttaccttttaattcattcatttaatttaattaatgataattccacagcattcaactcaatcctttttctggccatatggttttcaaaatcttttgctgtcttgagtctcattataaacccaactaaacacaatgaccacaacacctaactaaatgctatgctgtcttgaaatgtcctccaccaaatgtgtctgtcacccatgtttccctacacacacaaagtttatggacacagacaaaatactttgccagaatagaattcaaatgatctttagtcaagctcccaatagagacctcatttgcatcgaaaacctcagggggccaagctttcatcctttgagtctagtccactgaactcttgtgcatctagaccactgaactccagacagaattccccaccaagctctgctcaccacactctcagcacctctagtctaccttcccctaccaacccccttcaagcaaacaagttccaaaggcgtgagaactagctggttagcaagaatgtaacttctgggtgccaactttctgtattagatagttttttcctcactaatcagatacttgatagaaacaacttaagagagagaagatatattttgtatccagtgtcagagttttcagtgtgagagagctagagcacagaattctgtgctgggtcccgaggtagagaaaggagaatgctagctcctgtcaactttccctctttaacccatttattccagcaaagtccaaagccatgaggatggcaccactcacattcaagatgagtctttttttttttttttgccccttacttaatcctcacactcacaggtagatctcaccagtctttcaggtaattctaaatatagtctagttgacaaggaggattaaaccaccacagcccttgtcaaaacctctgagtccgagttttcgtgctctgttttatgaaactggagttgcttctttcttattcattctaatgttgaaagccactagcccacagaaaaatgtccttgtccctgatttgaatctctctggagaacacagtgctctgagtcctgatttatgtggcaatctcatgtcctggccaagatcttggttttttgttttttgttttgttttgttttttttgttttttttttttttttttttttttttgagatagggtctcacttatgtagccctggctgtcctgaaactccctatgtagaccaggctggccttgaactcacagagatccacctgcctctgcctcctcaagtgccactacactgccttaaaccctaagatcttgaaacagtgctctctgtatctgcagaacctgccatgaaatccagctagcagtgggtatttcaaagaccttatagaatgaaaaaaatgtgactcctttggtgcccttgactgaaccctgagattatttttggactcaatgggtgtagggtatttccttatttgtttgtgctctaggtggggtccccattatgtagcttagctgaccttgaactcttgatgtagaacagactgccccccccacctgtctctctatgtgtgttttaattacaataaaatgccacggtataagcgtaagtttacctcaggccaacctctcttgcatgaccaactacaaatagtagttgactggtttttattagattttcatctaattgaatttaagtttgtaagttctgaaaaaaatgtgtcctaaggcttatcttgtactttcatccccccccccgcccaccaatctattattttatgtgtatggaggttttgtatgtatatatgtttgtgtacctcctatgtgaagtacccctggaggtccgaaaagggcatcagattcattggaattaacaaatagttctgagctaccatgtgtgtgctgggaactgaacttgggtcttctgaaagacccagccaatgctcttaatctcagggtcacttctacagaccctgccgttgcaatttttaaaaattatttgttgttcccttgtcatcatgtgtgtgtctccctgcccttgttccacccccattccccaccgatttccccagcattatctaaagaccttgataaggcctgtggtgtgacagaaaacagttgataaaggagatcagcattacctttttacaggtcctttaaaaagccaagtaagtacatgtatgactacacttaattgatttttaaaatttcagcccctttctgtagctgggtgtctttgcatgtagtttcactctgtctccatcaataggtttatgttcaggcaaagccataatcagattttctaagagtcatctccagtgacaattattcaggtcttattactattaattattatttaatatttaattaattgattaacaatcattattaatcattagtttttcatgttattattcgtttcgttttgtttaagatctcgcacagcccaagctggccttgaactcagtctgtcatccatgtgagtacagaactcccagtttccttcacccacatcccaagtgctgaggttacaggtgtgaacagccacatctatcttacagatttacaatggtttcatgcccttttctgatgctatagaataaagaaaatgtatttttgccagctaaagaagaaaaaaatgattcccttacaatttgtatttcttatctttcaggctaattgatgattgagttccaaccaggttgactatagcaatgtataaaagattaattttaaagtgtgacacaaaaataaatattcaaaatggtggacacacacacacacacacacacagaggctagttttactgataggtgtcagcttactactgaccattgctagtggaccttatgggaagaaaagacagaaaggtgttttctaggggcctgagggaagtagtggtattgtcctggtcagcattttttgatgacttattttattttatgtgcattgatgttttgcctacatatatgtctgttgaaccgcattggatcccctgaaactgcagttacaggctaccatgtgggtgttgggaattgagcccaggtcctctggaagatcagtcagtgttgttaaccactgagccatctctccagtcccacctagtcagtattttccaagtccatgagtgacagagcagtgaaggggatgggtatgaatgcgtctttcccaagtattcctcttcacagacaacgaggatattatggcatgaaaagagacccttaagaggctccatggggtcaggcaaaatgtaggtccactacctccatgtttattcccgcttcccttttggactccacactggtcaggaaacaatactgaggagtcttaaaagctagtgggagataaaaatcacccatgtttgacttctggggacttgaggggcttgtgttgtggcacaaagccagtaataaggagaggaacagttcacatctgcagagagaccacatccctggagagactgcaggctgttctttaggttaaaaaggaggtgctaacatttgttccatcctttcaacatgttggatgccgtttcaagtgcttaccaccaagtaaccctcactacagtaggggaaactactgtatgcatctgtatacagatgggcactgcagtatagaggcctcaagatgactgtctcctggcctctcagccagtagaagtagaagcagcagtgtgctctgagtctgatggcagcatgcactgggttgtgaagaagtgtcctgagtgcacagtgaggtcatcatcatcatccttctcctccagcaagacccaagaaagctacctgagtaccatttcagttggcctccctcacagattgatgattgattgattgattgattgttgttgttgttttgccacagcaaggaaggacctagaatgtgttttgggatgagttgagaaaggactggctggcagtcctgaggaaagagggggagccaggtcagtgtagggtgtcatcttagactaaattcatccctgggtcctagactttccacactctgccccctcagtgttaagattctgggaaacccagcactcttgagcacagagacagtgctgcaggacacagctgggcaggaagaatttctcttccaacccgaaggcagctctccagccggccacagtcccaggattccccatccattattcatggtgtttattagctcccaggaagggagaggaaagaaggagaggaggcgctagggaaataggggacacagaggatggggcagagacaaacttaggacccttaagttgggggaacttctaaaggaagaagaggaggctaagaggtgaagagcccaaagtcagacatggtgttggctccaaaccagtgagtgaatgacactgctcacaggtaggagatcttgggctaacttatttggctcttgtttcctttggctgtctaatgggatagcagtagctactctgtagtgtcctgtaggaacgttggtcctatacactaacaatactttttaaatattgagtgctcaatacttttctcattatgttattattattattattattattattattattattattattatcattgcctcgcacaaatcagtagctcaaaaatgatagctgttgttacaggtattggggctgctaagctctacagaagtatgcagaagaattgctccagctaggccagctgtcctctactgttccttagcaagctttctggggggtgggggggaaagggagaacttggcccacaagttctgtttctcagatgaaagctgaaacagccagggttaggaagaagccacaggcttgtggatcaggaagcctttcttccacccaagctcacagaggggagtatcacccgataagcattctggtaacctctggtccttgcccaggctggtttctgagtttggaggcaccttttcgctatgcactgggatgcaggctttgagcttcagactcagagcaccttgaaaatagccagtttggggtgtgtgtgtgtgtgtgtgtgtgtgtgtgtgtgtgtgtgtgtgtctcaggagcttaagtctgccctttcatccaggcagctcgagctgggacaggacagaggtttaggactttatgcaaagaagtccaggaggaagaagaaagaatctgtaaagtctggcaagctggagccaggtggggcggggcggacaggaagaggccctgccaggccggggtataaatgctgtggaggggggcggccgcatcaggctcagagtggcgcgccgagacctgcggtcccgccttgcctcccgggccgcccctgcgagtcccgggcgcgtgtgcacgtctgcgcgtgcccgggcccttcctggcagactgcttgtaagatgagtgaagaagcaggtgggggagaggggaggcagcaagcgagagggcgaggggagcgctggcgctgagcggcgctcacttggagcgcggagagctagcaagacga

SEQ ID NO: 24 - DNA, 3833 bp, unidentified
  misc_feature 1..3833: note = Description of Unknown: Myo7a promoter polynucleotide
  source 1..3833: mol_type = genomic DNA
ctagagggatctgtctgtttcatttttcccgtaccccgcacccccccacacacaagggtaaagacagagggcacaatgggtagctgacctctggtcagaaggatgagcaaggaaggccttgggaatcacagataaaagctggccttgctggttacctagtgagtcagagcccagctctgtggaatccttcaggttccctgcttccagtcagtgtggggctggctttgctgagctctgcccatctcaggccctggggacatgggggcacacagttcctgctctgcagcagccttccagcaactggggaaaatgtacaaatcatgtctgatcgaagtactgtagtgtccttgataagcagtgtccctaggagctgactcttagtctctgagttcaagatctgtgccctgttctagtggagaggagagacttacatgcaggccacagacggaggtggcagagcagagatcctgggaaggtgcggtagctagagctaggaatttatgggcagggccacgggtagaagctgggtcgggttgtggaaaacgaaatgatggaatacgacgatagattggatttgaaggggcgagggttgtatgaactaagaccagttctgcagtggttcagatgtggtgcccttaagaggctcaggtgccagaaaccagtgccccttctggatagggcagaaaacaggcagaacctggagaggtacggtggagcatgaggtggtaaggacacacaccatctcaggtggtttgttttggggaaaacaagcatggatttctaggtttctccgatcgggtgacctgcttagccgagtttgggtgctgaggaaatgtctccgttatggttctgagaccactttctcacccacccactgccttcccagcatgcacctcagcctcaacgaagcacacctgccccagttgccgccttgtcgattgggctctggatgctccaacctgtgtctctcccactctgtgtaggctcatgctactcacctgactcataagtatcagtttcgtaggtgaaggctgcgttgggtgcaagtcaaccttggaccctggtctcactgtggcaccctggtatgaggaagttgaccggcttttccttagtcttgtagcagttggctgtgcccagcaggtggcaccattgcaccattgattctccccactccataccactatagattccaccccaccctattcccctccccaccttaacccacccacccccaccgaaaagcagctttcctgagtagatgtcccagctggcaagtgtgggcagaagaaggggccaggtctcaggaggaggaggaggaggaggaggaggaggaagaggaagaggagtccttcagcctccttcctcatctaccatgatgagtatttgtgtcctgttcattcccacccactcccttttttaaatcacacatttaaatcacacacacacacacacacacacacacacacacacacacacacacacaccgggtaagtctgtcctgcgtgaggtggctcctactcaggtggctttgcaaactgtctggataacagcacactcaagactcctaggacaggctgtgggggccagttgtagagcctgggggtggggtgcatcttggggagtcctggtttggatgttgtgtccagccaaggctccaggtattgccaagcctgctcatttatatggtctctagtagtgccctgacgagggaagctgggtgagcaggggaggctactgggaactgagacccagcaaaatcatgaggaagatgggacgtgatcaggtgtcctaaccatgcagagatggcaggtagtaacacatgtgacaagagaccctgaggtcctgatggttggccccaggcccgaggttcccactggccagcagtgccccctggagcttctatgccttgcatccctgctggttagctttacacagcaccttgggcaacctctagacgttagtcagcagccccagcacagcccgcccctcatgctgatgtcaccacatccagaccttcgaggcccccagggctccgcctcctgggagaaggctttggagggagagggcgggtggcagtgcaggctggacagctgccctgaacagaaagaaagagtgacccagggagacaagaaacagagtagcccaagggaagcccacagcagcagcagatcaaggctcaagctggagctgaaaatttgcaggctccagcctcagcttccagagtcctcctgacctgtgacccctggctcctggctgggaggtggtgactcggagggtgtggataaaacccaggtaaggatgggctgccctggctggaacttttggactggggaggagttacaggctggggcctcctctggagcctctgcccttaggttcctgggactggactcccctgaacctacaggggccttgccaccataggcatggaaagaaaatgccccgagccgtgcacatggaggcttatgccctagatgagggaatagctgaagagcacagggcctgtggagttgtttccgggttggagctgggtcaggggtatccagaggcaggggtgaaagatgggagtggaggctgggggggcaagtgtggatgcctctctctgcttggtcttttggtcatctccctgataatctatctctccttgcacacgttgtttcttctttgtgtgccttacattctctatccgtgacccaagaacatgaaggttaccatctggggttgcccaggggtcaaggtaaaatcagttgtgggtctagggaaaaaaacaaaacaaaacaaaaaactatggttctttgttctcaatttgaaaagcaacagtcttacacactagtctagtctgggatggagaggtagagcctcatgcatggggtagaagagacctgagcttttccatgggtcttcagctctaccctccaagtctacagtcccagtctgtcctgcagccttgtaccagcatgagtgaggcaggggccaggcagcctcttcagacgccagctctcataactactgctgctgcctttgggattatccccctgccaggaccccagcgtggtgtaggtctcccgaaggaccatatgcccatagagacccagggcgtggggtcagggctcaggtgtcaggcagtgtcgtgggcagaggatcagatgtcccagtgcctctgaatggacccttgtgaaaacttaatgctcactcgaggggcaggccaggcagggatggaggggcctcacaccaagacctatgggctggggctgcttctggtgaccacactcgaggcagcaaatccatctcgagcctattcctgggtccagctctgtgctggatgaggtaagagatgagggccacgaacaggtgttagatcattctgccatgtgtcctaaaactcctaaagcagtcctgaaccaggagcagagtccaaatcttacttagtgccaagtcttccctgccctgctgtgtgaccttgggtaagagatagtccttctctgaactttgggcagcctcaggacattagtccctgtcccagcacagcccacccctcacgctgatgtctggtacagggctgactacactcctttcaaagccctttgggatgacgagggcagctttgggcagtggaggtggtgacggtaactgaggctctggtcacatagactgagctttaatcagaaggatggaaaccaagaaagatttccgtgagggaggaggggtaggggtggggttggggaggagtctaggtggccctgcagatccaagctgcctgggctcagggcgtgccatggtctcttcccacagagctgtgtctggtcactccggcaggtgtgctgacgtagaag

SEQ ID NO: 25 - DNA, 1600 bp, unidentified
  misc_feature 1..1600: note = Description of Unknown: Hes5 promoter polynucleotide
  source 1..1600: mol_type = genomic DNA
acctctgtcagagctcgggtgagggttgtggcagggactaagggaggtgaattctgcacttatatcttgcactggacagaccatcctgtgctgcctcaaagccaagccttcccctctgtcccagatgcttccatccatggtaacccaacaaccccttgtgctaagagcttctggagcaagaccttcggaatagatcatttacagcagtgggtcactaccatagcaggagggcgtttccctagtgggcactgtgaaggcccttcagttcctgagctcagacatggctgacatccatccagagaacctggtttcattccacactggctattgtgctctgatgccagcctcctggtttgggcctggctaggtcttaggttgaccctaggaggaagcacggggccttatctcctccctcagcccctgcagagtattggggaaatagtacaggttccagatggagggacgctcctacatggccatcccctaggtgtctagggtgtgactagtaaggtcacctggttaggaaccacggttatcagttcttacaaagaggcccatccacactggcacagcattcatatgggggcttatttttaggtctcccaccttctacctgccaacacaaaatgctgaatgaaactggaacacacacacagaggcatgcacgcgcgcgcccgcacgagctgagggggatgtcatgagaggagttaagggcagccatggtttcctaaaagccacgcgggagtagtgatcacggacagcatgaaggaggcacggtcctcctgtttcagagatgcatgtggcacctggtgacacaagagcagtccagataccctgtgggttcaggggttcctgagggcttcctaggtatggggctttgtgctaggcattatccaaagcaaattcagctagcctagaagtacgcttggcaggctccgcgcgtggccatttctagctgcggagaagtcactcctctcaactgacagaactggcaataaagcgcctaaattctaaatacatgagtccctctctcatcctgtacctgcgaatggcatggcggcagcatctgtttgagagaatgtggcccactttaacccactgtgcctgtgggcaacaggtcccagctggggagggtgcccactcctctggaagtcgctgtctgttcattgtaaaactctcatttctctgcgcctgtggcctctgccagccagcggtggggagcctctggggagtgggagggaagaagggagagaagggggggggagagcactccttcctgccctccccacctccccgcggcctgggaaaaggcagcatattgaggcgcggggctctcagcatcaggccccgggatgctaatgagggcgagcgcgttcccacagcccggacattgtgccgcgcggcccacctgctcctcggggagcgaccattgtgcccgcgccaattcacaggcaatttagcgtgcgctaatgggccggcgcctttgtgcggccggcgccgccattggccgccgagtgtgggaacggccgcggcgcccggaccccaggcgccgggccgctgcccgcgcctatatagggctggcgtgctggggtccaggtcg

SEQ ID NO: 26 - DNA, 2211 bp, unidentified
  misc_feature 1..2211: note = Description of Unknown: GFAP promoter polynucleotide
  source 1..2211: mol_type = genomic DNA
aggagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggatggagaggagacgcatcacctccgctgctcgc

SEQ ID NO: 27 - protein, 11 aa, synthetic construct
KEAPPAPPQSP

SEQ ID NO: 28 - protein, 12 aa, synthetic construct
GKEAPPAPPQSP

(19) United States
(12) Patent Application Publication
(10) Pub. No.: US 2025/0099539 A1
(43) Pub. Date: Mar. 27, 2025

(54) METHODS AND COMPOSITIONS FOR REGENERATING HAIR CELLS AND/OR SUPPORTING CELLS

(71) Applicant: Massachusetts Eye and Ear Infirmary, Boston, MA (US)

(72) Inventor: Zheng-Yi Chen, Chestnut Hill, MA (US)

(21) Appl. No.: 18/905,706

(22) Filed: Oct. 3, 2024

Related U.S. Application Data
(63) Continuation of application No. 16/842,309, filed on Apr. 7, 2020, now Pat. No. 12,156,899.
Application No. 16/842,309 is a continuation of application No. 14/426,520, filed on Mar. 6, 2015, now abandoned.
(60) Provisional application No. 61/698,246, filed on Sep. 7, 2012.

(51) Int. Cl. A61K 38/17; A61K 31/65; A61K 35/30; A61K 38/16; A61K 48/00; C12N 5/0793

(52) U.S. Cl. CPC A61K 38/177; A61K 31/65; A61K 38/16; A61K 38/1709; A61K 48/00; A61K 48/005; C12N 5/062; A61K 35/30; C12N 2501/42; C12N 2501/606; C12N 2710/10341

(57) ABSTRACT

Provided are methods and compositions for inducing cells of the inner ear (for example, cochlear and utricular hair cells) to reenter to cell cycle and to proliferate. More particularly, the invention relates to the use of agents that increase c-myc activity and/or Notch activity for inducing cell cycle reentry and proliferation of cochlear or utricular hair cells and/or cochlear or utricular supporting cells. The methods and compositions can be used to promote the proliferation of hair cells and/or supporting cells to treat a subject at risk of, or affected with, hearing loss or a subject at risk of, or affected with vestibular dysfunction.

### CROSS REFERENCE TO RELATED APPLICATION

This application is a continuation of U.S. patent application Ser. No. 16/842,309, filed Apr. 7, 2020, which is a continuation of U.S. patent application Ser. No. 14/426,520, filed Mar. 6, 2015, now abandoned, which is the U.S. national stage of International Application Number PCT/US2013/058626, filed Sep. 6, 2013, which claims priority to and the benefit of U.S. Provisional Patent Application No. 61/698,246, which was filed on Sep. 7, 2012, the entire

### GOVERNMENT FUNDING

This invention was made with government support under Grant Number DC006908 awarded by the National Institute of Health. The Government has certain rights in the invention.

### SEQUENCE LISTING

This application contains a Sequence Listing that has been submitted electronically as an XML file named “00633-0313003_SL_ST26.XML.” The XML file, created on Oct. 3, 2024, is 160,800 bytes in size. The material in the XML file is hereby incorporated by reference in its entirety.

### FIELD OF THE INVENTION

The field of the invention relates generally to methods and compositions for inducing inner ear cells to reenter the cell cycle and to proliferate. More particularly, the invention relates to increasing c-myc and/or Notch activity within cells to induce cell cycle reentry and proliferation of hair cells and/or supporting cells of the inner car.

### BACKGROUND OF THE INVENTION

One of the most common types of hearing loss is sensorineural deafness that is caused by the loss of hair cells or hair cell function. Hair cells are sensory cells in the cochlea responsible for transduction of sound into an electrical signal. The human inner ear contains only about 15,000 hair cells per cochlea at birth, and, although these cells can be lost as a result of various genetic or environmental factors (e.g., noise exposure, ototoxic drug toxicity, viral infection, aging, and genetic defects), the lost or damaged cells cannot be replaced. Hair cells also are found in the utricle of the vestibule, an organ which regulates balance. Therefore, hair cell regeneration is an important approach to restoring hearing and vestibular function.

Studies of regeneration of hair cells in mature mammalian inner ear to date have focused on transdifferentiation of existing supporting cells. Supporting cells underlie, at least partially surround, and physically support sensory hair cells within the inner ear. Examples of supporting cells include inner rod (pillar cells), outer rod (pillar cells), inner phalangeal cells, outer phalangeal cells (of Deiters), cells of Held, cells of Hensen, cells of Claudius, cells of Boettcher, interdental cells and auditory teeth (of Huschke). Transdifferentiation of supporting cells to hair cells by overexpression or activation of Protein Atonal Homolog 1 (Atoh1) in supporting cells or by exposure of supporting cells to Atohl agonists is one such approach to generating new hair cells. One limitation to this approach, however, is that transdifferentiation of supporting cells to hair cells diminishes the existing population of supporting cells, which can impair inner ear function. In addition, overexpression of Atoh1 in aged inner ear or flat epithelium, which lacks supporting cells, is not sufficient to induce hair cells. Furthermore, it is not clear if all types of supporting cells can be transdifferentiated into hair cells upon Atoh1 overexpression.

Other studies of hair cell regeneration have examined cell cycle reentry for hair cells in embryonic or neonatal mice by, for example, blocking Rb1 and p27kip1. However similar manipulations in the adult inner ear have not induced cell cycle reentry. In addition, the hair cells in embryonic and neonatal mice that reenter the cell cycle in general subsequently die.

Over 150 types of genetic deafness are due to mutations in genes that affect both hair cells and supporting cells. For example, mutations in Myosin VIIa (Myo7a) cause hair cell stereocilia abnormalities that lead to permanent deafness. Mutations in GJB2 (connexin 26) cause damage to supporting cells that lead to the most common form of genetic deafness. Approaches (e.g., gene therapy and anti-sense oligonucleotide therapy) have been developed as potential treatments for hereditary deafness. However most of these defects occur during embryonic development. By birth, affected hair cells and supporting cells already have died or are severely degenerated, making intervention difficult. Therefore, to treat genetic deafness, there is an ongoing need to regenerate hair cells and/or supporting cells in utero and after birth, which can be combined with other approaches to correct the genetic defects underlying the disease.

In addition, inner ear non-sensory cells (e.g., fibrocytes in the ligament) play essential roles in hearing. Inner ear non-sensory cells can be damaged by factors such as noise and aging, which contribute to hearing loss. These cell types, like many of those in the inner ear, lack the capacity to regenerate spontaneously after damage.

Because spontaneous regeneration does not occur in the mammalian inner ear, recovery from hearing loss requires intervention to replace any inner ear cell types that are lost or degenerated. Therefore, there is an ongoing need to regenerate hair and/or supporting cells within the mammalian car, in particular in the inner ear, to replace those lost, for example, by genetic or environmental factors. The regenerated hair and supporting cells may be used to slow the loss of hearing and/or vestibular function and/or partially or fully to restore loss of hearing and/or vestibular function.

### SUMMARY OF THE INVENTION

The invention is based, in part, upon the discovery that increasing c-myc activity, Notch activity, or both c-myc and Notch activity in an ear cell, for example, a cell of an inner car, promotes cell cycle reentry and proliferation of the cell. When the cell is, for example, a hair cell or a supporting cell, it is contemplated that proliferation and subsequent differentiation of the cell into hair and/or supporting cells can restore or improve hearing and/or vestibular function.

In one aspect, the invention relates to a method of inducing proliferation or cell cycle reentry of a differentiated cochlear cell or a utricular cell. The method comprises increasing both c-myc activity and Notch activity within the cell sufficient to induce proliferation or cell cycle reentry of the cochlear cell or utricular cell. Upon entry into the cell cycle, the cell may dedifferentiate but retain aspects of its differentiated state. In certain embodiments, the cochlear or utricular cell can be, for example, a hair cell or a supporting cell. The method may also include the step of inhibiting c-myc and/or Notch activity after proliferation of the cochlear or the utricular hair or supporting cell to induce differentiation or transdifferentiation of the cell and/or at least one of its daughter cells into a hair cell. Inhibition of c-myc and/or Notch activity after proliferation can be important in promoting cell survival.

In another aspect, the invention relates to a method for regenerating a cochlear or utricular hair cell. The method includes increasing both c-myc activity and Notch activity within the hair cell thereby to induce cell proliferation to produce one, two or more daughter hair cells, and, after cell proliferation, decreasing c-myc and/or Notch activity to induce and/or maintain differentiation of the daughter hair cells. In certain embodiments, the cochlear or utricular cell can be, for example, a hair cell or a supporting cell. These steps can be performed in vivo (for example, in the inner ear of a mammal, in particular the cochlea or utricle), or ex vivo, wherein the resulting cells are cultured and/or introduced into the inner ear of a recipient.

In another aspect, the invention relates to a method for reducing the loss of, maintaining, or promoting hearing in a subject. The method comprises increasing both c-myc activity and Notch activity within a hair cell and/or a supporting cell of the inner ear thereby to induce cell proliferation to produce daughter cells, and, after cell proliferation, decreasing c-myc and/or Notch activity, and permitting daughter cells of hair cell origin to differentiate into hair cells or permitting daughter cells of supporting cell origin to transdifferentiate into hair cells thereby to reduce the loss of, maintain or promote hearing in the subject. The daughter cells of supporting cell origin can be induced to transdifferentiate into hair cells by activating Atoh1 activity, for example, by gene expression, by administration of an effective amount of Atoh1 or an Atoh1 agonist. The steps can be performed in vivo (for example, in the inner ear of a mammal, in particular in the cochlea), or ex vivo, wherein the resulting cells are cultured and/or introduced into the inner ear of the subject.

In another aspect, the invention relates to a method for reducing the loss of, maintaining, or promoting vestibular function in a subject. The method comprises increasing both c-myc activity and Notch activity within a hair cell and/or a supporting cell of the inner ear thereby to induce cell proliferation to produce daughter cells, and, after cell proliferation, decreasing c-myc and/or Notch activity, and permitting daughter cells of hair cell origin to differentiate into hair cells or permitting daughter cells of supporting cell origin to transdifferentiate into hair cells thereby to reduce the loss of, maintain or promote vestibular function in the subject. The daughter cells of supporting cell origin can be induced to transdifferentiate into hair cells by activating Atoh1 activity, for example, by gene expression, by administration of an effective amount of Atoh1 or an Atoh1 agonist. The steps can be performed in vivo (for example, in the inner ear of a mammal, in particular in the utricle), or ex vivo, wherein the resulting cells are cultured and/or introduced into the inner ear of the subject.

In each of the foregoing aspects of the invention, c-myc activity may be increased by contacting the cell with an effective amount of a c-myc protein or a c-myc activator. After c-myc activity is increased, c-myc activity can be inhibited to limit proliferation of the cochlear cell or utricular cell and/or to promote survival of the cochlear cell or utricular cell. Similarly, in each of the foregoing aspects of the invention, Notch activity may be increased by contacting the cell with an effective amount of a Notch protein, a Notch Intracellular Domain (NICD) protein or a Notch activator. Notch activity can be inhibited by contacting the cell with an effective amount of a Notch inhibitor.

In certain embodiments, the c-myc protein or c-myc activator may be administered to the inner ear of a subject. In certain embodiments, the Notch protein, NICD protein, Notch activator, and/or Notch inhibitor may be administered to the inner ear of a subject. In other embodiments, the c-myc protein or c-myc activator may be co-administered together with the Notch protein, the NICD protein, the Notch activator, and/or the Notch inhibitor to the inner ear of the subject.

The foregoing aspects and embodiments of the invention may be more fully understood by reference to the following figures, detailed description and claims.

### BRIEF DESCRIPTION OF THE DRAWINGS

The objects and features of the invention may be more fully understood by reference to the drawings described herein.

FIG. 1A shows the full-length protein sequence of human c-myc (NP_002458.2; SEQ ID NO: 1), and FIG. 1B shows the c-myc protein consensus protein sequence (SEQ ID NO: 9).

FIG. 2A shows the full-length protein sequence of human Notch (NP_060087.3; SEQ ID NO: 2), FIG. 2B shows the protein sequence of human Notch intracellular domain (NP_060087.3 residues 1754-2555; SEQ ID NO: 7), and FIG. 2C shows a consensus protein sequence of the Notch Intracellular domain (SEQ ID NO: 10).

FIG. 3A shows the full-length protein sequence of human Atoh1 (NP_005163.1; SEQ ID NO: 3) and FIG. 3B shows an Atoh1 consensus protein sequence (SEQ ID NO: 11).

FIG. 4 shows the nucleic acid sequence of human c-myc mRNA (NM_002467.4; SEQ ID NO: 4).

FIG. 5A shows the nucleic acid sequence of human Notch mRNA (NM_017617.3; SEQ ID NO: 5) and FIG. 5B shows the nucleotide sequence of human Notch intracellular domain (NM_017617.3 nucleotide positions 5260 to 7665; SEQ ID NO: 8).

FIG. 6 shows the nucleic acid sequence of human Atoh1 mRNA (NM_005172.1; SEQ ID NO: 6).

FIG. 7 shows cochlear hair and supporting cells double-labeled with cell-type specific markers and BrdU 4 days (panels A-E), 8 days (panels K-O), or 12 days (panels P-T) post-injection of Ad-Cre-GFP virus and Ad-Myc virus into cochleas of 45-day-old NICD^{flox/flox} mice. Solid arrows indicate BrdU labeled hair cells and open arrows indicate BrdU labeled supporting cells. Panels F-J show an uninjected control cochlea in which no hair and supporting cells double-labeled with cell-type specific markers and BrdU could be found. Panels A, F, K, and P show BrdU labeling. Panels B, G, L, and Q show Myo7a labeling of hair cells. Panels C, H, M, and R show Sox2 labeling of supporting cells. Panels D, I, N, and S show DAPI labeling of cell nuclei. Panels E, J, O, and T show merged images.

FIG. 8 shows cochlear hair and supporting cells double-labeled with cell-type specific markers and BrdU in the cochlear epithelium of NICD^{flox/flox} mice 35 days post-injection of an Ad-Cre-GFP/Ad-Myc mixture followed by 5 days of daily BrdU administration. Panels A, F, and K show BrdU labeling. Panels B, G, and L show Myo7a labeling of hair cells. Panels C, H, and M show Sox2 labeling of supporting cells. Panels D, I, and N show DAPI labeling of cell nuclei. Panels E, J, and O show merged images. Panels A-E show labeling with BrdU and Myo7a, demonstrating that proliferating hair cells survive 35 days post-injection (solid arrows, panels A, B, C, and E). panels F-J show an enlarged image of two hair cells displaying stereocilia (solid arrowhead, Panel J) derived from division of one mother hair cell. Panels K-O show cells labeled with BrdU and Sox2 (open arrows, panels K, M, and O), demonstrating that proliferating supporting cells survive 35 days post-injection. Closed arrows in panels K, L, M, and O show Myo7a+/BrdU+ hair cells. Arrowhead in panels K,L,M, and O show Myo7a+/Sox2+/BrdU+ hair cell.

FIG. 9 shows cochlear hair and supporting cells double-labeled with cell-type specific markers and BrdU in the cochlear epithelium of aged NICD^{flox/flox} mice injected with an Ad-Cre-GFP/Ad-Myc mixture over the course of 15 days. Panels A, F, and K show Myo7a labeling of hair cells. Panels B, G, and L show BrdU labeling of dividing cells. Panels C, H, and M show Sox2 labeling of supporting cells. Panels D, I, and N show DAPI labeling of cell nuclei. Panels E, J, and O show merged images. Panels A-J show Myo7a+/BrdU+ hair cells (panels A, B, and E; arrows) and Sox2+/BrdU+ supporting cells (panels B, C, E, G, H, and J; arrowheads) following injection with Ad-Myc and Ad-Cre-GFP adenovirus. Panels K-O show the same staining in 17-month old NICD^{flox/flox} mice injected with Ad-Cre-GFP virus alone. No BrdU labeled hair cells or supporting cells were found in the latter group. Scale bars: 10 μM.

FIG. 10 shows BrdU (panels A and F), Myo7a (panels B and G) and Sox2 (panels C and H) labeled hair and supporting cells in cultured adult human cochlear (panels A-E) and utricular (panels F-J) tissue transduced with Ad-Myc/Ad-NICD for 10 days. Open arrows (panels A, C, D, E, F, H, I, and J) indicate proliferating supporting cells (Sox2+/BrdU+) and solid arrow (panels F-J) indicates a proliferating hair cell (Myo7a+/BrdU+). Nuclear staining is shown by DAPI (D and I).

FIG. 11 shows Myo7+ hair (panels A and F) and Sox2+ supporting (panels C and H) cells in adult monkey cochlear cultures. Dividing cells were labeled with EdU (panels B and G). Panels A-E show Ad-GFP infected control monkey cochlea, in which no EdU+ cells were identified. Panels G, H, and J show EdU+/Sox2+ supporting cells (arrowheads) in monkey cochlea cultures exposed to Ad-Myc/Ad-NICD virus. In both control and Ad-Myc/Ad-NICD virus infected cultures, no hair cells were observed to re-enter the cell cycle (panels A, E, F, and J; arrows). Scale bars: 20 μM.

FIG. 12 shows selective induction of proliferation in supporting cells (arrows; panels B, C, and E), but not inner hair cells (arrowheads; panels A, C, and E), of rtTa/tet-on-Myc/tet-on-NICD mice exposed to doxycycline administered by an implanted osmotic pump for 9 days to induce expression of NICD and Myc. Cells that reentered the cell cycle were labeled via daily EdU (panel B) administration during the same period. Cell nuclei were stained for DAPI (panel D). Inner hair cells were stained for Parvalbumin (Parv; panel A). Supporting cells were stained for Sox2 (panel C). A single Parv+ hair cell is shown that also expressed Sox2 due to Notch activation (rightmost arrowhead in panels A, C, and E). Outer hair cells are not shown as they were lost during surgical implantation of the osmotic pump. Scale bar: 20 μM.

FIG. 13 shows outer hair cells are selectively induced to undergo cell cycle reentry following exposure to elevated c-Myc and Notch activity in vivo. rtTa/tet-on-Myc/tet-on-NICD mice were exposed to doxycycline administered by an implanted osmotic pump for 12 days to induce expression of NICD and Myc, after which tissue was harvested for staining. Cells that reentered the cell cycle were labeled via daily EdU (panel B) administration during the period of doxycycline exposure. Cell nuclei were stained for DAPI (panel D). Inner and outer hair cells were stained for Espin (Esp; panel A). Supporting cells were stained for Sox2 (panel C). Note that outer hair cells were spared during implantation of the osmotic pump in this experiment, as opposed to the experiment shown in FIG. 12. A dividing Esp+/EdU+ outer hair cell is shown in FIG. 13 (panels B and E; arrows), demonstrating selective induction of outer hair cell proliferation at this level of exposure to elevated c-Myc and Notch activity.

FIG. 14 shows Espin-positive (Esp+) hair cells labeled with FM-143FX (FM1) to reveal cells with functional membrane channels. Cochlea of 45-day-old NICDflovflex mice were exposed to Ad-Myc/Ad-Cre-GFP virus and EdU was injected once daily for 5 days following virus injection to label dividing cells. 35 days post-virus injection, cochlea were harvested, briefly exposed to FM1, fixed, and stained. Panels A-E show an Esp+/FM1+/EdU-control hair cell that has not undergone cell cycle reentry, but which expresses Esp and takes up FM1. Panels F-J show an Esp+/FM1+/EdU+ hair cell in a cochlea exposed to Ad-Myc/Ad-NICD virus, indicating the presence of functional membrane channels in a cell that has undergone cell cycle reentry. Arrowhead (panel H) indicates EdU labeling; arrow (panel F) indicates the presence of Esp+ hair bundles. Scale bars: 10 μM.

FIG. 15 shows that production of Myo7a+ hair cells induced to undergo cell

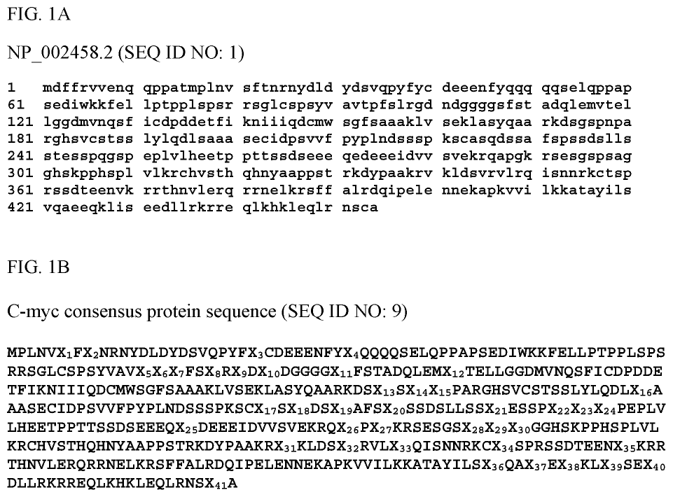

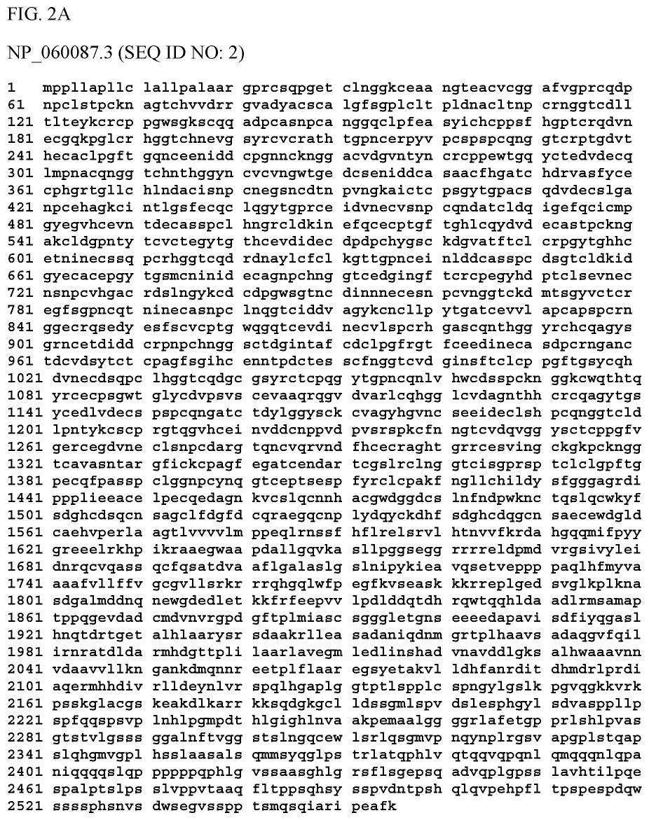

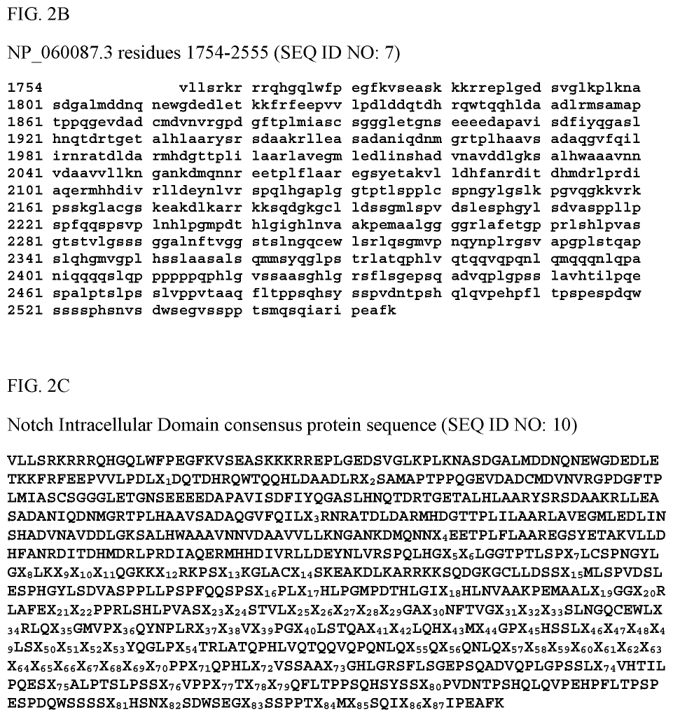

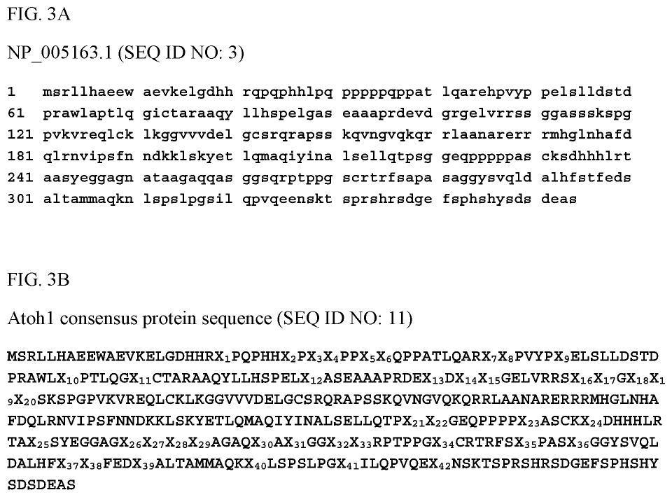

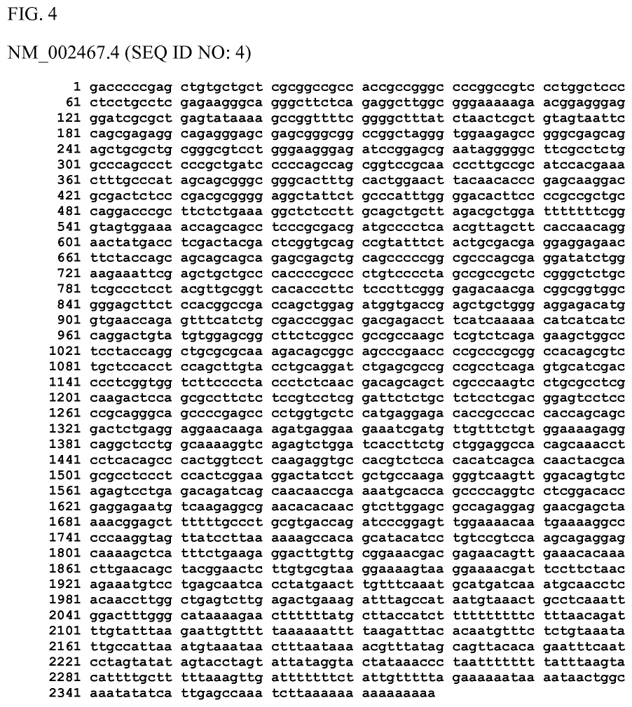

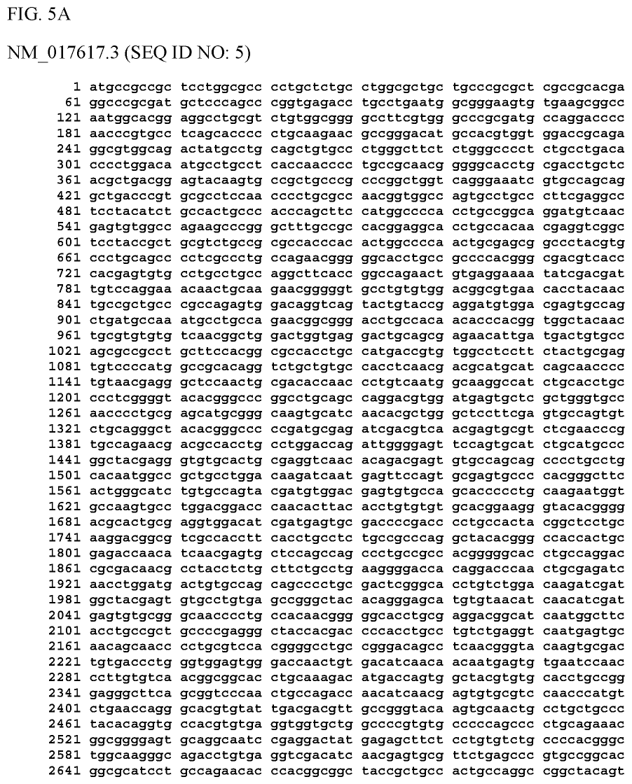

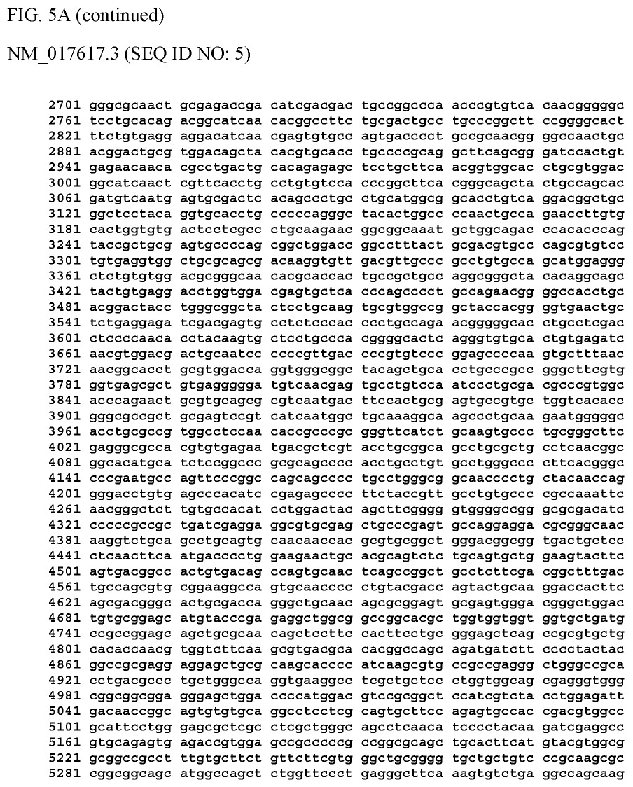

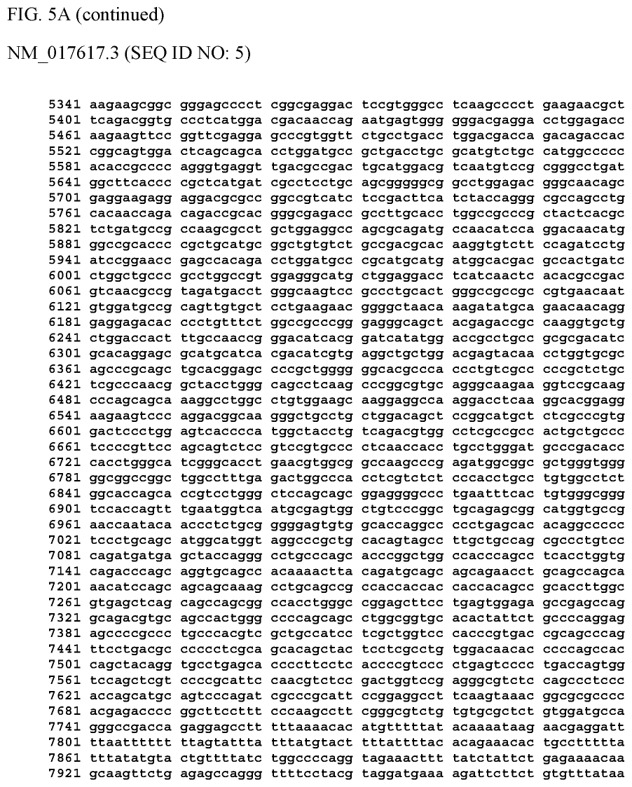

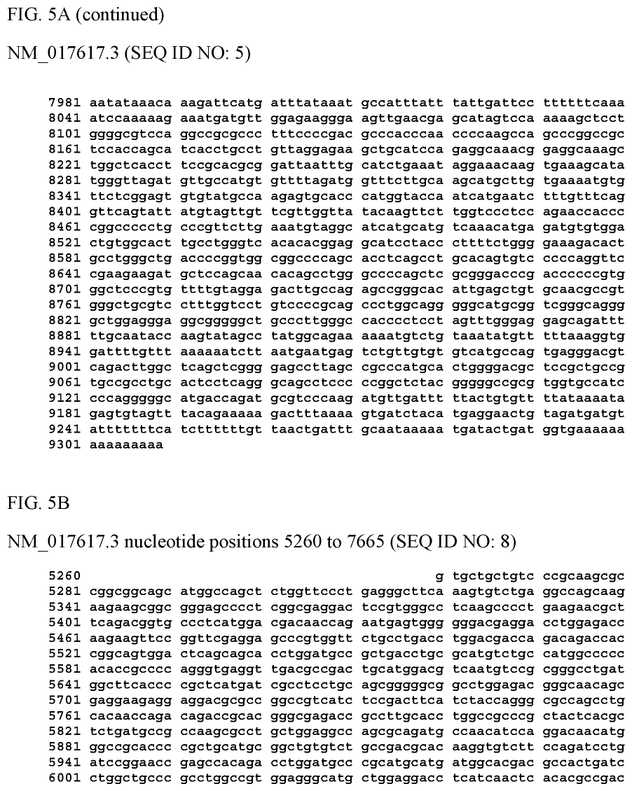

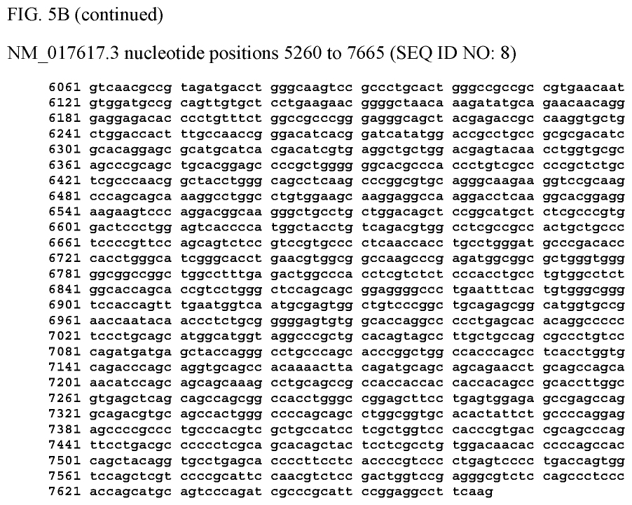

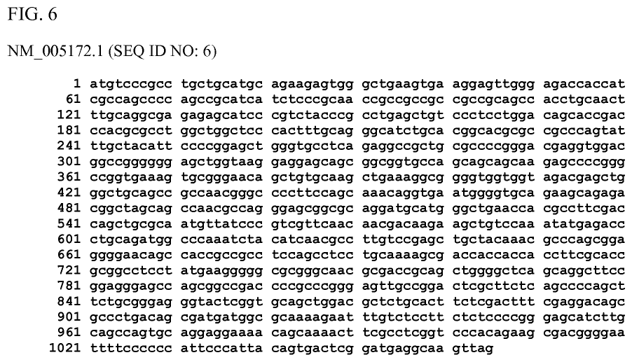

proliferation following exposure to elevated levels of c-Myc and Notch activity is accompanied by production of neurofilament-positive (NF+; panel B) neurofibers. Cochlea of 45-day-old NICD/^{flow/flox} mice were exposed to Ad-Myc/Ad-Cre-GFP virus and BrdU was injected once daily for 15 days following virus injection to label dividing cells (panel C). Tissue was harvested and stained 20 days post-virus injection. Panel A shows Myo7+ hair cells. Cell nuclei were stained using DAPI (panel D). Panel E shows a merge of all stains and an enlarged view of the boxed arca indicated by the rightmost arrow in the panel. Arrows (panels A, C, and E) indicate Myo7a+/BrdU+ hair cells in contact with NF+ ganglion neuron neurofibers. Scale bar: 10 μM.

FIG. 16 shows an example of an inner hair cell induced to proliferate via exposure to elevated levels of c-Myc and Notch activity and expressing an inner hair cell-specific marker (Vglut3; panels B and G) and a marker of functional synapses (CtBP2; panels A and F; brackets). Cochlea of 45-day-old NICD/^{flox/flox} mice were exposed to Ad-Myc/Ad-Cre-GFP (panels A-E) or Ad-GFP (panels F-J) virus via a single injection of virus, and BrdU was injected once daily for 15 days following virus injection to label dividing cells (panels C and H). Tissue was then harvested and stained. Cell nuclei were stained with DAPI (panels D and I). Panels A-E show a CtBP2+/VGlut3+/BrdU+ inner hair cell (panel B; arrow) induced to proliferate following exposure to elevated c-Myc and Notch activity, and a CtBP2+/Vglut3+/BrdU-inner hair cell (panel B; arrowhead) that did not undergo cell cycle reentry. Panels F-J show inner hair cells exposed to Ad-GFP that did not stain positive for BrdU but expressed the inner hair cell-specific marker Vglut3 and the presynaptic marker CtBP2. IHC=inner hair cell layer.

FIG. 17 shows cultured cochlear support cells from doxycycline-inducible rtTa/tet-on-Myc/tet-on-Notch mice induced to transdifferentiate or proliferate and transdifferentiate to functional hair cells following exposure to either Atoh1-expressing adenovirus alone (panels F-J) or doxycycline and Atoh1-expressing adenovirus (Ad-Atoh1; panels A-E and K-O). Cochlea from adult rtTa/tet-on-Myc/tet-on-Notch mice were dissected and cultured for 5 days in the presence (panels A-E and K-O) or absence (panels F-J) of doxycycline, followed by Ad-Atoh1 infection and an additional 14 days of culture. EdU was added daily to label dividing cells (panels A, F, and M). Cell nuclei were stained with DAPI (panels D, I, and N). Panels A-E show supporting cells exposed to doxycycline followed by Ad-Atoh1, and labeled with EdU, reenter the cell cycle and/or transdifferentiate into Myo7a+/Parv+ hair cells (closed arrows in panels A, B, C, and E). Open arrow in panels B, C, and E indicates the presence of a Myo7a+/Parv+ supporting cell that has transdifferentiated into a hair cell, but has not undergone cell cycle reentry. Arrowhead in panels A and E indicates an EdU+ supporting cell. Panels F-J shows supporting cells exposed to Ad-Atoh1, but not doxycycline, transdifferentiate to Myo7a+/Parv+ hair cells. Arrow in panels G, H, and J indicates a supporting cell that has transdifferentiated into a Myo7a+/Parv+ hair cell, but which has not undergone cell cycle reentry. Panels K-O show supporting cells exposed to doxycycline followed by Ad-Atoh1 and labeled with FMI (panel L) and Edu (panel M) have Esp+ hair bundles (panel K) and take up FMI dye. Arrow in panels K and O indicates an Esp+/FM1+/EdU+ hair cell displaying stereocilia derived from a transdifferentiated supporting cell that has undergone cell cycle reentry. Arrowhead in panels K and O indicates an Esp+/FM1+/EdU-hair cell derived from a transdifferentiated supporting cell that has not undergone cell cycle reentry. Scale bar: 10 μM.

FIG. 18 shows the results of semi-quantitative RT-PCR analysis of sets of mRNA transcripts produced in control cochlear cells and in cochlear cells following exposure to elevated c-Myc and NICD levels. Adult NICDflovflox mouse cochleas were exposed to Ad-Myc/Ad-Cre-GFP (Myc+Nicd) or Ad-GFP (Ctr) and cultured for 4 days, mRNA was extracted, and semi-quantitative RT-PCT was performed. Changes in expression of stem cell genes (Nanog, ALPL, and SSEA) and ear progenitor cell genes/Notch genes (Eya1, DLX5, Six1, Pax2, p27kip1, Islet-1, Sox2, Math1, NICD, Prox1, and Hes5) was examined. GAPDH expression was used as an internal control.

FIG. 19A shows the full-length protein sequence of human N-myc (NP_005369.2;SEQ ID NO: 12) and FIG. 19B shows the nucleic acid sequence of human N-myc (NM_005378.4; SEQ ID NO: 13).

FIG. 20A shows the full-length protein sequence of human Notch2 (NP_077719.2; SEQ ID NO: 14) and FIG. 20B shows the nucleic acid sequence of human Notch2 (NM_024408.3; SEQ ID NO: 15).

FIG. 21A shows the full-length protein sequence of human Notch3 (NP_000426.2; SEQ ID NO: 16) and FIG. 21B shows the nucleic acid sequence of human Notch3 (NM_000435.2; SEQ ID NO: 17).

FIG. 22A shows the full-length protein sequence of human Notch4 (NP_004548.3; SEQ ID NO: 18) and FIG. 22B shows the nucleic acid sequence of human Notch4 (NM_004557.3; SEQ ID NO: 19).

FIG. 23A shows the full-length protein sequence of human Atoh7 (NP_660161.1; SEQ ID NO: 20) and FIG. 23B shows the nucleic acid sequence of human Atoh7 (NM_145178.3; SEQ ID NO: 21).

FIG. 24 shows the nucleic acid sequence for an Atoh1 enhancer (SEQ ID NO: 22), which controls expression in hair cells.

FIG. 25 shows the nucleic acid sequence for a Pou4f3 promoter (SEQ ID NO: 23), which controls expression in hair cells.

FIG. 26 shows the nucleic acid sequence for a Myo7a promoter (SEQ ID NO: 24), which controls expression in hair cells.

FIG. 27 shows the nucleic acid sequence for a Hes5 promoter (SEQ ID NO: 25), which controls expression in vestibular supporting cells and cochlear inner phalangeal cells, Deiters cells and Pillar cells.

FIG. 28 shows the nucleic acid sequence for a GFAP promoter (SEQ ID NO: 26), which controls expression in vestibular supporting cells and cochlear inner phalangeal cells, Deiters cells and Pillar cells.

### DETAILED DESCRIPTION

The invention relates to methods and compositions for inducing cell cycle reentry and proliferation of hair and/or supporting cells in the ear, in particular, the inner ear. The methods and compositions can be used to increase a population of hair cells and/or supporting cells diminished by environmental or genetic factors. Using the methods and compositions described herein, it may be possible to preserve or improve hearing and/or vestibular function in the inner ear.

As demonstrated herein, simultaneously increasing c-myc and Notch activity appears to be an important step in inducing cell cycle reentry and proliferation in cells of the inner ear. As shown in the Examples below, overexpression of c-myc and Notch in the inner ear of a mammal results in the reentry of hair and supporting cells into the cell cycle and the proliferation of those cells. The proliferation of hair cells (or the proliferation of supporting cells followed by transdifferentiation of those cells into hair cells) may lead to improved hearing and/or vestibular function in a subject.

### Definitions

For convenience, certain terms in the specification, examples, and appended claims are collected in this section.

As used herein, the term “effective amount” is understood to mean the amount of an active agent, for example, a c-myc or Notch activator, that is sufficient to induce cell cycle reentry and/or proliferation of the cells of the inner ear (e.g., a hair cell or a supporting cell). The cells are contacted with amounts of the active agent effective to induce cell cycle reentry and/or proliferation.

As used herein, “pharmaceutically acceptable” or “pharmacologically acceptable” mean molecular entities and compositions that do not produce an adverse, allergic or other untoward reaction when administered to an animal, or to a human, as appropriate. The term, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The term “subject” is used throughout the specification to describe an animal, human or non-human, to whom treatment according to the methods of the present invention is provided. Veterinary and non-veterinary applications are contemplated. The term includes, but is not limited to, birds and mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

As used herein “target cell” and “target cells” refers to a cell or cells that are capable of reentering the cell cycle and/or proliferating and/or transdifferentiating to or towards a cell or cells that have or result in having characteristics of auditory or vestibular hair cells. Target cells include, but are not limited to, e.g., hair cells, e.g., inner ear hair cells, which includes auditory hair cells (inner and outer hair cells) and vestibular hair cells (located in the utricle, saccule and three semi-circular canals, for example), progenitor cells (e.g., inner ear progenitor cells), supporting cells (e.g., Deiters' cells, pillar cells, inner phalangeal cells, tectal cells and Hensen's cells), supporting cells expressing one or more of p27_{kip,} p75, S100A, Jagged-1, Prox1, and/or germ cells. “Inner hair cell” refers to a sensory cell of the inner ear that is anatomically situated in the organ of Corti above the basilar membrane. “Outer hair cell” refers to a sensory cell of the inner ear that is anatomically situated in the organ of Corti below the tectorial membrane near the center of the basilar membrane. Examples of target cells also include fibrocytes, marginal cells or interdental cells expressing one or more of Gjb2, Slc26a4 and Gjb6. As described herein, prior to treatment with the methods, compounds, and compositions described herein, each of these target cells can be identified using a defined set of one or more markers (e.g., cell surface markers) that is unique to the target cell. A different set of one or more markers (e.g., cell surface markers) can also be used to identify target cells have characteristics of an auditory hair cell or supporting cell.

As used herein, the term “host cell” refers to cells transfected, infected, or transduced in vivo, ex vivo, or in vitro with a recombinant vector or a polynucleotide. Host cells may include packaging cells, producer cells, and cells infected with viral vectors. In particular embodiments, host cells infected with viral vector of the invention are administered to a subject in need of therapy. In certain embodiments, the term “target cell” is used interchangeably with host cell and refers to transfected, infected, or transduced cells of a desired cell type.

The term “vector” is used herein to refer to a nucleic acid molecule capable transferring or transporting another nucleic acid molecule. The transferred nucleic acid is generally linked to, for example, inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial or yeast artificial chromosomes, and viral vectors. Useful viral vectors include, for example, adenoviruses, replication defective retroviruses, and lentiviruses.

As used herein, the term “viral vector” refers either to a nucleic acid molecule that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term “viral vector” may also refer either to a virus or viral particle capable of transferring a nucleic acid into a cell or to the transferred nucleic acid itself. Viral vectors and transfer plasmids contain structural and/or functional genetic elements that are primarily derived from a virus.

The term “retroviral vector” refers to a viral vector or plasmid containing structural and functional genetic elements, or portions thereof, that are primarily derived from a retrovirus.

The term “lentiviral vector” refers to a viral vector or plasmid containing structural and functional genetic elements, or portions thereof, that are primarily derived from a lentivirus.

The terms “lentiviral vector” or “lentiviral expression vector” may be used to refer to lentiviral transfer plasmids and/or infectious lentiviral particles. It is understood that nucleic acid sequence elements such as cloning sites, promoters, regulatory elements, heterologous nucleic acids, etc., are present in RNA form in the lentiviral particles of the invention and are present in DNA form in the DNA plasmids of the invention.

The term “hybrid” refers to a vector, LTR or other nucleic acid containing both retroviral (e.g., lentiviral) sequences and non-lentiviral viral sequences. A hybrid vector may refer to a vector or transfer plasmid comprising retroviral (e.g., lentiviral) sequences for reverse transcription, replication, integration and/or packaging. In some embodiments of the invention, a hybrid vector may be used to practice the invention described herein.

The term “transduction” refers to the delivery of a gene(s) or other polynucleotide sequence using a retroviral or lentiviral vector by means of viral infection rather than by transfection. In certain embodiments, a cell (e.g., a target cell) is “transduced” if it comprises a gene or other polynucleotide sequence delivered to the cell by infection using a viral (e.g., adenoviral) or retroviral vector. In particular embodiments, a transduced cell comprises one or more genes or other polynucleotide sequences delivered by a retroviral or lentiviral vector in its cellular genome.

As used herein, the term “c-myc” refers to a multifunctional, nuclear phosphoprotein that plays a role in cell cycle progression, apoptosis and cellular transformation and/or has an amino sequence or consensus amino acid sequence set forth in Section 1 (i) below. The full length sequence of human c-myc appears, for example, in the NCBI protein database under accession no. NP_002458.2 (sec ncbi.nlm.nih.gov and SEQ ID NO: 1). A consensus sequence for c-myc built from an alignment of human, rat, mouse and chimpanzee using ClustalW is set forth in SEQ ID NO: 9. C-myc functions as a transcription factor that regulates transcription of specific target genes. Mutations, overexpression, rearrangement and translocation of this gene have been associated with a variety of hematopoietic tumors, leukemias and lymphomas, including Burkitt lymphoma. C-myc is also known in the art as MYC, v-myc myelocytomatosis viral oncogene homolog (avian), transcription factor p64, bHLHe39, MRTL, avian myelocytomatosis viral oncogene homolog, v-myc avian myelocytomatosis viral oncogene homolog, myc proto-oncogene protein, class E basic helix-loop-helix protein 39, myc-related translation/localization regulatory factor, and proto-oncogene c-Myc, and BHLHE39.

As used herein, the term, “Notch” refers to the Notch family of signaling proteins, which includes Notch1, Notch2, Notch3 and Notch4, a NICD, and/or a protein having an amino acid sequence or consensus amino acid sequence set forth in Section (1)(i) below. The full length sequence of human Notch1 appears, for example, in the NCBI protein database under accession no. NP_060087.3 (see ncbi.nlm.nih.gov and SEQ ID NO: 2). Members of this Type 1transmembrane protein family share structural characteristics including an extracellular domain consisting of multiple epidermal growth factor-like (EGF) repeats, and an intracellular domain consisting of multiple, different domain types. Notch family members play a role in a variety of developmental processes by controlling cell fate decisions.

Notch 1 is cleaved in the trans-Golgi network, and presented on the cell surface as a heterodimer. Notch1 functions as a receptor for membrane bound ligands Jagged1, Jagged2 and Deltal to regulate cell-fate determination. Upon ligand activation through the released notch intracellular domain (NICD) it forms a transcriptional activator complex with RBPJ/RBPSUH and activates genes of the enhancer of split locus. Notch 1 affects the implementation of differentiation, proliferation and apoptotic programs.

Disclosed herein is a method of inducing proliferation or cell cycle reentry of a differentiated cochlear cell or a utricular cell. The method comprises increasing c-myc, Notch or both c-myc activity and Notch activity within the cell sufficient to induce proliferation or cell cycle reentry of the cochlear cell or utricular cell.

In certain embodiments, the method includes increasing c-myc activity within a cell when Notch activity is already increased, for example, when Notch1 has been upregulated in response to damage to the inner ear. In certain embodiments, the invention relates to a method of inducing proliferation or cell cycle reentry of a differentiated cochlear cell or a utricular cell in which Notch activity is increased in response to damage to the cochlear cell or utricular cell, as compared to the level of Notch activity in undamaged cochlear cells or utricular cells, respectively. The method comprises increasing c-myc activity within the cochlear cell or utricular cell sufficient to induce proliferation or cell cycle reentry of the cochlear cell or utricular cell.

In other embodiments, the method includes increasing Notch activity within a cell, when c-myc activity is already increased. (See, for example, Lee et al. (2008) ASSOC. RES. OTOLARYNGOL. ABS.: 762.) In particular, the invention relates to a method of inducing proliferation or cell cycle reentry of a differentiated cochlear cell or a utricular cell in which c-myc activity is increased in response to damage to the cochlear cell or utricular cell, as compared to the level of c-myc activity in undamaged cochlear cells or utricular cells, respectively. The method comprises increasing Notch activity within the cochlear cell or utricular cell sufficient to induce proliferation or cell cycle reentry of the cochlear cell or utricular cell.

After c-myc activity, Notch activity, or both c-myc and Notch activities, as appropriate, is or are increased, Notch may be inhibited according to methods known in the art and/or described herein to cause proliferating supporting cells to transdifferentiate into hair cells. Alternatively, or in addition, after c-myc activity, Notch activity, or both c-myc and Notch activity is or are increased, as appropriate, Atoh1 activity can be increased to cause proliferating supporting cells to transdifferentiate into hair cells. Methods of increasing Atoh1 activity (including use of Atoh1 agonists) are known in the art (see, for example, U.S. Pat. No. 8,188,131; U.S. Patent Publication No. 20110305674; U.S. Patent Publication No. 20090232780; Kwan eta al. (f2009) INT'L SYMPOSIUM ON OFACTION AND TASTE: ANN. N.Y. ACAD. SCI. 1170:28-33; Daudet et al. (2009) DEV. BIO. 326:86-100; Takebayashi et al. (2007) DEV. BIO. 307:165-178; and Ahmed et al. (2012) DE. CELL 22(2):377-390.)

Also disclosed is a method of regenerating a cochlear or utricular hair cell. The method includes (a) increasing c-myc, Notch, or both c-myc activity and Notch activity, as appropriate, within the hair cell thereby to induce cell proliferation to produce one, two or more daughter cells, and (b) after cell proliferation, decreasing Notch activity to induce differentiation of at least one of the cell and the daughter cells to produce a differentiated cochlear or utricular hair cell. The process can occur in vivo or ex vivo. In one embodiment, Notch activity is decreased in a cell that originated from a supporting cell to cause the supporting cell to transdifferentiate into a hair cell. In another embodiment, Atoh1 activity is increased in a cell that originated from a supporting cell to cause the supporting cell to transdifferentiate into a hair cell.

In certain embodiments, after c-myc and Notch induce proliferation within a hair cell or supporting cell, c-myc activity is decreased to induce differentiation of at least one of the cell and the daughter cell to produce a differentiated cochlear or utricular hair cell. Decreasing c-myc activity after proliferation can promote survival of the proliferating cell.

Also disclosed is a method for reducing the loss of, maintaining, or promoting hearing in a subject. The method comprises increasing c-myc activity, Notch activity, or both c-myc activity and Notch activity, as appropriate, within a hair cell and/or a supporting cell of the inner car thereby to induce cell proliferation to produce daughter cells, and, after cell proliferation, decreasing c-myc and/or Notch activity, and permitting daughter cells of hair cell origin to differentiate into hair cells or permitting daughter cells of supporting cell origin to transdifferentiate into hair cells thereby to reduce the loss of, maintain or promote hearing in the subject. The daughter cells of supporting cell origin can be induced to transdifferentiate into hair cells by activating Atoh1 or an Atoh1 agonist. The steps can be performed in vivo (for example, in the inner ear of a mammal, in particular in the cochlea), or ex vivo, wherein the resulting cells are cultured and/or introduced into the inner car of the subject.

Also disclosed is a method for reducing the loss of, maintaining, or promoting vestibular function in a subject. The method comprises increasing c-myc activity, Notch activity, or both c-myc activity and Notch activity, as appropriate, within a hair cell and/or a supporting cell of the inner ear thereby to induce cell proliferation to produce daughter cells, and, after cell proliferation, decreasing c-myc and/or Notch activity, and permitting daughter cells of hair cell origin to differentiate into hair cells or permitting daughter cells of supporting cell origin to transdifferentiate into hair cells thereby to reduce the loss of, maintain or promote vestibular function in the subject. The daughter cells of supporting cell origin can be induced to transdifferentiate into hair cells by activating Atoh1 activity, for example, by gene expression, by administration of an effective amount of Atoh1 or an Atoh1 agonist. The steps can be performed in vivo (for example, in the inner ear of a mammal, in particular in the utricle), or ex vivo, wherein the resulting cells are cultured and/or introduced into the inner ear of the subject.

The methods and compositions described herein can be used for treating subjects who have, or who are at risk for developing, an auditory disorder resulting from a loss of auditory hair cells, e.g., sensorineural hair cell loss. Patients having an auditory disorder can be identified using standard hearing tests known in the art. The method can comprise (a) increasing c-myc activity, Notch activity, or both c-myc activity and Notch activity, as appropriate, within the hair cell of the subject thereby to induce cell proliferation to produce a daughter cell, and (b) after cell proliferation, decreasing Notch activity to induce differentiation of at least one of the cell and the daughter cell to produce a differentiated cochlear or utricular hair cell. This can be accomplished by administering an agent or agents to the subject to modulate c-myc and Notch activity. Alternatively, the process can occur in cells (e.g., cochlear and/or utricular cells) ex vivo, after which the resulting cells are transplanted into the inner ear of the subject. In certain embodiments, the methods and compositions described herein can be used to promote growth of neurites from the ganglion neurons of the inner ear. For example, the regeneration of hair cells may promote the growth of new neurites from ganglion neurons and formation of new synapses with the regenerated hair cells to transmit sound and balance signals from the hair cells to the brain.

In certain embodiments, the methods and compositions described herein can be used to promote growth of neurites from the ganglion neurons of the inner ear. For example, the regeneration of hair cells may promote the growth of new neurites from ganglion neurons and formation of new synapses with the regenerated hair cells to transmit sound and balance signals from the hair cells to the brain. In some embodiments, the methods and compositions described herein can be used to reestablish proper synaptic connections between hair cells and auditory neurons to treat, for example, auditory neuropathy.

Subjects with sensorineural hair cell loss experience the degeneration of cochlea hair cells, which frequently results in the loss of spiral ganglion neurons in regions of hair cell loss. Such subjects may also experience loss of supporting cells in the organ of Corti, and degeneration of the limbus, spiral ligament, and stria vascularis in the temporal bone material.

In certain embodiments, the present invention can be used to treat hair cell loss and any disorder that arises as a consequence of cell loss in the car, such as hearing impairments, deafness, vestibular disorders, tinnitus (sec, Kaltenbach et al. (2002) J NEUROPHYSIOL. 88(2):699-714s), and hyperacusis (Kujawa et al. (2009) J. NEUROSCI. 29(45):14077-14085), for example, by promoting differentiation (e.g., complete or partial differentiation) of one or more cells into one or more cells capable of functioning as sensory cells of the car, e.g., hair cells.

In certain embodiments, the subject can have sensorineural hearing loss, which results from damage or malfunction of the sensory part (the cochlea) or non-sensory part (the limbus, spiral ligament and stria vascularis) or the neural part (the auditory nerve) of the car, or conductive hearing loss, which is caused by blockage or damage in the outer and/or middle car. Alternatively or in addition, the subject can have mixed hearing loss caused by a problem in both the conductive pathway (in the outer or middle car) and in the nerve pathway (the inner ear). An example of a mixed hearing loss is a conductive loss due to a middle-car infection combined with a sensorineural loss due to damage associated with aging.

In certain embodiments, the subject may be deaf or have a hearing loss for any reason, or as a result of any type of event. For example, a subject may be deaf because of a genetic or congenital defect; for example, a human subject can have been deaf since birth, or can be deaf or hard-of-hearing as a result of a gradual loss of hearing due to a genetic or congenital defect. In another example, a human subject can be deaf or hard-of-hearing as a result of a traumatic event, such as a physical trauma to a structure of the car, or a sudden loud noise, or a prolonged exposure to loud noises. For example, prolonged exposures to concerts, airport runways, and construction areas can cause inner ear damage and subsequent hearing loss.

In certain embodiments, a subject can experience chemical-induced ototoxicity, wherein ototoxins include therapeutic drugs including antineoplastic agents, salicylates, quinines, and aminoglycoside antibiotics, contaminants in foods or medicinals, and environmental or industrial pollutants.

In certain embodiments, a subject can have a hearing disorder that results from aging. Alternatively or in addition, the subject can have tinnitus (characterized by ringing in the cars) or hyperacusis (heightened sensitivity to sound).

In addition, the methods and compositions described herein can be used to treat a subject having a vestibular dysfunction, including bilateral and unilateral vestibular dysfunction. Vestibular dysfunction is an inner ear dysfunction characterized by symptoms that include dizziness, imbalance, vertigo, nausea, and fuzzy vision and may be accompanied by hearing problems, fatigue and changes in cognitive functioning. Vestibular dysfunction can be the result of a genetic or congenital defect; an infection, such as a viral or bacterial infection; or an injury, such as a traumatic or nontraumatic injury. Vestibular dysfunction is most commonly tested by measuring individual symptoms of the disorder (e.g., vertigo, nausea, and fuzzy vision).

Alternatively or in addition, the methods and compositions described herein can be used prophylactically, such as to prevent, reduce or delay progression of hearing loss, deafness, or other auditory disorders associated with loss of inner ear function. For example, a composition containing one or more of the agents can be administered with (e.g., before, after or concurrently with) a second composition, such as an active agent that may affect hearing loss. Such ototoxic drugs include the antibiotics neomycin, kanamycin, amikacin, viomycin, gentamycin, tobramycin, erythromycin, vancomycin, and streptomycin; chemotherapeutics such as cisplatin; nonsteroidal anti-inflammatory drugs (NSAIDs) such as choline magnesium trisalicylate, diclofenac, diflunisal, fenoprofen, flurbiprofen, ibuprofen, indomethacin, ketoprofen, meclofenamate, nabumetone, naproxen, oxaprozin, phenylbutazone, piroxicam, salsalate, sulindac, and tolmetin; diuretics; salicylates such as aspirin; and certain malaria treatments such as quinine and chloroquine. For example, a human undergoing chemotherapy can be treated using the compounds and methods described herein. The chemotherapeutic agent cisplatin, for example, is known to cause hearing loss. Therefore, a composition containing one or more agents that increase the activity of c-myc and Notch can be administered with cisplatin therapy (e.g., before, after or concurrently with) to prevent or lessen the severity of the cisplatin side effect. Such a composition can be administered before, after and/or simultaneously with the second therapeutic agent. The two agents may be administered by different routes of administration.

In certain embodiments, the methods and compositions described herein can be used to increase the levels (e.g., protein levels) and/or activity (e.g., biological activity) of c-myc and Notch in cells (e.g., inner ear cells). Exemplary methods and compositions include, but are not limited to methods and compositions for increasing c-myc or Notch expression (e.g., transcription and/or translation) or levels (e.g., concentration) in cells. It is contemplated that such modulation can be achieved in hair cells and/or supporting cells in vivo and ex vivo.

### 1. Methods and Compositions for Increasing C-myc and Notch and Atoh1 Activity

### (i) C-myc, Notch, or Atoh1 Polypeptides

It is contemplated that c-myc, Notch, and Atoh1 proteins, including full length proteins, biologically active fragments, and homologs of c-myc and Notch can be introduced into target cells using techniques known in the art.

Exemplary c-myc polypeptides include, for example, NP_002458.2 (SEQ ID NO: 1), as referenced in the NCBI protein database. Exemplary Notch polypeptides include, for example, NP_060087.3 (SEQ ID NO: 2), as referenced in the NCBI protein database. Exemplary Atoh1 polypeptides include, for example, NP_005163.1 (SEQ ID NO: 3), as referenced in the NCBI protein database.

In certain embodiments, nucleic acid sequences encoding c-myc, Notch, and Atoh1 family members may be used in accordance with the methods described herein. Exemplary c-myc family members include N-myc, referenced in the NCBI protein database as NP_005369.2 (SEQ ID NO: 12). Exemplary Notch family members include Notch2, referenced in the NCBI protein database as NP_077719.2 (SEQ ID NO: 14); Notch3, referenced in the NCBI protein database as NP_000426.2 (SEQ ID NO: 16); and Notch4, referenced in the NCBI protein database as NP_004548.3 (SEQ ID NO: 18). Exemplary Atoh1 family members include Atoh7, referenced in the NCBI protein database as NP_660161.1 (SEQ ID NO: 20).

In certain embodiments, a protein sequence of the invention may comprise a consensus protein sequence or a nucleotide sequence encoding a consensus protein sequence. Consensus protein sequences of c-myc, Notch intracellular domain, and Atoh1 of the invention are set forth below.

A consensus protein sequence of c-myc built from human, mouse, rat and chimpanzee sequences using ClustalW is as follows:

MPLNVX₁X₂NRNYDLDYDSVQPYFX₃CDEEENFYX₄QQQQSELQPPAPSEDIW KKFELLPTPPLSPSRRSGLCSPSYVAVX₅X₆X₇FSX₈RX₉DX₁₀DGGGGX₁₁FSTADQLEMX₁₂TE LLGGDMVNQSFICDPDDETFIKNIIIQDCMWSGFSAAAKLVSEKLASYQAARKDSX₁₃SX₁₄ X₁₅PARGHSVCSTSSLYLQDLX₁₆AAASECIDPSVVFPYPLNDSSSPKSCX₁₇SX₁₈DSX₁₉AFSX₂oSSDSLLSSX₂₁ESSPX₂₂X₂₃X₂₄PEPLVLHEETPPTTSSDSEEEQX₂₅DEEEIDVVSVEKRQX₂₆PX ₂₇KRSESGSX₂₈X₂₉X₃₀GGHSKPPHSPLVLKRCHVSTHQHNYAAPPSTRKDYPAAKRX₃₁KLD SX₃₂RVLX₃₃QISNNRKCX₃₄SPRSSDTEENX₃₅KRRTHNVLERQRRNELKRSFFALRDQIPELE NNEKAPKVVILKKATAYILSX₃₆QAX₃₇EX₃₈KLX₃₉SEX₄₀DLLRKRREQLKHKLEQLRNSX₄₁A (SEQ ID NO: 9), wherein X₁ is S or N; X₂ is T or A; X₃ is Y or I; X₄ is Q or H; X₅ is T or A; X₆ is P or T; X₇ is S or a bond; X₈ is L or P; X₉ is G or E; X₁₀ is N or D; X₁₁ is S or N; X₁₂ is V or M; X₁₃ is G or T; X₁₄ is P or L; X₁₅ is N or S; X₁₆ is S or T; X₁₇ is P or A or T; X₁₈ is Q or S; X₁₉ is S or T; X₂₀ is P or S; X₂₁ is T or a bond; X₂₂ is Q or R; X₂₃ is A or G; X₂₄ is S or T; X₂₅ is E or D; X₂₆ is A or T or P; X₂₇ is G or A; X₂₈ is P or S; X₂₉ is P or S; X₃₀ is A or S; X₃₁ is V or A; X₃₂ V or G; X₃₃ is K or R; X₃₄ is T or S; X₃₅ is D or V; X₃₆ is V or I; X₃₇ is E or D; X₃₈ is Q or H; X₃₉ is T or I; X₄₀ is E or K; and X₄₁ is C or G.

A consensus protein sequence of the Notch intracellular domain build from human, rat and mouse sequences using ClustalW is as follows:

VLLSRKRRRQHGQLWFPEGFKVSEASKKKRREPLGEDSVGLKPLKNASDGAL MDDNQNEWGDEDLETKKFRFEEPVVLPDLX₁DQTDHRQWTQQHLDAADLRX₂SAMAPTP PQGEVDADCMDVNVRGPDGFTPLMIASCSGGGLETGNSEEEEDAPA VISDFIYQGASLHN QTDRTGETALHLAARYSRSDAAKRLLEASADANIQDNMGRTPLHAAVSADAQGVFQILX₃RNRATDLDARMHDGTTPLILAARLAVEGMLEDLINSHADVNAVDDLGKSALHWAAAVN NVDAAVVLLKNGANKDMQNNX₄EETPLFLAAREGSYETAKVLLDHFANRDITDHMDRLP RDIAQERMHHDIVRLLDEYNLVRSPQLHGX₅X₆LGGTPTLSPX₇LCSPNGYLGX₈LKX₉X₁₀X_{1 1}QGKKX₁₂RKPSX₁₃KGLACX₁₄SKEAKDLKARRKKSQDGKGCLLDSSX₁₅MLSPVDSLESPH GYLSDVASPPLLPSPFQQSPSX₁₆PLX₁₇HLPGMPDTHLGIX₁₈HLNVAAKPEMAALX₁₉GGX₂₀RLAFEX₂₁X₂₂PPRLSHLPVASX₂₃X₂₄STVLX₂₅X₂₆X₂₇X₂₈X₂₉GAX₃₀NFTVGX₃₁X₃₂X₃₃SLNGQC EWLX₃₄RLQX₃₅GMVPX₃₆QYNPLRX₃₇X₃₈VX₃₉PGX₄₀LSTQAX₄₁X₄₂LQHX₄₃MX₄₄GPX_{4S}HSSL X₄₆X₄₇X₄₈X₄₉LSX₅₀X₅₁X₅₂X₅₃YQGLPX₅₄TRLATQPHLVQTQQVQPQNLQX₅₅QX₅₆QNLQX₅₇X ₅₈X₅₉X₆₀X₆₁X₆₂X₆₃X₆₄X₆₅X₆₆X₆₇X₆₈X₆₉X₇₀PPX₇₁QPHLX₇₂VSSAAX₇₃GHLGRSFLSGEPSQADV QPLGPSSLX₇₄VHTILPQESX₇₅ALPTSLPSSX₇₆VPPX₇₇TX₇₈X₇₉QFLTPPSQHSYSSX₈₀PVDNTP SHQLQVPEHPFLTPSPESPDQWSSSSX₈₁HSNX₈₂SDWSEGX₈₃SSPPTX₈₄MX₈₅SQIX₈₆X₈₇IPEA FK (SEQ ID NO: 10), wherein X₁ is D or S; X₂ is M or V; X₃ is L or I; X₄ is K or R; X₅ is T or A; X₆ is A or P; X₇ is T or P; X₈ is S or N; X₉ is S or P; X₁₀ is A or G; X₁₁ is T or V; X₁₂ is A or V; X₁₃ is T or S; X₁₄ is G or S; X₁₅ is G or S; X₁₆ is M or V; X₁₇ is S or N; X₁₈ is S or G; X₁₉ is A or G; X₂₀ is S or G; X₂₁ is P or T; X₂₂ is P or G; X₂₃ is S or G; X₂₄ is A or T; X₂₅ is S or G; X₂₆ is T or S; X₂₇ is N or S; X₂₈ is G or S; X₂₉ is T or G; X₃₀ is M or L; X₃₁ is A or G; X₃₂ is P or S; X₃₃ is A or T; X₃₄ is Por S; X₃₅ is N or S; X₃₆ is S or N; X₃₇ is P or G; X₃₈ is G or S; X₃₉ is T or A; X₄₀ is T or P; X₄₁ is A or P; X₄₂ is G or S; X₄₃ is G or S; X₄₄ is M or V; X₄₅ is L or I; X₄₆ is S or A; X₄₇ is T or A; X₄₈ is N or S; X₄₉ is T or A; X₅₀ is P or Q; X₅₁ is M or I; X₅₂ is M or I; X₅₃ is S or a bond; X₅₄ is S or N; X₅₅ is L or I or M; X₅₆ is Q or P; X₅₇ is a bond or P; X₅₈ is a bond or A; X₅₉ is a bond or N; X₆₀ is a bond or I; X₆₁ is a bond or Q; X₆₂ is a bond or Q; X₆₃ is a bond or Q; X₆₄ is a bond or Q; X₆₅ is a bond or S; X₆₆ is a bond or L; X₆₇ is a bond or Q; X₆₈ is a bond or P; X₆₉ is a bond or P; X70 is a bond or P; X₇₁ is P or S; X₇₂ S or G; X₇₃ is N or S; X₇₄ is P or A; X₇₅ is Q or P; X₇₆ is M or L; X₇₇ is M or V; X₇₈ is T or A; X₇₉ is T or A; X₈₀ is S or a bond; X₈₁ is Por R; X₈₂ is I or V; X₈₃ is I or V; X₈₄ is T or S; X₈₅ is P or Q; X₈₆ is T or A; X₈₇ is H or R.

A consensus protein sequence of Atoh1 built from human, mouse and chimpanzee sequences using ClustalW is as follows:

MSRLLHAEEWAEVKELGDHHRX₁PQPHHX₂PX₃X₄PPX₅X₆QPPATLQARX₇X₈PV YPX₉ELSLLDSTDPRAWLX₁₀PTLQGX₁₁CTARAAQYLLHSPELX₁₂ASEAAAPRDEX₁₃DX₁₄X ₁₅GELVRRSX₁₆X₁₇GX₁₈X₁₉X₂₀SKSPGPVKVREQLCKLKGGVVVDELGCSRQRAPSSKQVNG VQKQRRLAANARERRRMHGLNHAFDQLRNVIPSFNNDKKLSKYETLQMAQIYINALSEL LQTPX₂₁X₂₂GEQPPPPX₂₃ASCKX₂₄DHHHLRTAX₂₅SYEGGAGX₂₆X₂₇X²⁸X₂₉AGAQX₃₀AX₃₁G GX₃₂X₃₃RPTPPGX₃₄CRTRFSX₃₅PASX₃₆GGYSVQLDALHFX₃₇X³⁸FEDX₃₉ALTAMMAQKX₄₀L SPSLPGX₄₁ILQPVQEX₄₂NSKTSPRSHRSDGEFSPHSHYSDSDEAS (SEQ ID NO: 11), wherein X₁ is Q or H; X₂ is L or V; X₃ is Q or a bond; X₄ is P or a bond; X₅ is P or a bond; X₆ is P or a bond; X₇ is E or D; X₈ is H or L; X₉ is Por A; X₁₀ is A or T; X₁₁ is I or L; X₁₂ is S or G; X₁₃ is V or A; X₁₄ is G or S; X₁₅ is R or Q; X₁₆ is S or G; X₁₇ is G or C; X₁₈ is A or G; X₁₉ is S or a bond; X₂₀ is S or L; X₂₁ is S or N; X₂₂ is G or V; X₂₃ is P or T; X²⁴ is S or N; X₂₅ is A or S; X₂₆ is A or N; X₂₇ is A or S; X₂₈ is T or A; X₂₉ is A or V; X₃₀ is Q or P; X₃₁ is S or P; X₃₂ is S or G; X₃₃ is Q or P; X₃₄ is S or P; X₃₅ is A or G; X₃₆ is A or S; X₃₇ is S or P; X₃₈ is T or A; X₃₉ is S or R; X₄₀ is N or D; X₄₁ is S or G; and X₄₂ is E or D.

As used herein, the term “Atoh1” refers to a protein belonging to the basic helix-loop-helix (BHLH) family of transcription factors that is involved in the formation of hair cells in an inner ear of a mammal, and/or is a protein having an amino sequence or consensus sequence as set forth herein.

The c-myc, Notch, or Atoh1 polypeptides can be used in combination with

compositions to enhance uptake of the polypeptides into biological cells. In certain embodiments, the Atoh1, c-myc, or Notch polypeptides can be mutated to include amino acid sequences that enhance uptake of the polypeptides into a biological cell. In certain embodiments, Atoh1, c-myc, or Notch polypeptides can be altered or mutated to increase the stability and/or activity of the polypeptide (e.g., c-myc, Notch or Atoh-1 point mutants). In certain embodiments, c-myc, Notch or Atoh1 polypeptides can be altered to increase nuclear translocation of the polypeptide. In certain embodiments, altered c-myc, Notch or Atoh1 polypeptides or biologically active fragments of c-myc, Notch, or Atoh1 retain at least 50%, 60%, 70%, 80%, 90%, or 95% of the biological activity of full length, wild type respective c-myc, Notch or Atoh1 protein in the species that is the same species as the subject that is or will be treated with the methods and compositions described herein.

In certain embodiments, c-myc polypeptides sequences can be 50%, 60%, 70%, 80%, 85%, 90%, 95%, 98%, 99%, or 100% identical to NP_002458.2 (SEQ ID NO.: 1). In certain embodiments, Notch polypeptides sequences are 50%, 60%, 70%, 80%, 85%, 90%, 95%, 98%, 99%, or 100% identical to NP_060087.3 (SEQ ID NO.: 2). In certain embodiments, Atoh1 polypeptides sequences can be 50%, 60%, 70%, 80%, 85%, 90%, 95%, 98%, 99%, or 100% identical to NP_005163.1 (SEQ ID NO.: 3). In certain embodiments, agents encoded by modified Atoh1, c-myc, or Notch nucleic acid sequences and Atoh1, c-myc, or Notch polypeptide sequences possess at least a portion of the activity (e.g., biological activity) of the molecules encoded by the corresponding, e.g., unmodified, full-length Atoh1, c-myc, or Notch nucleic acid sequences and Atoh1, c-myc, or Notch polypeptide sequences. For example, molecules encoded by modified Atoh1, c-myc, or Notch nucleic acid sequences and modified Atoh1, c-myc, or Notch polypeptides retain 50%, 60%, 70%, 80%, 85%, 90%, 95%, 98%, 99%, or 100% of the activity (e.g., biological activity) of the molecules encoded by the corresponding, e.g., unmodified, respective Atoh1, c-myc, or Notch nucleic acid sequences and/or full length Atoh1, c-myc, or Notch polypeptide sequences.

In certain embodiments, the c-myc protein of the invention comprises functional domains at least 50%, 60%, 70%, 80%, 85%, 90%, 95%, 98%, 99%, or 100% identical to a Myc-N domain comprising amino acid residues 16-360 of SEQ ID NO: 1, a helix-loop-helix domain comprising amino acid residues 370-426 of SEQ ID NO: 1, a Myc leucine zipper domain comprising amino acid residues 423-454 of SEQ ID NO: 1, and/or surrounding and/or intervening sequences of SEQ ID NO: 1. In certain embodiments, the Notch protein of the invention comprises functional domains at least 50%, 60%, 70%, 80%, 85%, 90%, 95%, 98%, 99%, or 100% identical to a Notch intracellular domain comprising amino acid residues 1754-2555 of SEQ ID NO: 2. In certain embodiments, the Atoh1 protein of the invention comprises functional domains at least 50%, 60%, 70%, 80%, 85%, 90%, 95%, 98%, 99%, or 100% identical to a basic helix-loop-helix domain comprising amino acids 158-214 of SEQ ID NO: 3, a helix-loop-helix domain comprising amino acids 172-216 of SEQ ID NO: 3, and/or surrounding and/or intervening sequences of SEQ ID NO: 3.

In certain embodiments, the c-myc and Notch proteins of the invention can be administered to cells as a single protein containing both c-myc and Notch (or active domains thereof), preferably separated by a cleavable linker. Examples of cleavable linkers are known in the art (see, e.g., U.S. Pat. Nos. 5,258,498 and 6,083,486.)

C-myc, Notch or Atoh1 levels (e.g., protein levels) and/or activity (e.g., biological activity) in target cells and/or in the nucleus of target cells can be assessed using standard methods such as Western Blotting, in situ hybridization, reverse transcriptase polymerase chain reaction, immunocytochemistry, viral titer detection, and genetic reporter assays. Increases in c-myc, Notch or Atoh1 levels (e.g., protein levels) and/or activity (e.g., biological activity) in target cells and/or in the nucleus of target cells can be assessed by comparing c-myc, Notch or Atoh1 evels and/or activity in a first cell sample or a standard with c-myc, Notch or Atoh1 levels and/or activity in a second cell sample, e.g., contacting the cell sample with an agent contemplated to increase c-myc, Notch or Atoh1 levels and/or activity.

Sequence identity may be determined in various ways that are within the skill in the art, e.g., using publicly available computer software such as BLAST, BLAST-2, ALIGN or Megalign (DNASTAR) software, which are used to perform sequence alignments and then calculate sequence identity. Exemplary software programs available from the National Center for Biotechnology Information (NCBI) on the website ncbi.nlm.nih.gov include blastp, blastn, blastx, tblastn and tblastx. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are used at the default settings. The default scoring matrix used by blastp, blastx, tblastn, and tblastx is the BLOSUM62 matrix (Henikoff et al., (1992) PROC. NATL. ACAD. SCI. USA 89:10915-10919). In one approach, the percent identity can be determined using the default parameters of blastp, version 2.2.26 available from the NCBI.

### (ii) DNA Encoding Atoh1, C-myc, or Notch

Atoh1, c-myc, or Notch can be expressed in target cells using one or more expression constructs known in the art. Such expression constructs include, but are not limited to, naked DNA, viral, and non-viral expression vectors. Exemplary c-myc nucleic acid sequences that may be expressed in target cells include, for example, NM_002467.4 (SEQ ID NO: 4), as referenced in the NCBI gene database. Exemplary Notch nucleic acid sequences that may be expressed include, for example, NM_017617.3 (SEQ ID NO: 5), as referenced in the NCBI gene database. Exemplary Atoh1 nucleic acid sequences that may be expressed in target cells include, for example, NM_005172.1 (SEQ ID NO: 6), as referenced in the NCBI gene database.

In certain embodiments, c-myc, Notch, and Atoh1 family members may be used. Exemplary c-myc family members include N-myc, referenced in the NCBI gene database as NM_005378.4 (SEQ ID NO: 13). Exemplary Notch family members include Notch2, referenced in the NCBI gene database as NM_024408.3 (SEQ ID NO: 15); Notch3, referenced in the NCBI gene database as NM_000435.2 (SEQ ID NO: 17); and Notch4, referenced in the NCBI gene database as NM_004557.3 (SEQ ID NO: 19). Exemplary Atoh1 family members include Atoh7, referenced in the NCBI gene database as NM_145178.3 (SEQ ID NO: 21).

In certain embodiments, DNA encoding c-myc, Notch or Atoh1 can be an unmodified wild type sequence. Alternatively, DNA encoding c-myc, Notch or Atoh1 can be modified using standard techniques. For example, DNA encoding c-myc, Notch or Atoh1 can be modified or mutated, e.g., to increase the stability of the DNA or resulting polypeptide. Polypeptides resulting from such altered DNAs should retain the biological activity of wild type c-myc, Notch or Atoh1. In certain embodiments, DNA encoding Atoh1, c-myc, or Notch can be altered to increase nuclear translocation of the resulting polypeptide. In certain embodiments, DNA encoding c-myc, Notch or Atoh1 can be modified using standard molecular biological techniques to include an additional DNA sequence that can encode one or more of, e.g., detectable polypeptides, signal peptides, and protease cleavage sites.

In certain embodiments, c-myc nucleic acid sequences can be 50%, 60%, 70%, 80%, 85%, 90%, 95%, 98%, 99%, or 100% identical to NM_002467.4 (SEQ ID NO: 4). In certain embodiments, Notch nucleic acid sequences are 50%, 60%, 70%, 80%, 85%, 90%, 95%, 98%, 99%, or 100% identical to NM_017617.3 (SEQ ID NO: 5). In certain embodiments, Atoh1 nucleic acid sequences are 50%, 60%, 70%, 80%, 85%, 90%, 95%, 98%, 99%, or 100% identical to NM_005172.1 (SEQ ID NO: 6).

In certain embodiments, the c-myc nucleic acid sequence of the invention comprises functional domains at least 50%, 60%, 70%, 80%, 85%, 90%, 95%, 98%, 99%, or 100% identical to DNA encoding a Myc-N domain comprising amino acid residues 16-360 of SEQ ID NO: 1, a helix-loop-helix domain comprising amino acid residues 370-426 of SEQ ID NO: 1, DNA encoding a Myc leucine zipper domain comprising amino acid residues 423-454 of SEQ ID NO: 1,and/or DNA encoding the surrounding and/or intervening sequences of SEQ ID NO: 1. In certain embodiments, the Notch nucleic acid sequence of the invention comprises functional domains at least 50%, 60%, 70%, 80%, 85%, 90%, 95%, 98%, 99%, or 100% identical to DNA encoding a Notch intracellular domain comprising amino acid residues 1754-2555 of SEQ ID NO: 2. In certain embodiments, the Atoh1 nucleic acid sequence of the invention comprises functional domains at least 50%, 60%, 70%, 80%, 85%, 90%, 95%, 98%, 99%, or 100% identical to DNA encoding a basic helix-loop-helix domain comprising amino acids 158-214 of SEQ ID NO: 3,DNA encoding a helix-loop-helix domain comprising amino acids 172-216 of SEQ ID NO: 3,and/or DNA encoding surrounding and/or intervening sequences of SEQ ID NO: 3.

(iii) C-myc, Notch or Atoh1 Pathway Modulators

In certain embodiments, c-myc or Notch levels (e.g., protein levels) and/or activity (e.g., biological activity) can be increased or decreased using compounds or compositions that target c-myc or Notch, or one or more components of the c-myc or Notch pathway. Similarly, Atoh1 levels (e.g., protein levels) and/or activity (e.g., biological activity) can be increased using compounds that target Atoh1 or one or more components of the Atoh1 pathway.

Exemplary c-myc activators include microRNAs that target FBXW-7 (Ishikawa Y et al., Oncogene 2012 Jun. 4; doi:10.1038/onc.2012.213) and activators that increase c-myc expression levels or activity such as nordihydroguaiaretic acid (NDGA) (Park S et al. (2004) J. CELL BIOCHEM. 91(5):973-86), CD19 (Chung et a.l, (2012) J. CLIN. INVEST. 122(6):2257-2266, cohesin (McEwan et al. (2012) PLOS ONE 7(11): c49160), bryostatin 1 (Hu et al. (1993) LEUK. LYMPHOMA 10(1-2):135-42), 2′-3-dimethyl-4-aminoazobenzene (ortho-aminoazotoluene, OAT) (Smetanina et al. (2011) TOXICOL. APPL. PHARMACOL. 255(1):76-85), 2-amino-1-methyl-6-phenylimidazo[4,5-b]pyridine (PhIP) (Lauber et al. (2004) CARCINOGENESIS 25(12):2509-17), β-estradiol ( 7,544,511 B2), RU38486 (U.S. Pat. No. 7,544,511B2), dexamethasone (U.S. Pat. No. 7,544,511B2), thyroid hormones (U.S. Pat. No. 7,544,511B2), retinoids (U.S. Pat. No. 7,544,511B2), and ecdysone (U.S. Pat. No. 7,544,511 B2).

Exemplary c-myc inhibitors include 7-nitro-N-(2-phenylphenyl)-2,1,3-benzoxadiazol-4-amine (10074-G5) (Clausen D M et al., (2010) J. PHARMACOL. EXP. THER. 335(3):715-27), thioxothiazolidinone [Z-E]-5-[4-ethylbenzylidene]-2-thioxo-1,3-thiazolidin-4-one (10058-F4) (Clausen et al. (2010) J. PHARMACOL. EXP. THER. 335(3):715-27; Lin C P et al. (2007) ANTICANCER DRUGS. 18(2): 161-70; Huang et al. (2006) EXP. HEMATOL. 34(11):1480-9), 4-phenylbutyrate (phenylbutyrate) (Engelhard et al. (1998) J. NEUROONCOL. 37(2): 97-108), Compound 0012 (Hurley et al. (2010) J. VASC. RES. 47(1): 80-90), curcumin (Aggarwal et al. (2005) CLIN. CANCER RES. 11(20): 7490-8), magnesium hydroxide (Mori et al. (1997) J. CELL BIOCHEM. SUPPL. 27:35-41), BP-1-102 (Zhang et al. (2012) PROC. NATL. ACAD.SCI. U.S.A. 109(24):9623-8), WP1193 (Sai et al. (2012) J. NEUROONCOL. 107(3): 487-501), BP-1-107 (Page et al. (2012) J. MED. CHEM. 55(3):1047-55), BP-1-108 (Page et al. (2012) J. MED. CHEM. 55(3): 1047-55), SF-1-087 (Page et al. (2012) J. MED. CHEM. 55(3):1047-55), SF-1-088 (Page et al. (2012) J. MED. CHEM. 55(3):1047-55), STX-0119 (Ashizawa et al. (2011) INT. J. ONCOL. 38(5):1245-52), substituted thiazol-4-one compounds (U.S. Pat. No. 7,872,027), (Z,E)-5-(4-ethylbenzylidene)-2-thioxothiazolidin-4-one (10058-F4) (U.S. Pat. No. 7,026,343), S2T1-60TD (U.S. Publication No. 20120107317A1), Quarfloxin (CX-3543) (U.S. Publication No. 20120107317A1), benzoylanthranilic acid (U.S. Publication No. 20120107317A1), cationic porphyrin TMPyP4 (U.S. Publication No. 20120107317A1), tyrphostin and tryphostin-like compounds (European Patent No. EP2487156A1), AG490 (European Patent No. EP2487156A1), FBXW-7 expression vectors (Ishikawa Y et al., supra), and siRNAs targeting c-Myc transcript (Id.).

Exemplary Notch activators include microRNAs that target FBXW-7 (Ishikawa Y et al. supra), AG-370, 5 (U.S. Pat. No. 8,114,422), AG-1296 (6,7-dimethoxy-3-phenylquinoxaline) (Id.), nigericin·Na (Id.), cytochalasin D (Id.), FCCP (carbonylcyanide-4-(trifluoromethoxy)-phenylhydrazone) (Id.), SP60012 (Id.), and vectors that produce protein of or isolated protein of Jagged-1, Jagged-2, Jagged-3, Serrate, any member of the Jagged/Serrate protein family, Delta, Delta-like-1, Delta-like-3, Delta-like-4, Delta-like homolog-1 (DLK1), any member of the Delta protein family, and any portion of any of these proteins (PCT Publication WO2004090110A3). Exemplary Notch activators may also include chemical activators such as valproic acid (VPA, see, U.S. Pat. No. 8,338,482), resveratrol and phenethyl isothiocyanate.

Exemplary Notch inhibitors include gamma-secretase inhibitors such as an arylsulfonamide, a benzodiazepine, L-685,458 (U.S. Patent Publication No. 2001/0305674), MK-0752 (Purow B. (2012) ADV. EXP. MED. BIOL. 727:305-19; Imbimbo B P (2008) CURR. TOP. MED. CHEM. 8(1):54-61), DAPT ([N-(3,5-Difluorophenacetyl)-L-alanyl]-S-phenyl glycine t-butyl ester) (Id.; Ishikawa Y et al. supra; PCT Publication WO2011149762A3), LY-374973 (N-[N-(3,5-Difluorophenacctyl)-L-alanyl]-S-phenylglycine t-butyl ester) (PCT Publication WO2011149762A3), N-[(3,5-Difluorophenyl)acetyl]-L-alanyl-2-phenyl]glycine-1,1-dimethylethyl ester (Id.); Lilly GSI L685,458 (Purow B, supra), compound E ((2S)-2-{[(3,5-Difluorophenyl)acetyl]amino}-N-[(3S)-1-methyl-2-oxo-5-phenyl-2,3-dihydro-1H-1,4-benzodiazepin-3-yl]propanamide) (Purow B, supra), DBZ (dibenzazepine) (Purow B, supra), isocoumarin (Purow B, supra), JLK6(7-amino-4-chloro-3-methoxyisocoumarin) (Purow B (2012) ADV. EXP. MED. BIOL. 727:305-19), Compound 18 ([11-endo]-N-(5,6,7,8,9,10-hexahydro-6,9-methano benzo[9][8]annulen-11-yl)-thiophene-2-sulfonamide) (Purow B, supra), E2012 (Imbimbo B P, supra; PCT Publication WO2009005688A3), MRK560 (Imbimbo B P, supra), LY-411575 (Imbimbo B P, supra), LY-450139 (Imbimbo B P, supra; PCT Publication WO2009005688A3), γ-secretase inhibitor XII (PCT Publication WO2011149762A3; PCT Publication WO2009005688A3), 2, 2-dimethyl-N-((S)-6-oxo-6, 7-dihydro-5H-dibenzo(b, d)azepin-7-yl)-N′-(2, 2, 3, 3, 3-pentafluoro-propyl)-malonamide (U.S. Patent Publication No. 20090181944A1), GSI-IX (EP1949916B1), GSI-X (EP1949916B1), tocopherol derivatives (PCT Publication WO2009040423A1), [(2S)-2-{[(3,5-Difluorophenyl)acetyl]aminoτ-N-[(3S)1-methyl-2-oxo-5-phenyl-2,3-dihydro-1 H-1,4-benzodiazepin-3-yl] propanamide] (PCT Publication WO2009005688A3), N-[N-(3,5-difluorophenacetyl)-L-alanyl]-Sphenylglycine-t-butylester (Id.), [1,1′-Biphenyl]-4-acetic acid (Id.), 2-fluoro-alpha-methyl (Id.), NGX-555 (Id.), LY-411575 (Id.), Cellzome (Id.), 2-Thiophenesulfonamide (Id.), 5-chloro-N-[(1S)-3,3,3-trifluoro-1-(hydroxymethyl)-2-(trifluoromethyl)propyl] (Id.), NIC5-15 (Id.), BMS (Id.), CHF-5074 (Id.), BMS-299897 (Imbimbo B P, supra), RO4929097; L-685458 ((5S)-(t-Butoxycarbonylamino)-6-phenyl-(4R)hydroxy-(2R)benzylhexanoyl)-L-leu-L-phe-amide); BMS-708163 (Avagacestat); BMS-299897 (2-[(1R)-1-[[(4-Chlorophenyl)sulfonyl](2,5-difluorophenyl)amino]ethyl-5-fluorobenzenebutanoic acid); MK-0752; YO-01027; MDL28170 (Sigma); LY411575 (N-2((2S)-2-(3,5-difluorophenyl)-2-hydroxyethanoyl)-N1-((7S)-5-methyl-6-oxo-6,7-dihydro-5H-dibenzo [b,d]azepin-7-yl)-1-alaninamide, sec U.S. Pat. No. 6,541,466); ELN-46719 (2-hydroxy-valeric acid amide analog of LY411575 (where LY411575 is the 3,5-difluoro-mandelic acid amide) (U.S. Pat. No. 6,541,466)); PF-03084014 ((S)-2-((S)-5,7-difluoro-1,2,3,4-tetrahydronaphthalen-3-ylamino)-N-(1-(2-methyl-1-(ncopentylamino) propan-2-yl)-1H-imidazol-4-yl) pentanamide, Samon et al., MOLCANCER THER 2012;11:1565-1575); and Compound E ((2S)-2-{[(3,5-Diflurophenyl)acetyl]amino}-N-[(3S)-1-methyl-2-oxo-5-phenyl-2,3-dihydro-1H-1,4-benzodiazepin-3-yl]propanamide; sec WO 98/28268 and Samon et al., MOL CANCER THER 2012; 11:1565-1575; available from Alexis Biochemicals)), or pharmaceutically acceptable salts thereof. In some embodiments, suitable gamma secretase inhibitors include: semagacestat (also known as LY450139, (2S)-2-hydroxy-3-methyl-N-[(1S)-1-methyl-2-oxo-2-[[(1S)-2,3,4,5-tetrahydro-3-methyl-2-oxo-1H-3-benzazepin-1-yl]amino]ethyl]butanamide, available from Eli Lilly; WO 02/47671 and U.S. Pat. No. 7,468,365); LY411575 (N-2((2S)-2-(3,5-difluorophenyl)-2-hydroxyethanoyl)-N1-((7S)-5-methyl-6-oxo-6,7-dihydro-5H-dibenzo[b,d]azepin-7-yl)-L-alaninamide, available from Eli Lilly, Fauq et al., (2007) BIOORG MED CHEM LETT 17:6392-5);begacestat (also known as GSI-953, U.S. Pat. No. 7,300,951);arylsulfonamides (A S, Fuwa et al., (2006) BIOORG MED CHEM LETT. 16(16):4184-4189); N-[N-(3,5-difluorophenacetyl)-L-alanyl]-(S)-phenylglycine L-butyl ester (DAPT, Shih et al., (2007) CANCER RES. 67:1879-1882); N-[N-3,5-Difluorophenacetyl]-L-alanyl-S-phenylglycine Methyl Ester (also known as DAPM, gamma-Secretase Inhibitor XVI, available from EMD Millipore); Compound W (3,5-bis(4-Nitrophenoxy)benzoic acid, available from Tocris Bioscience); L-685,458 ((5S)-(tert-Butoxycarbonylamino)-6-phenyl-(4R)-hydroxy-(2R)-benzylhexanoyl)-L-leucy-L-phenylalaninamide, available from Sigma-Aldrich, Shearmen et al., (2000) BIOCHEMISTRY 39, 8698-8704); BMS-289948 (4-chloro-N-(2,5-difluorophenyl)-N-((1R)-{4-fluoro-2-[3-(1H-imidazol-1-yl)propyl]phenyl}ethyl)benzenesulfonamide hydrochloride, available from Bristol Myers Squibb); BMS-299897 (4-[2-((1R)-1-{[(4-chlorophenyl)sulfonyl]-2,5-difluoroanilino}ethyl)-5-fluorophenyl]butanoic acid, available from Bristol Myers Squibb, sec Zheng et al., (2009) XENOBIOTICA 39(7):544-55); avagacestat (also known as BMS-708163, (R)-2-(4-chloro-N-(2-fluoro-4-(1,2,4-oxadiazol-3-yl) benzyl) phenylsulfonamido)-5,5,5-trifluoropentanamide, available from Bristol Myers Squibb, Albright et al., (2013) J PHARMACOL. EXP. THER. 344(3):686-695); MK-0752 (3-(4-((4-chlorophenyl)sulfonyl)-4-(2,5-difluorophenyl)cyclohexyl)propanoic acid, available from Merck); MRK-003 ((3′R,6R,9R)-5′-(2,2,2-trifluorocthyl)-2-((E)-3-(4-(trifluoromethyl) piperidin-1-yl) prop-1-cn-1-yl)-5,6,7,8,9,10-hexahydrospiro[6,9-methanobenzo [8]annulene-11,3′-[1,2,5]thiadiazolidine]1′,1′-dioxide, available from Merck, Mizuma et al., (2012) MOL CANCER THER. 11(9):1999-2009); MRK-560(N-[cis-4-[(4-Chlorophenyl) sulfonyl]-4-(2,5-difluorophenyl) cyclohexyl]-1,1,1-trifluoro-methanesulfonamide, Best et. al., (2006) J PHARMACOL EXP Ther. 317(2):786-90); RO-4929097 (also known as R4733, (S)-2,2-dimethyl-N1-(6-oxo-6,7-dihydro-5H-dibenzo[b,d]azepin-7-yl)-N3-(2,2,3,3,3-pentafluoropropyl)malonamide, available from Hoffman-La Roche Inc., Tolcher et al., (2012) J CLIN. ONCOL. 30(19):2348-2353); JLK6 (also known as 7-Amino-4-chloro-3-methoxyisocoumarin, available from Santa Cruz Biotechnology, Inc., Petit et al., (2001) NAT. CELL. BIOL. 3:507-511); Tarenflurbil (also known as (R)-Flurbiprofen, (2R)-2-(3-fluoro-4-phenylphenyl)propanoic acid); ALX-260-127 (also known as Compound 11, described by Wolfe et al. (1998) J. MED. CHEM. 41:6); Sulindac sulfide (SSide, et al., (2003) J BIOL . CHEM. 278(20): 18664-70); 1,1,1-trifluoro-N-(4-[5-fluoro-2-(trifluoromethyl)phenyl]-4-{[4(trifluoromethyl)phenyl]sulfonyl}cyclohexyl)methanesulfonamide (U.S. Patent Publication No. 20110275719);N-[trans-3-[(4-chlorophenyl)sulfonyl]-3-(2,5-difluorophenyl)cyclobutyl]-1,1,1-trifluoromethanesulfonamide (U.S. Patent Publication No. 20110263580);N-[cis-3-[(4-chlorophenyl)sulfonyl]-3-(2,5-difluorophenyl) cyclobutyl]-1,1,1-trifluoromethanesulfonamide (U.S. Patent Publication No. 20110263580); N-[cis-3-[(4-chlorophenyl) sulfonyl]-3-(2-cyano-5-fluorophenyl)cyclobutyl]-1,1,1-trifluoromethanesulfonamide (U.S. Patent Publication No. 20110263580);N-[cis-3-[(4-chlorophenyl)sulfonyl]-3-(2,5-dichlorophenyl)cyclobutyl]-1,1,1-trifluoromethanesulfonamide (U.S. Patent Publication No. 20110263580); N-(cis-3-(2,5-difluorophenyl)-3-{[4-(trifluoromethyl)phenyl]sulfonyl}cyclobutyl)-1,1,1-trifluoromethanesulfonarnide (U.S. Patent Publication No. 20110263580); N-{cis-3-(5-chloro-2-fluorophenyl)-3-[(4-chlorophenyl)sulfonyl}cyclobutyl569 -1,1,1-trifluoromethanesulfonamide (U.S. Patent Publication No. 20110263580);N-{cis-3-(2,5-difluorophenyl)-3-[(4-fluorophenyl)sulfonyl]cyclobutyl]-1,1,1-trifluoromethanesulfonamide (U.S. Patent Publication No. 20110263580);N-{cis-3-(2,5-difluorophenyl)-3-[(3,4-difluorophenyl)sulfonyl]cyclobutyl}-1,1,1-trifluoromethanesulfonamide (U.S. Patent Publication No. 20110263580); N-[cis-3-[(4-cyanophenyl)sulfonyl]-3-(2,5-difluorophenyl)cyclobutyl]-1,1,1-trifluoromethanesulfonamide (U.S. Patent Publication No. 20110263580);N-[cis-3-[(3,4-chlorophenyl)sulfonyl]-3-(2,5-difluorophenyl)cyclobutyl][trifluoromethyl) sulfonyl]amino}butanoic acid (U.S. Patent Publication No. 20110263580);N-[cis-3-[(4-chlorophenyl)sulfonyl]-3-(2,5-difluorophenyl)cyclobutyl]-1,1,1-trifluoro-N-[2-(tetrahydro-2-pyran-2-yloxy)ethyl]methanesulfonamide (U.S. Patent Publication No. 20110263580); Methyl{[cis-3-[(4-chlorophenyl)sulfonyl]-3-(2,5-difluorophenyl)cyclobutyl][(trifluoromethyl)sulfonyl]amino}acetate (U.S. Patent Publication No. 20110263580);N-[3-[(4-chlorophenyl)sulfonyl]-3-(2,5-difluorophenyl)cyclobutyl]-1,1,1-trifluoro-N-methylmethanesulfonamide (U.S. Patent Publication No. 20110263580); N-[3-[(4-chlorophenyl)sulfonyl]-3-(2,5-difluorophenyl) cyclobutyl]-1,1,1-trifluoro-N-methylmethanesulfonamide (U.S. Patent Publication No. 20110263580); Methyl 4-{[cis-3-[(4-chlorophenyl)sulfonyl]-3-(2,5-difluorophenyl)cyclobutyl] [(trifluoromethyl)sulfonyl]amino}butanoate (U.S. Patent Publication No. 20110263580); N-[cis-3-[(4-chlorophenyl)sulfonyl]-3-(2,5-difluorophenyl) cyclobutyl]-N-[(trifluoromethyl)sulfonyl]glycine (U.S. Patent Publication No. 20110263580);N-[cis-3-[(4-chlorophenyl)sulfonyl]-3-(2,5-difluorophenyl)-1-methylcyclobutyl]-1,1,1-trifluoromethanesulfonamide (U.S. Patent Publication No. 20110263580); N-(cis-3-(2,5-difluorophenyl)-1-methyl-3-{[4-(trifluoromethyl)phenyl]sulfonyl}cyclobutyl)-1,1,1-trifluoromethanesulfonamide (U.S. Patent Publication No. 20110263580);N-[cis-3-[(4-chlorophenyl)sulfonyl]-3-(2,5-difluorophenyl)cyclobutyl]-1,1,1-trifluoro-N-[(trifluoromethyl)sulfonyl]methanesulfonamide (U.S. Patent Publication No. 20110263580); Sodium[cis-3-[(4-chlorophenyl)sulfonyl]-3-(2,5-difluorophenyl)cyclobutyl][(trifluoromethyl)sulfonyl]Jzanide (U.S. Patent Publication No. 20110263580);Potassium [cis-3-[(4-chlorophenyl)sulfonyl]-3-(2,5-difluorophenyl)cyclo butyl][(trifluoromethyl)sulfonyl]azanide (U.S. Patent Publication No.20110263580); N-[cis-3-[(4-trifluoromethoxyphenyl)sulfonyl]-3-(2,5-difluorophenyl)cyclobutyl]-1,1,1-trifluoromethanesulfonamide (U.S. Patent Publication No. 20110263580);1,1,1-trifluoro-N-(4-[5-fluoro-2-(trifluoromethyl)phenyl]-4-{[4-(trifluoromethyl)phenyl]sulfonyl}cyclohexyl)methanesulfonamide (U.S. Patent Publication No. 20110263580); gamma-Secretase Inhibitor I (also known as Z-Leu-Leu-Nle-CHO, benzyloxycarbonyl-leucyl-leucyl-norleucinal, available from Calbiochem); gamma-secretase inhibitor II:

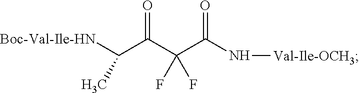

(MOL)(CDX) (available from Calbiochem);gamma secretase inhibitor III, (N-Benzyloxycarbonyl-Leu-leucinal, available from Calbiochem);gamma secretase inhibitor IV, (N-(2-Naphthoyl)-Val-phenylalaninal, available from Calbiochem);gamma-secretase inhibitor V (also known as Z-LF-CHO, N-Benzyloxycarbonyl-Leu-phenylalaninal, available from EMD Millipore);gamma-secretase inhibitor VI (1-(S)-endo-N-(1,3,3)Trimethylbicyclo[2.2.1]hept-2-yl)-4-fluorophenyl Sulfonamide, available from EMD Millipore);gamma secretase inhibitor VII, (also known as Compound A, MOC-LL-CHO, Menthyloxycarbonyl-LL-CHO, available from Calbiochem);gamma secretase inhibitor X, ({1S-Benzyl-4R-[1-(1S-carbamoyl-2-phenethylcarbamoyl)-1S-3-methylbutylcarbamoyl]-2R- hydroxy-5-phenylpentyl}carbamic acid tert-butyl ester, available from Calbiochem); gamma secretase inhibitor XI, (7-Amino-4-chloro-3-methoxyisocoumarin, available from Calbiochem);gamma secretase inhibitor XII, (also known as Z-Ile-Leu-CHO, Shih and Wang, (2007) CANCER RES. 67:1879-1882);gamma secretase inhibitor XIII, (Z-Tyr-Ile-Leu-CHO, available from Calbiochem);gamma secretase inhibitor XIV, (Z-Cys(t-Bu)-Ile-Leu-CHO, available from Calbiochem);gamma secretase inhibitor XVII, (also known as WPE-III-31C),

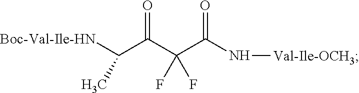

(MOL)(CDX) (available from Calbiochem);gamma secretase inhibitor XIX, (also known as benzodiazepine, (2S,3R)-3-(3,4-Difluorophenyl)-2-(4-fluorophenyl)-4-hydroxy-N-((3S)-2-oxo-5-phenyl-2,3-dihydro-1H-benzo[e][1,4]diazepin-3-yl)-butyramide, Churcher et al., (2003) J MED CHEM. 46(12):2275-8);gamma secretase inhibitor XX, (also known as dibenzazepine, (S,S)-2-[2-(3,5-Difluorophenyl)acetylamino]-N-(5-methyl-6-oxo-6,7-dihydro-5H-dibenzo[b,d]azepin-7-yl)propionamide,

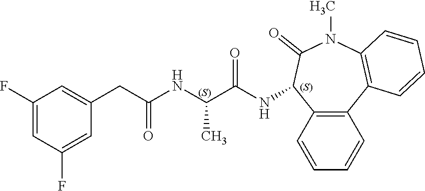

(MOL)(CDX) (Weihofen et al., Science 296:2215-2218, 2002, available from Calbiochem);gamma secretase inhibitor XXI, ((S,S)-2-[2-(3,5-Difluorophenyl)-acetylamino]-N-(1-methyl-2-oxo-5-phenyl-2,3-dihydro-1H-benzo[e][1,4]diazepin-3-yl)-propionamide, available from Calbiochem);5-methyl-2-propan-2-ylcyclohexyl)N-[4-methyl-1-[(4-methyl-1-oxopentan-2-yl)amino]-1-oxopentan-2-yl]carbamate (available from HDH Pharma Inc.);N-trans-3,5-Dimethoxycinnamoyl-Ile-leucinal (available from Calbiochem);N-tert-Butyloxycarbonyl-Gly-Val-Valinal; isovaleryl-V V-Sta-A-Sta-OCH3 (available from Calbiochem);diethyl-(5-phenyl-3H-azepin-2-yl)-amine (U.S. Pat. No. 8,188,069);diethyl-(5-isopropyl-3H-azepin-2-yl)-amine (U.S. Pat. No. 8,188,069); diethyl-(4-phenyl-3H-azepin-2-yl)-amine (U.S. Pat. No. 8,188,069); diethyl-(6-phenyl-3H-azepin-2-yl)-amine (U.S. Pat. No. 8,188,069);5-phenyl-1,3-dihydro-azepin-2-one (U.S. Pat. No. 8,188,069);5-Isopropyl-1,3-dihydro-azepin-2-one (U.S. Pat. No. 8,188,069);4-phenyl-1,3-dihydro-azepin-2-one (U.S. Pat. No. 8,188,069);6-phenyl-1,3-dihydro-azepin-2-one (U.S. Pat. No. 8,188,069);2-butoxy-5-phenyl-3H-azepine (U.S. Pat. No. 8,188,069);1-methyl-5-phenyl-1,3-dihydro-azepin-2-one (U.S. Pat. No. 8,188,069);5-isopropyl-1-methyl-1,3-dihydro-azepin-2-one (U.S. Pat. No. 8,188,069); 1-methyl-4-phenyl-1,3-dihydro-azepin-2-one (U.S. Pat. No. 8,188,069); 1-methyl-6-phenyl-1,3-dihydro-azepin-2-one (U.S. Pat. No. 8,188,069); 1-methyl-5-phenyl-1H-azepine-2,3-dione-3-oxime (U.S. Pat. No. 8,188,069);5-isopropyl-1-methyl-1H-azepine-2,3-dione-3-oxime (U.S. Pat. No. 8,188,069);1-methyl-6-phenyl-1H-azepine-2,3-dione-3-oxime (U.S. Pat. No. 8,188,069); 1-methyl-4-phenyl-1H-azepine-2,3-dione-3-oxime (U.S. Pat. No. 8,188,069);3-amino-1-methyl-5-phenyl-1,3-dihydro-azepin-2-one (U.S. Pat. No. 8,188,069);3-amino-5-isopropyl-1-methyl-1,3-dihydro-azepin-2-one (U.S. Pat. No. 8,188,069);3-amino-1-methyl-4-phenyl-1,3-dihydro-azepin-2-one (U.S. Pat. No. 8,188,069);3-amino-1-methyl-6-phenyl-1,3-dihydro-azepin-2-one (U.S. Pat. No. 8,188,069);(S)-[1-(1-methyl-2-oxo-5-phenyl-2,3-dihydro-1H-azepin-3-ylcarbamoyl)-ethyl]-carbamic acid tertbutyl ester (U.S. Pat. No. 8,188,069);[(S)-1-(5-isopropyl-1-methyl-2-oxo-2,3-dihydro-1H-azepin-3-ylcarbamoyl)-ethyl]carbamic acid tert-butyl ester (U.S. Pat. No. 8,188,069);[(S)-1-(1-methyl-2-oxo-4-phenyl-2,3-dihydro-1H-azepin-3-ylcarbamoyl)-ethyl]carbamic acid tert-butyl ester (U.S. Pat. No. 8,188,069);[(S)-1-(1-methyl-2-oxo-6-phenyl-2,3-dihydro-1H-azepin-3-ylcarbamoyl)-ethyl]-carbamic acid tert-butyl ester (U.S. Pat. No. 8,188,069);(S)-2-amino-N-(1-methyl-2-oxo-5-phenyl-2,3-dihydro-1H-azepin-3-yl)-propionamide (U.S. Pat. No. 8,188,069);(S)-2-amino-N-(5-isopropyl-1-methyl-2-oxo-2,3-dihydro-1H-azepin-3-yl) propionarnide (U.S. Pat. No. 8,188,069);(S)-2-Amino-N-(1-methyl-2-oxo-6-phenyl-2,3-dihydro-1H-azepin-3-yl)propionamide hydrochloride (U.S. Pat. No. 8,188,069);(S)-2-Amino-N-(1-methyl-2-oxo-4-phenyl-2,3-dihydro-1H-azepin-3-yl) propionamide hydrochloride (U.S. Pat. No. 8,188,069);(S)-2-fluoro-3-methyl-butyric acid (U.S. Pat. No. 8,188,069);(S)-2-hydroxy-3-methyl-N-[(S)-1-((S)-1-methyl-2-oxo-5-phenyl-2,3-dihydro-1H-azepin-3-ylcarbamoyl)-ethyl]-butyramide (U.S. Pat. No. 8,188,069);(S)-2-fluoro-3-methyl-N-[(S)-1-(1-methyl-2-oxo-5-phenyl-2,3-dihydro-1H-azepin-3-ylcarbamoyl)-ethyl]-butyramide (U.S. Pat. No. 8,188,069);(S)-2-hydroxy-N-[(S)-1-(5-isopropyl-1-methyl-2-oxo-2,3-dihydro-1H-azepin-3-ylcarbamoyl)-ethyl]-3-methyl-butyramide (U.S. Pat. No. 8,188,069);(S)-2-hydroxy-3-methyl-N-[(S)-1-(1-methyl-2-oxo-4-phenyl-2,3-dihydro1H-azepin-3-ylcarbamoyl)-ethyl]-butyramide (U.S. Pat. No. 8,188,069);(S)-2-hydroxy-3-methyl-N-[(S)-1-(1-methyl-2-oxo-6-phenyl-2,3-dihydro-1H-azepin-3-ylcarbamoyl)-ethyl]-butyramide (U.S. Pat. No. 8,188,069); and (S)-2-fluoro-3-methyl-N-[(S)-1-(1-methyl-2-oxo-6-phenyl-2,3-dihydro-IH-azepin-3ylcarbamoyl)-cthyl]-butyramide (U.S. Pat. No. 8,188,069), and pharmaceutically acceptable salts thereof.

Additional examples of gamma-secretase inhibitors are disclosed in U.S. Patent Application Publication Nos. 2004/0029862, 2004/0049038, 2004/0186147, 2005/0215602, 2005/0182111, 2005/0182109, 2005/0143369, 2005/0119293, 2007/0190046, 2008/008316, 2010/0197660 and 2011/0020232; U.S. Pat. Nos. 6,756,511; 6,890,956; 6,984,626; 7,049,296; 7,101,895; 7,138,400; 7,144,910; 7,183,303; 8,188,069; and International Publication Nos. WO 1998/28268; WO 2001/70677, WO 2002/049038, WO 2004/186147, WO 2003/093253, WO 2003/093251, WO 2003/093252, WO 2003/093264, WO 2005/030731, WO 2005/014553, WO 2004/039800, WO 2004/039370, WO 2009/023453, EP 1720909, EP 2178844, EP 2244713.

Additional exemplary Notch inhibitors include nonsteroidal anti-inflammatory drugs (NSAIDs) such as flurbiprofen (Purow B, supra), MPC-7869 (Imbimbo B P, supra), ibuprofen (Id.), sulindac sulphide, indomethacin, alpha-secretase inhibitors (ASIs) (Purow B, supra), the Na+/H+ antiporter Monensin (Id.); small molecules that block Notch binding to interacting proteins such as Jagged, Numb, Numb-like, CBF1 transcription factor, and mastermind-like (MAML) (Id.; Ishikawa Y et al, supra.); antibodies that bind Notch proteins or Notch ligands such as Delta-Like-4 (Purow B, supra); stapled peptides that bind Notch such as SAHMI (Id.); dominant-negative forms of genes such as MAML (Id; Ishikawa Y et al., supra), Numb/Numb-Like (Purow B, supra), and FBXW-7 (Id.); expression vectors that increase levels of Notch regulators such as FBXW-7 (Id.; Ishikawa Y et al., supra); siRNAs that target Notch transcripts (Purow B, supra); microRNAs such as miR-326, miR-34a, microRNA-206, and miR-124 (Id.); and Notch antibodies (U.S. Pat. No. 8,226,943, U.S. Publication No. 20090258026A2, PCT Publication WO2012080926A2).

Exemplary Atoh1 activators include, for example, β-Catenin or β-catenin pathway agonists, e.g., Wnt ligands, DSH/DVL1, 2, 3, LRP6δN, WNT3A, WNT5A, and WNT3A, 5A. Additional Wnt/β-catenin pathway activators and inhibitors are reviewed in the art (Moon et al., Nature Reviews Genetics, 5:689-699, 2004). In some embodiments, suitable Wnt/β-catenin pathway agonists can include antibodies and antigen binding fragments thereof, and peptides that bind specifically to frizzled (Fzd) family of receptors.

Kinase inhibitors, e.g., casein kinase 1 (CK1) and glycogen synthase kinase 3β (GSK3β) inhibitors can also act as β-Catenin or βcatenin pathway agonists to activate Atoh1. GSK3β inhibitors include, but are not limited to, lithium chloride (LiCl), Purvalanol A, olomoucine, alsterpaullone, kenpaullone, benzyl-2-methyl-1,2,4-thiadiazolidine-3,5-dione (TDZD-8), 2-thio (3-iodobenzyl)-5-(1-pyridyl)-[1,3,4]-oxadiazole (GSK3 inhibitor II), 2,4-dibenzyl-5-oxothiadiazolidine-3-thione (OTDZT), (2′Z,3′E)-6-Bromoindirubin-3′-oxime (BIO), α-4-Dibromoacetophenone (i.e., Tau Protein Kinase I (TPK I) Inhibitor), 2-Chloro-1-(4,5-dibromo-thiophen-2-yl)-ethanone N-(4-Methoxybenzyl)-N′-(5-nitro-1,3-thiazol-2-yl) urea (AR-A014418), and indirubins (e.g., indirubin-5-sulfonamide; indirubin-5-sulfonic acid (2-hydroxyethyl)-amide indirubin-3′-monoxime; 5-iodo-indirubin-3′-monoxime; 5-fluoroindirubin; 5,5′-dibromoindirubin; 5-nitroindirubin; 5-chloroindirubin; 5-methylindirubin, 5-bromoindirubin), 4-Benzyl-2-methyl-1,2,4-thiadiazolidine-3,5-dione (TDZD-8), 2-thio (3-iodobenzyl)-5-(1-pyridyl)-[1,3,4]-oxadiazole (GSK3 inhibitor II), 2,4-Dibenzyl-5-oxothiadiazolidine-3-thione (OTDZT), (2′Z,3′E)-6-Bromoindirubin-3′-oxime (BIO), α-4-Dibromoacetophenone (i.e., Tau Protein Kinase I (TPK I) Inhibitor), 2-Chloro-1-(4,5-dibromo-thiophen-2-yl)-ethanone, (vi) N-(4-Methoxybenzyl)-N′-(5-nitro-1,3-thiazol-2-yl) urea (AR-A014418), and H-KEAPPAPPQSpP-NH2 (L803) or its cell-permeable derivative Myr-N-GKEAPPAPPQSpP-NH2 (L803-mts). Other GSK3β inhibitors are disclosed in U.S. Pat. Nos. 6,417,185; 6,489,344; and 6,608,063. In some embodiments, suitable kinase inhibitors can include RNAi and siRNA designed to decrease GSK3β and/or CK1 protein levels. In some embodiments, useful kinase inhibitors include FGF pathway inhibitors. In some embodiments, FGF pathway inhibitors include, for example, SU5402.

Additional Atoh1 activators include gamma secretase inhibitors (e.g., arylsulfonamides, dibenzazepines, benzodiazepines, N-[N-(3,5-difluorophenacetyl)-L-alanyl]-(S)-phenylglycine t-butyl ester (DAPT; EMD Biosciences, San Diego, CA, USA), L-685,458, or MK0752ho, in addition to those listed above under Notch inhibitors), gentamycin, and the combination of transcription factors Eya1 and Six1 (and optionally Sox2), as described in Ahmed et al. (2012) DEV. CELL 22(2):377-390.

Additional Atoh1 activators are described in U.S. Pat. No. 8,188,131, including a compound represented by Formula I:

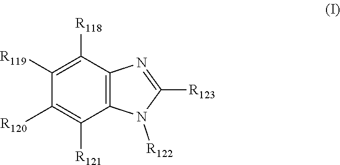

wherein:
each of R₁₁₈, R₁₁₉, R₁₂₀, and R₁₂₁ is, independently selected from H, halo, OH, CN, NO₂, C₁-C₃ alkyl, C₁-C₃ haloalkyl, C₁-C₃ alkoxy, and C₁-C₃ haloalkoxy;
R₁₂₂ is hydrogen or —Z—R^{a}; wherein:
Z is O or a bond; and
R^{a} is:
   (i) C₁-C₆ alkyl or C₁-C₆ haloalkyl, each of which is optionally substituted with from 1-5 R^{b}; or
   (ii) C₃-C₁₀ cycloalkyl, C₃-C₁₀ cycloalkenyl, each of which is optionally substituted with from 1-5 R^{c}; or
   (iii) C₇-C₁₁ aralkyl, or heteroaralkyl including 6-11 atoms, each of which is optionally substituted with from 1-5 R^{c};
(iv) C₆-C₁₀ aryl or heteroaryl including 5-10 atoms, each of which is optionally substituted with from 1-5 R_{d};
R₁₂₃ is:
   (i) hydrogen; or
   (ii) C₁-C₆ alkyl or C₁-C₆ haloalkyl, each of which is optionally substituted with from 1-3 R^{b}; or
   (iii) C₆-C₁₀ aryl or heteroaryl including 5-10 atoms, each of which is optionally substituted with from 1-5 R^{d}; or
   (iv) C₇-C₁₁ aralkyl, or heteroaralkyl including 6-11 atoms, each of which is optionally substituted with from 1-5 R^{c}; or
   (v)-(C₁-C6 alkyl)-Z¹-(C₆-C₁₀ aryl), wherein Z¹ is O, S, NH, or N (CH₃); the alkyl portion is optionally substituted with from 1-3 R^{b}; and the aryl portion is optionally substituted with from 1-5 R^{d}; or
   (vi)-(C₁-C₆ alkyl)-Z²-(heteroaryl including 5-10 atoms), wherein Z² is O, S, NH, or N (CH₃); the alkyl portion is optionally substituted with from 1-3 R^{b}; and the heteroaryl portion is optionally substituted with from 1-5 R^{d}; or
   (vii)-(C₁-C₆ alkyl)-Z³-(C₃-C₁₀ cycloalkyl), wherein Z³ is O, S, NH, or N (CH₃); the alkyl portion is optionally substituted with from 1-3 R^{b}; and the cycloalkyl portion is optionally substituted with from 1-5 R^{c};
R^{b} at each occurrence is, independently:
   (i) NH₂; NH(C₁-C₃ alkyl); N(C₁-C₃ alkyl)₂; hydroxy; C₁-C₆ alkoxy or C₁-C₆ haloalkoxy; or
   (ii) C₃-C₇ cycloalkyl optionally substituted with from 1-3 substituents independently selected from C₁-C₆ alkyl, NH₂; NH(C₁-C₃ alkyl); N(C₁-C₃ alkyl)₂; hydroxy; C₁-C₆ alkoxy or C₁-C₆ haloalkoxy;
R^{c} at each occurrence is, independently:
   (i) halo; NH₂; NH(C₁-C₃ alkyl); N(C₁-C₃ alkyl)₂; hydroxy; C₁-C₆ alkoxy; C₁-C⁶ haloalkoxy; or oxo; or
   (ii) C₁-C₆ alkyl or C₁-C₆ haloalkyl; and
R^{d} at each occurrence is, independently:
   (i) halo; NH₂; NH(C₁-C₃ alkyl); N(C₁-C₃ alkyl)₂; hydroxy; C₁-C₆ alkoxy or C₁-C₆ haloalkoxy; nitro; —NHC(O)(C₁-C₃alkyl); or cyano; or
   (ii) C₁-C6 alkyl or C₁-C₆ haloalkyl; or a pharmaceutically acceptable salt thereof.

Other exemplary Atoh1 activators described in U.S. Pat. No. 8,188,131 include 4-(4-chlorophenyl)-1-(5H-pyrimido[5,4-b]indol-4-yl)-1H-pyrazol-3-amine; 6-chloro-1-(2-chlorobenzyloxy)-2-phenyl-1H-benzo[d]imidazole; 6-chloro-1-(2-chlorobenzyloxy)-2-(4-methoxyphenyl)-1H-benzo[d]imidazole; 6-chloro-2-(4-methoxyphenyl)-1-(4-methylbenzyloxy)-1H-benzo[d]imidazole; 6-chloro-1-(3,5-dimethylbenzyloxy)-2-(4-methoxyphenyl)-1H-benzo[d]imidazole; 6-chloro-1-(4-methoxybenzyloxy)-2-(4-methoxyphenyl)-1H-benzo[d] imidazole; 1-(4-methylbenzyloxy)-6-nitro-2-phenyl-1H-benzo [d]imidazole; 4-(1H-benzo[d] imidazol-2-yl) phenol; 2,5-dichloro-N-((1-methyl-1H-benzo[d]imidazol-2-yl) methyl) aniline; 4-(2-(1-methyl-1H-benzo[d]imidazol-2-yl)ethyl)aniline; 2-((2-methoxyphenoxy) methyl)-1H-benzo[d]imidazole; 2-((4-fluorophenoxy)methyl)-1-methyl-1H-benzo[d]imidazole; 2-(phenylthiomethyl)-1H-benzo[d]imidazole; 3-(6-methyl-1H-benzo[d]imidazole-2-yl)-2H-chromen-2-iminc; N-(2-(1H-benzo [d]imidazole-2-yl)phenyl)isobutyramide; 2-(o-tolyloxymethyl)-1H-benzo [d]imidazole; 2-(4-methoxyphenyl)-1-phenethyl-1H-benzo[d]imidazole; N-(6-bromobenzo[d]thiazole-2-yl) thiophene-2-carboxamide; N-(benzo[d]thiazole-2-yl)-1-methyl-1H-pyrazole-5-carboxamide; 2-(4-fluorobenzylthio)benzo[d]thiazole; 5-chloro-N-methylbenzo[d]thiazole-2-amine; N-(6-acetamidobenzo[d]thiazol-2-yl)furan-2-carboxamide; N-(6-fluorobenzo[d]thiazole-2-yl)-3-methoxybenzamide; 2-(benzo[d]oxazol-2-ylthio)-N-(2-chlorophenyl)acetamide; 5-chloro-2-phenylbenzo[d]oxazole; 5-methyl-2-m-tolylbenzo [d]oxazole; 2-(4-isobutoxyphenyl)-3-(naphthalen-2-yl)-2,3-dihydroquinazolin-4 (1H)-one; N-(2-(2-(4-fluorophenyl)-2-oxocthylthio)-4-oxoquinazolin-3(4H)-yl) benzamide; 2-(4-chlorophenyl)-4-(4-methoxyphenyl)-1,4-dihydrobenzo[4,5]imidazo[1,2-a]pyrimidinc; 2-(3-pyridyl)-4-(4-bromophenyl)-1,4-dihydrobenzo[4,5]imidazo[1,2-a]pyrimidine; N-sec-butyl-1,7,7-trimethyl-9-oxo-8,9-dihydro-7H-furo[3,2-f]chromene-2-carboxamide; N-(3-carbamoyl-5,6-dihydro-4H-cyclopenta[b]thiophen-2-yl)benzofuran-2-carboxamide; 3-chloro-N-(5-chloropyridin-2-yl)benzo[b]thiophene-2-carboxamide; 3-chloro-N-((tetrahydrofuran-2-yl)methyl)benzo[b]thiophene-2-carboxamide; N-(3-(5-chloro-3-methylbenzo[b]thiopen-2-yl)-1H-pyrazol-5-yl)acetamide; 2-(naphthalen-2-yl)-1H-indole; 2-(pyridin-2-yl)-1H-indole; N-(2-chlorophenyl)-2-(1H-indole-3-yl)-2-oxoacetamide; 2-m-tolylquinoline; 2-(4-(2-methoxyphenyl)piperazin-1-yl) quinolone; 2-(1H-benzo[d][1,2,3]triazol-1-yl)-N-(2,3-dihydro-1H-inden-2-yl)acetamide; 1-phenethyl-1H-benzo[d][1,2,3] triazole; 7-(4-fluorobenzyloxy)-2H-chromen-2-one; N-(2,4-dichlorophenyl)-8-methoxy-2H-chromene-3-carboxamide; N-(3-chlorophenyl)-8-methyl-3,4-dihydroquinoline-1(2H)-carbothioamide; 7-methoxy-5-methyl-2-phenyl-4H-chromen-4-one; 2-(3,4-dimethylphenyl)quinoxaline; 4-bromo-N-(5-chloropyridin-2-yl)benzamide; 3-amino-6,7,8,9-tetrahydro-5H-cyclohepta[e]thieno[benzimidamide; N,N-diethyl-6-methoxythieno [2,3-b]quinoline-2-carboxamide; 6-(4-methoxyphenyl)-1,2,3,4-tetrahydro-1,5-naphthyridine; 5-bromo-N-(2-(phenylthio)ethyl)nicotinamide; N-(6-methylpyridin-2-yl)-2,3-dihydrobenzo[b][1,4]dioxine-6-carboxamide; 2-(4-methylbenzylthio)xazolo [4,5-b] pyridine; N-(2-methoxycthyl)-5-p-tolylpyrimidin-2-amine; 4-(5-(benzo[b]thiophen-2-yl) pyrimidin-2-yl)morpholino; 4-(5-(4-fluorophenyl)pyrimidin-2-yl)morpholino; N-(4-bromo-3-methylphenyl)quinazoline-4-amine; N-(4-methoxyphenyl) quinazolin-4-amine; N-(3-methoxyphenyl)-9H-purin-6-amine; N,N-diethyl-1-m-tolyl-1H-pyrazolo[3,4-d]pyrimidin-4-aminc; (5-(4-bromophenyl)furan-2-yl)(morpholino) methanone; (Z)-4-bromo-N′-(furan-2-carbonyloxy)benzimidamide; N-(4-iodophenyl)furan-2-carboxamide; 5-(5-(2,4-difluorophenyl)furan-2-yl)-1-(methylsulfonyl)-1H-pyrazole; 1-(3-amino-5-(4-tert-butylphenyl)thiophen-2-yl) ethanone; N-(3-cayano-4,5,6,7-tetrahydrobenzo[b]thiophen-2-yl)-2-fluorobenzamide; N-(5-chloropyridin-2-yl)thiophene-2-carboxamide; N-(2-(4-fluorophenoxy)ethyl)thiophene-2-carboxamide; 2,5-dimethyl-N-phenyl-1-(thiophen-2-ylmethyl)-1H-pyrrole-3-carboxamide; N-(3-cyanothiophen-2-yl)-4-isopropoxybenzamide; 2-(4-methoxyphenoxy)-N-(thiazol-2-yl) acctamide; 4-(4-methoxyphenyl)-N-(3-methylpyridin-2-yl) thiazol-2-amine 4-(biphenyl-4-yl) thiazol-2-amine; 4-(4-(4-methoxyphenyl)thiazol-2-yl)-3-methylisoxazol-5-amine; N-(2-methoxyphenyl)-4-phenylthiazol-2-amine; 1-(4-amino-2-(m-tolylamino)thiazol-5-yl)-2-methylpropan-1-one; 4-(4-chlorophenyl)-1-(5H-pyrimido[5,4-b]indol-4-yl)-1H-pyrazol-3-amine; 2-(4-chlorophenyl)-6-ethyl-5-methylpyrazolo[1,5-a]pyrimidin-7(4H)-one; 5-methoxy-2-(5-phenyl-1H-pyrazol-3-yl)phenol; (3-(4-bromophenyl)-1-phenyl-1H-pyrazol-4-yl)methanol; N-(2,5-dichlorophenyl)-1-ethyl-1H-pyrazole-3-carboxamide; 4-chloro-1-methyl-N-(2-oxo-2-phenylethyl)-1H-pyrazole-3-carboxamide; N-(3-(5-tert-butyl-2-methylfuran-3-yl)-1H-pyrazole-5-yl)benzamide; N-(5-methylisoxazol-3-yl)benzo[d][1,3]dioxole-5-carboxamide; (5-(4-bromophenyl)isoxazole-3-yl)(morpholino)methanone; N-(4-bromophenyl)-5-isopropylisoxazole-3-carboxamide; 5-((4-chloro-2-methylphenoxy)methyl)-3-(pyridin-4-yl)-1,2,4-oxadiazole; 5-(2-methoxyphenyl)-3-p-tolyl-1,2,4-oxadiazole; 5-(phenoxymethyl)-3-(pyridin-3-yl)-1,2,4-oxadiazole; 5-(2-chloro-4-methylphenyl)-3-(pyridin-3-yl)-1,2,4-oxadiazole; 3-(2-chlorophenyl)-5-p-tolyl-1,2,4-oxadiazole; 5-(piperidin-1-ylmethyl)-3-p-toyl-1,2,4-oxadiazole; 5-(4-bromophenyl)-3-(pyridin-3-yl)-1,2,4-oxadiazole; 5-(2-bromophenyl)-3-(4-bromophenyl)-1,2,4-oxadiazole; 5-(2-bromo-5-methoxyphenyl)-3-(thiophenyl-2-yl)-1,2,4-oxadiazole; 3-(2-fluorophenyl)-N-(3-(piperidin-1-yl) propyl)-1,2,4-oxadiazol-5-amine; 2-(2-chlorobenzoyl)-N-(4-fluorophenyhydrazinecarbothioamide; 2-(methylamino)-N-phenethylbenzamide; 4-tert-butyl-N-((tetrahydrofuran-2-yl) methyl) benzamide; -chlorophenyl)-4,5-dihydro-1H-1,2,4-triazole-5-yl)-N,N-dimethylaniline; 7-methoxy-2-(4-methoxyphenyl)-1,10b-dihydrospiro[benzo[e]pyrazolo[1,5-c][1,3]oxazine-5,1′-cyclohexane]; 6-oxo-2-(4-(3-(trifluoromethyl)phenoxy)phenyl)-1,4,5,6-tetrahydropyridine-3-carbonitrile; 6-(4-methoxyphenyl)imidazo[2,1-b]thiazole; 2-(2-bromophenoxy)-N-(4H-1,2,4-triazol-3-yl) acetamide; 1-(indolin-1-yl)-2-phenoxyethanone; 2-(4-chlorophenyl)-6,7,8,9-tetrahydrobenzo[e]imidazo [1,2-b][1,2,4]triazine; and pharmaceutically acceptable salts thereof.

### 2. Delivery of Agents for Modulating c-myc, Notch and Atoh1

### Delivery of Proteins, Activators and Inhibitors

The method of delivery of modulators of c-myc, Notch or Atoh1 activity will depend, in part, upon whether the hair cells or supporting cells are being contacted with the agents of interest in vivo or ex vivo. In the in vivo approach, the agents are delivered into the inner car of a mammal. In the ex vivo approach, cells are contacted with the agents ex vivo. The resulting hair cells can then be transplanted into the inner ear of a recipient using techniques known and used in the art.

In certain embodiments, c-myc activity is increased by administering c-myc protein or a c-myc activator in the inner ear of a recipient to give, for example, a final concentration of greater than about 30 μM, for example, in the range of about 30 μM to about 1000 μM. In certain embodiments, the c-myc protein or c-myc activator can be administered in an amount sufficient to give a final concentration of greater than about 30 μM. For example, the c-myc protein or c-myc activator may be administered in an amount sufficient to give a final concentration in the range from about 30 μM to about 1000 μM, 50 μM to about 1000 μM, 80 μM to about 1000 μM, about 100 μM to about 1000 μM, about 150 μM to about 1000 μM, from about 200 μM to about 800 μM, or from about 200 μM to about 600 μM.

In other embodiments, c-myc protein or a c-myc activator is administered at a dose from about 0.025 mg to about 4 mg, from about 0.035 mg to about 2 mg, from about 0.05 mg to about 2 mg, from about 0.1 mg to about 2 mg, from about 0.2 mg to about 1 mg, or from about 0.2mg to about 0.8 mg of the c-myc protein or c-myc activator can be administered locally to the inner car of a mammal. In one embodiment, 0.5 mg of c-myc protein or c-myc activator is administered locally to the inner ear. In certain other embodiments, from about 0.05 mg to about 2 mg, from about 0.2 mg to about 2 mg, from about 0.05 mg to about 1.5 mg, from about 0.15 mg to about 1.5 mg, from about 0.4 mg to about 1 mg, or from about 0.5 mg to about 0.8 mg of c-myc protein or c-myc activator can be administered locally to the inner car of a mammal.

In certain embodiments, Notch activity is increased by administering a Notch protein, a NICD protein or a Notch activator to an inner ear of a recipient to give a final concentration of greater than about 30 μM, for example, in the range of about 30 μM to about 1000 μM. In certain embodiments, a Notch protein, NICD protein or Notch activator can be administered in an amount sufficient to give a final concentration of greater than about 30 μM. For example, the Notch protein, NICD protein or Notch activator may be administered in an amount sufficient to give a final concentration in the range from about 30 μM to about 1000 μM, 50 μM to about 1000 μM, 80 μM to about 1000 μM, about 100 μM to about 1000 μM, about 150 μM to about 1000 M, from about 200 μM to about 800 μM, or from about 200 μM to about 600 μM.

In other embodiments, Notch protein, NICD protein or Notch activator is administered at a dose from about 0.025 mg to about 4 mg, from about 0.035 mg to about 2 mg, from about 0.05 mg to about 2 mg, from about 0.1 mg to about 2 mg, from about 0.2 mg to about 1 mg, or from about 0.2 mg to about 0.8 mg of the Notch protein, NICD protein or Notch activator can be administered locally to the inner ear of a mammal. In one embodiment, 0.5 mg of Notch protein, NICD protein or Notch activator is administered locally to the inner car of a mammal. In certain other embodiments, from about 0.05 mg to about 2 mg, from about 0.2 mg to about 2 mg, from about 0.05 mg to about 1.5 mg, from about 0.15 mg to about 1.5 mg, from about 0.4 mg to about 1 mg, or from about 0.5 mg to about 0.8 mg of Notch protein, NICD protein or Notch activator can be administered locally to the inner car of a mammal.

In certain embodiments, after cell proliferation has occurred, Notch activity is inhibited by administering a Notch inhibitor. A Notch inhibitor can be administered to give a final concentration of greater than about 30 μM, for example, in the range of about 30 μM to about 1000 μM. In certain embodiments, a Notch inhibitor can be administered in an amount sufficient to give a final concentration of greater than about 30 μM. For example, the Notch inhibitor may be administered in an amount sufficient to give a final concentration in the range from about 30 μM to about 1000 μM, 50 μM to about 1000 μM, 80 μM to about 1000 μM, about 100 μM to about 1000 μM, about 150 μM to about 1000 μM, from about 200 μM to about 800 μM, or from about 200 μM to about 600 μM. In certain embodiments, the Notch inhibitor is administered in an amount sufficient to give a final concentration of about 400 μM.

In other embodiments, a Notch inhibitor is administered at a dose from about 0.025 mg to about 4 mg, from about 0.035 mg to about 2 mg, from about 0.05 mg to about 2 mg, from about 0.1 mg to about 2 mg, from about 0.2 mg to about 1 mg, or from about 0.2 mg to about 0.8 mg of the Notch inhibitor can be administered locally to the inner ear of a mammal. In one embodiment, 0.5 mg of Notch inhibitor is administered locally to the inner ear of a mammal. In certain other embodiments, from about 0.05 mg to about 2 mg, from about 0.2 mg to about 2 mg, from about 0.05 mg to about 1.5 mg, from about 0.15 mg to about 1.5 mg, from about 0.4 mg to about 1 mg, or from about 0.5 mg to about 0.8 mg of Notch inhibitor can be administered locally to the inner car of a mammal. In certain embodiments, about 0.7 mg Notch inhibitor is administered locally to the inner car of a mammal.

In certain embodiments, Atoh1 activity is increased by administering Atoh1 protein or an Atoh1 activator in the inner ear of a recipient to give, for example, a final concentration of greater than about 30 μM, for example, in the range of about 30 μM to about 1000 μM. In certain embodiments, the Atoh1 protein or Atoh1 activator can be administered in an amount sufficient to give a final concentration of greater than about 30 μM. For example, the Atoh1 protein or Atoh1 activator may be administered in an amount sufficient to give a final concentration in the range from about 30 μM to about 1000 μM, 50 μM to about 1000 μM, 80 μM to about 1000 μM, about 100 μM to about 1000 μM, about 150 μM to about 1000 μM, from about 200 μM to about 800 μM, or from about 200 μM to about 600 μM.

In other embodiments, Atoh1 protein or a Atoh1 activator is administered at a dose from about 0.025 mg to about 4 mg, from about 0.035 mg to about 2 mg, from about 0.05 mg to about 2 mg, from about 0.1 mg to about 2 mg, from about 0.2 mg to about 1 mg, or from about 0.2 mg to about 0.8 mg of the Atoh1 protein or Atoh1 activator can be administered locally to the inner car of a mammal. In one embodiment, 0.5 mg of Atoh1 protein or Atoh1 activator is administered locally to the inner ear. In certain other embodiments, from about 0.05 mg to about 2 mg, from about 0.2 mg to about 2 mg, from about 0.05 mg to about 1.5 mg, from about 0.15 mg to about 1.5 mg, from about 0.4 mg to about 1 mg, or from about 0.5 mg to about 0.8 mg of Atoh1 protein or Atoh1 activator can be administered locally to the inner car of a mammal.

### Delivery of DNA

In some aspects, the activity of c-myc, Notch or AtohI can be increased in a target cell using expression constructs known in the art, e.g., naked DNA constructs, DNA vector based constructs, and/or viral vector and/or viral based constructs to express nucleic acids encoding a desired c-myc, Notch or Atoh1 protein. In certain embodiments, a single DNA construct expressing c-myc and Notch or NICD as two separate genes can be delivered into the inner car of a subject. In certain embodiments, a single DNA construct expressing c-myc and Notch or NICD and Atoh1 as three separate genes can be delivered into the inner ear of a subject.

Exemplary expression constructs can be formulated as a pharmaceutical composition, e.g., for administration to a subject.

DNA constructs and the therapeutic use of such constructs are well known to those of skill in the art (see, e.g., Chiarella et al. (2008) RECENT PATENTS ANTI-INFECT. DRUG DISC. 3:93-101; Gray et al. (2008) EXPERT PIN. BIOL. THER. 8:911-922; Melman et al. (2008) HUM. GENE THER. 17:1165-1176). Naked DNA constructs typically include one or more therapeutic nucleic acids (e.g., DNA encoding c-myc and/or Notch) and a promoter sequence. A naked DNA construct can be a DNA vector, commonly referred to as pDNA. Naked DNA typically do not integrate into chromosomal DNA. Generally, naked DNA constructs do not require, or are not used in conjunction with, the presence of lipids, polymers, or viral proteins. Such constructs may also include one or more of the non-therapeutic components described herein.

DNA vectors are known in the art and typically are circular double stranded DNA molecules. DNA vectors usually range in size from three to five kilo-base pairs (e.g., including inserted therapeutic nucleic acids). Like naked DNA, DNA vectors can be used to deliver and express one or more therapeutic proteins in target cells. DNA vectors do not integrate into chromosomal DNA.

Generally, DNA vectors include at least one promoter sequence that allows for replication in a target cell. Uptake of a DNA vector may be facilitated by combining the DNA vector with, for example, a cationic lipid, and forming a DNA complex. Typically, viral vectors are double stranded circular DNA molecules that are derived from a virus. Viral vectors typically are larger in size than naked DNA and DNA vector constructs and have a greater capacity for the introduction of foreign (i.e., not virally encoded) genes. Like naked DNA and DNA vectors, viral vectors can be used to deliver and express one or more therapeutic nucleic acids in target cells. Unlike naked DNA and DNA vectors, certain viral vectors stably incorporate themselves into chromosomal DNA. Typically, viral vectors include at least one promoter sequence that allows for replication of one or more vector encoded nucleic acids, e.g., a therapeutic nucleic acid, in a host cell. Viral vectors may optionally include one or more non-therapeutic components described herein. Advantageously, uptake of a viral vector into a target cell does not require additional components, e.g., cationic lipids. Rather, viral vectors transfect or infect cells directly upon contact with a target cell.

The approaches described herein include the use of retroviral vectors, adenovirus-derived vectors, and/or adeno-associated viral vectors as recombinant gene delivery systems for the transfer of exogenous genes in vivo, particularly into humans. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals.

Viruses that are used as transduction agents of DNA vectors and viral vectors such as adenoviruses, retroviruses, and lentiviruses may be used in practicing the present invention. Illustrative retroviruses include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus. As used herein, the term “lentivirus” refers to a group (or genus) of complex retroviruses. Illustrative lentiviruses include, but are not limited to: HIV (human immunodeficiency virus; including HIV type 1, and HIV type 2); visna-macdi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In certain embodiments, an adenovirus can be used in accordance with the methods described herein. The genome of an adenovirus can be manipulated such that it encodes and expresses a gene product of interest but is inactivated in terms of its ability to replicate in a normal lytic viral life cycle. Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors.

Adeno-associated virus is a naturally occurring defective virus that requires another virus, such as an adenovirus or a herpes virus, as a helper virus for efficient replication and a productive life cycle. It is also one of the few viruses that may integrate its DNA into non-dividing cells, and exhibits a high frequency of stable integration.

In various embodiments, one or more viral vectors that expresses a therapeutic transgene or transgenes encoding a polypeptide or polypeptides of the invention (e.g., Atoh1, Notch, c-myc) is administered by direct injection to a cell, tissue, or organ of a subject, in vivo.

In various other embodiments, cells are transduced in vitro or ex vivo with such a vector encapsulated in a virus, and optionally expanded ex vivo. The transduced cells are then administered to the inner ear of a subject. Cells suitable for transduction include, but are not limited to stem cells, progenitor cells, and differentiated cells. In certain embodiments, the transduced cells are embryonic stem cells, bone marrow stem cells, umbilical cord stem cells, placental stem cells, mesenchymal stem cells, neural stem cells, liver stem cells, pancreatic stem cells, cardiac stem cells, kidney stem cells, hematopoietic stem cells, inner ear hair cells, iPS cells, inner ear supporting cells, cochlear cells, or utricular cells.

In particular embodiments, host cells transduced with viral vector of the invention that expresses one or more polypeptides, are administered to a subject to treat and/or prevent an auditory disease, disorder, or condition. Other methods relating to the use of viral vectors, which may be utilized according to certain embodiments of the present invention, can be found in, e.g., Kay (1997) CHEST 111 (6 Supp.):138S-142S; Ferry et al. (1998) HUM. GENE THER. 9:1975-81;Shiratory et al. (1999) LIVER 19:265-74; Oka et al. (2000) CURR. OPIN. LIPIDOL. 11:179-86; Thule et al. (2000) Gene Ther. 7:1744-52; Yang (1992) CRIT. REV. BIOTECHNOL. 12:335-56; Alt (1995) J. HEPATOL. 23:746-58; Brody et al. (1994) ANN. N. Y. ACAD. SCI. 716:90-101; Strayer. (1999) EXPERT OPIN. INVESTIG. DRUGS 8:2159-2172; Smith-Arica et al. (2001) CURR. CARDIOL. REP. 3:43-49; and Lec et al. (2000) NATURE 408:483-8.

In some embodiments of the invention, it may be desirable to use a cell, cell type, cell lineage or tissue specific expression control sequence to achieve cell type specific, lineage specific, or tissue specific expression of a desired polynucleotide sequence, for example, to express a particular nucleic acid encoding a polypeptide in only a subset of cell types, cell lineages, or tissues, or during specific stages of development. Illustrative examples of cell, cell type, cell lineage or tissue specific expression control sequences include, but are not limited to: an Atoh1 enhancer for all hair cells (see, e.g., FIG. 24); a Pou4f3 promoter for all hair cells (see, e.g., FIG. 25); a Myo7a promoter for all hair cells (see, e.g., FIG. 26); a Hes5 promoter for vestibular supporting cells and cochlear inner phalangeal cells, Deiters cells and Pillar cells (see, e.g., FIG. 27); and GFAP promoter for vestibular supporting cells and cochlear inner phalangeal cells, Deiters cells and Pillar cells (see, e.g., FIG. 28).

Certain embodiments of the invention provide conditional expression of a polynucleotide of interest. For example, expression is controlled by subjecting a cell, tissue, organism, etc., to a treatment or condition that causes the polynucleotide to be expressed or that causes an increase or decrease in expression of the polynucleotide encoded by the polynucleotide of interest. Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, GENE, 323:67), the cumate inducible gene switch (WO 2002/088346), tetracycline-dependent regulatory systems, etc.

Conditional expression can also be achieved by using a site specific DNA recombinase. According to certain embodiments of the invention the vector comprises at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy (1993) CURRENT OPINION IN BIOTECHNOLOGY 3:699-707), or mutants, derivatives (e.g., fusion proteins containing the recombination protein sequences or fragments thereof), fragments, and variants thereof. Illustrative examples of recombinases suitable for use in particular embodiments of the present invention include, but are not limited to: Cre, Int, IHF, Xis, Flp, Fis, Hin, Gin, OC31, Cin, Tn3 resolvase, TndX, XerC, XerD, TnpX, Hjc, Gin, SpCCEl. and ParA.

The vectors may comprise one or more recombination sites for any of a wide variety of site specific recombinases. It is to be understood that the target site for a site specific recombinase is in addition to any site(s) required for integration of a vector (e.g., a retroviral vector or lentiviral vector).

In certain embodiments, vectors comprise a selection gene, also termed a selectable marker. Typical selection genes encode proteins that (a) confer resistance to antibiotics or other toxins, e.g., ampicillin, neomycin, hygromycin, methotrexate, Zeocin, Blastocidin, or tetracycline, (b) complement auxotrophic deficiencies, or (c) supply critical nutrients not available from complex media, e.g., the gene encoding D-alanine racemase for Bacilli. Any number of selection systems may be used to recover transformed cell lines. These include, but are not limited to, the herpes simplex virus thymidine kinase (Wigler et al., (1977) CELL 11:223-232) and adenine phosphoribosyltransferase (Lowy et al., (1990) CELL 22:817-823) genes which can be employed in tk-or aprt-cells, respectively.

All the molecular biological techniques required to generate an expression construct described herein are standard techniques that will be appreciated by one of skill in the art.

In certain embodiments, DNA delivery may occur auricularly, parenterally, intravenously, intramuscularly, or even intraperitoneally as described, for example, in U.S. Pat. Nos. 5,543,158; 5,641,515; and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

In certain embodiments, DNA delivery may occur by use of liposomes, nanocapsules, microparticles, microspheres, lipid particles, vesicles, optionally mixing with cell penetrating polypeptides, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

Exemplary formulations for ex vivo DNA delivery may also include the use of various transfection agents known in the art, such as calcium phosphate, electroporation, heat shock and various liposome formulations (i.e., lipid-mediated transfection). Particular embodiments of the invention may comprise other formulations, such as those that are well known in the pharmaceutical art, and are described, for example, in Remington: The Science and Practice of Pharmacy, 20th Edition. Baltimore, MD: Lippincott Williams & Wilkins, 2000.

### Duration of Delivery

The duration of c-myc, Notch and Atoh1 activation can be varied to achieve a desired result. For example, it may be beneficial to expose a target cell to a c-myc protein or c-myc activator and a Notch protein, NICD protein, or a Notch activator for one to six days, one week, two weeks, three weeks, one month, three months, six months, nine months, one year, two years or more. Alternatively, when c-myc is increased by constitutive activation (e.g., using an adenovirus to overexpress c-myc), the duration of increased c-myc activity can be controlled by administering a c-myc inhibitor following administration of a myc protein or a myc activator. Inhibiting c-myc activity after a period of increased c-myc activity can be used to control proliferation, promote cell survival, and avoid tumorigenesis.

Similarly, the duration of increased Notch activity can be controlled by administering a Notch inhibitor, as discussed above, following administration of a Notch protein, NICD protein, or a Notch activator.

### Route of Administration and Formulation

The route of administration will vary depending on the disease being treated. Hair cell loss, sensorineural hearing loss, and vestibular disorders can be treated using direct therapy using systemic administration and/or local administration. In certain embodiments, the route of administration can be determined by a subject's health care provider or clinician, for example following an evaluation of the subject.

The invention provides (i) a composition for use in proliferating or regenerating a cochlear or a utricular hair cell, (ii) a composition for use in proliferating or regenerating a cochlear or a utricular supporting cell, (iii) a composition for use in reducing the loss of, maintaining, or promoting hearing in a subject, and (iv) a composition for use in reducing the loss of, maintaining, or promoting vestibular function in a subject. Accordingly, the invention provides a first composition comprising an agent, for example, each of the agents discussed hereinabove, for example, an agent that increases c-myc activity and/or an agent that increases Notch activity within a hair or supporting cell, either alone or in combination with a pharmaceutically acceptable carrier for use in each of the foregoing approaches. In addition, the invention provides a second composition comprising an agent, for each of the agents discussed hereinabove, for example, an agent that reduces or inhibits c-myc activity and/or an agent that reduces or inhibits Notch activity within a hair or supporting cell, either alone or in combination with in a pharmaceutically acceptable carrier for use in each of the foregoing approaches. When supporting cells are regenerated, the invention provides a third composition comprising an agent, for example, an agent for increasing Atoh1 activity, to induce transdifferentiation of a proliferated supporting cell into a hair cell.

In certain embodiments, a c-myc protein or c-myc activator and a Notch protein, NICD protein or Notch activator can be formulated as a pharmaceutical composition containing the appropriate carriers and/or excipients.

The c-myc protein or activator and/or the Notch protein, NICD protein, or Notch activator, and/or the Atoh1 protein or activator can be solubilized in a carrier, for example, a viscoelastic carrier, that is introduced locally into the inner ear. In other embodiments, the c-myc protein or activator and/or the Notch protein, NICD protein, or Notch activator, and/or Atoh1 protein or activator can be solubilized in a liposome or microsphere. Methods for delivery of a drug or combination of drugs in liposomes and/or microspheres are well-known in the art.

In addition, it is contemplated that the c-myc protein or activator and/or the Notch protein, NICD protein, or Notch activator, and/or Atoh1 protein or activator can be formulated so as to permit release of one or more proteins and/or activators over a prolonged period of time. A release system can include a matrix of a biodegradable material or a material, which releases the incorporated active agents. The active agents can be homogeneously or heterogeneously distributed within a release system. A variety of release systems may be useful in the practice of the invention, however, the choice of the appropriate system will depend upon the rate of release required by a particular drug regime. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic.

In certain embodiments, the agents can be administered to a subject, e.g., a subject identified as being in need of treatment for hair cell loss, using a systemic route of administration. Systemic routes of administration can include, but are not limited to, parenteral routes of administration, e.g., intravenous injection, intramuscular injection, and intraperitoneal injection; enteral routes of administration, e.g., administration by the oral route, lozenges, compressed tablets, pills, tablets, capsules, drops (e.g., car drops), syrups, suspensions and emulsions; rectal administration, e.g., a rectal suppository or enema; a vaginal suppository; a urethral suppository; transdermal routes of administration; and inhalation (e.g., nasal sprays).

Alternatively or in addition, the agents can be administered to a subject, e.g., a subject identified as being in need of treatment for hair cell loss, using a local route of administration. Such local routes of administration include administering one or more compounds into the car of a subject and/or the inner ear of a subject, for example, by injection and/or using a pump.

In certain embodiments, the agents may be injected into the car (e.g., auricular administration), such as into the luminae of the cochlea (e.g., the Scala media, Sc vestibulac, and Sc tympani). For example, the agents can be administered by intratympanic injection (e.g., into the middle car), and/or injections into the outer, middle, and/or inner ear. Such methods are routinely used in the art, for example, for the administration of steroids and antibiotics into human cars. Injection can be, for example, through the round window of the car or through the cochlea capsule.

In other embodiments, the agents can be delivered via nanoparticles, for example, protein-coated nanoparticles. Nanoparticles can be targeted to cells of interest based on cell-type specific receptor affinity for ligands coating the nanoparticles. The dosage of the agent can be modulated by regulating the number of nanoparticles administered per dose.

Alternatively, the agent may be administered to the inner ear using a catheter or pump. A catheter or pump can, for example, direct the agent into the cochlea luminae or the round window of the car. Exemplary drug delivery systems suitable for administering one or more compounds into an car, e.g., a human car, are described in U.S. Patent Publication No. 2006/0030837 and U.S. Pat. No. 7,206,639. In certain embodiments, a catheter or pump can be positioned, e.g., in the car (e.g., the outer, middle, and/or inner car) of a subject during a surgical procedure.

Alternatively or in addition, the agents can be delivered in combination with a mechanical device such as a cochlea implant or a hearing aid, which is worn in the outer car. An exemplary cochlea implant that is suitable for use with the present invention is described in U.S. Patent Publication No. 2007/0093878.

In certain embodiments, the modes of administration described above may be combined in any order and can be simultaneous or interspersed. For example, the agents may be administered to a subject simultaneously or sequentially. It will be appreciated that when administered simultaneously, the agents may be in the same pharmaceutically acceptable carrier (e.g., solubilized in the same viscoelastic carrier that is introduced into the inner ear) or the two agents may be dissolved or dispersed in separate pharmaceutical carriers, which are administered at the same time. Alternatively, the agents may be provided in separate dosage forms and administered sequentially.

Alternatively or in addition, the agents may be administered according to any of the Food and Drug Administration approved methods, for example, as described in CDER Data Standards Manual, version number 004 (which is available at fda.give/cder/dsm/DRG/drg00301.htm).

### 3. Delivery of Agents to Hair Cells and Supporting Cells Ex Vivo

It is understood that the concepts for delivering agents of interest to hair cells and supporting cells in vivo can also apply to the delivery of the agents of interest to hair cells and supporting cells ex vivo. The hair cells and supporting cells can be harvested and cultured using techniques known and used in the art. The agents (protein expression vectors, activators and inhibitors (for example, as discussed above)) can then be contacted with the cultured hair cells or supporting cells to induce the cells to reenter the cell cycle, and proliferate. Thereafter, once the cells have proliferated, the c-myc and Notch activities can be inhibited using appropriate inhibitors, for example, those discussed above. The resulting hair cells can then be maintained in culture for any number of uses, including, for example, to study the biological, biophysical, physiological and pharmacological characteristics of hair cells and/or supporting cells. Alternatively, the resulting hair cells can then be implanted in to the inner car of a recipient using standard surgical procedures.

In certain embodiments, suitable cells can be derived from a mammal, such as a human, mouse, rat, pig, sheep, goat, or non-human primate. In certain embodiments, the cells can be harvested from the inner car of a subject, and cells can be obtained from the cochlea organ of Corti, the modiolus (center) of the cochlea, the spiral ganglion of the cochlea, the vestibular sensory epithelia of the saccular macula, the utricular macula, or the cristae of the semicircular canals. Alternatively or in addition, methods include obtaining tissue from the inner car of the animal, where the tissue includes at least a portion of the utricular maculae.

Tissue isolated from a subject can be suspended in a neutral buffer, such as phosphate buffered saline (PBS), and subsequently exposed to a tissue-digesting enzyme (e.g., trypsin, leupeptin, chymotrypsin, and the like) or a combination of enzymes, or a mechanical (e.g., physical) force, such as trituration, to break the tissue into smaller pieces. Alternatively, or in addition, both mechanisms of tissue disruption can be used. For example, the tissue can be incubated in about 0.05 % enzyme (e.g., about 0.001%, 0.01%, 0.03%, 0.07%, or 1.0% of enzyme) for about 5, 10, 15, 20, or 30 minutes, and following incubation, the cells can be mechanically disrupted. The disrupted tissue can be passed through a device, such as a filter or bore pipette, that separates a stem cell or progenitor cell from a differentiated cell or cellular debris. The separation of the cells can include the passage of cells through a series of filters having progressively smaller pore size. For example, the filter pore size can range from about 80 μm or less, about 70 μm or less, about 60 μm or less, about 50 μm or less, about 40 μm or less, about 30 μm or less, about 35 um or less, or about 20 μm or less.

Partially and/or fully differentiated cells, e.g., generated by the methods described above, can be maintained in culture for a variety of uses, including, for example, to study the biological, biophysical, physiological and pharmacological characteristics of hair cells and/or supporting cells. Cell cultures can be established using inner ear cells from subjects with hearing loss and/or loss in vestibular function to develop potential treatments (e.g., to screen for drugs effective in treating the hearing loss and/or loss in vestibular function). Further, the methods of the present invention can be used in combination with induced pluripotent stem (iPS) cell technology to establish cell lines (e.g., hair cell lines and/or supporting cell lines). For example, fibroblasts from a subject with hearing loss can be induced to form iPS cells using known techniques (see, for example, Oshima et al. (2010) CELL 141(4):704-716). However, because the numbers of cells generated using iPS cell technology is limited, the methods provided herein can be used in combination with iPS cell technology to produce sufficient numbers of cells to establish cell lines (e.g., hair cell lines and/or supporting cell lines).

Partially and/or fully differentiated cells, e.g., generated by the methods described above, can be transplanted or implanted, such as in the form of a cell suspension, into the car by injection, such as into the luminae of the cochlea. Injection can be, for example, through the round window of the car or through the bony capsule surrounding the cochlea. The cells can be injected through the round window into the auditory nerve trunk in the internal auditory meatus or into the scala tympani. In certain embodiments, the cells described herein can be used in a cochlea implant, for example, as described in U.S. Patent Publication No. 2007/0093878.

To improve the ability of transplanted or implanted cells to engraft, cells can be modified prior to differentiation. For example, the cells can be engineered to overexpress one or more anti-apoptotic genes. The Fak tyrosine kinase or Akt genes are candidate anti-apoptotic genes that can be used for this purpose; overexpression of FAK or Akt can prevent cell death in spiral ganglion cells and encourage engraftment when transplanted into another tissue, such as an explanted organ of Corti (see, for example, Mangi et al., (2003) NAT. MED. 9:1195-201). Neural progenitor cells overexpressing αᵥβ₃ integrin may have an enhanced ability to extend neurites into a tissue explant, as the integrin has been shown to mediate neurite extension from spiral ganglion neurons on laminin substrates (Aletsee et al., (2001) AUDIOL. NEUROOTOL. 6:57-65). In another example, ephrinB2 and ephrinB3 expression can be altered, such as by silencing with RNAi or overexpression with an exogenously expressed cDNA, to modify EphA4 signaling events. Spiral ganglion neurons have been shown to be guided by signals from EphA4 that are mediated by cell surface expression of ephrin-B2 and-B3 (Brors et al., (2003) J. COMP. NEUROL. 462:90-100). Inactivation of this guidance signal may enhance the number of neurons that reach their target in an adult inner car. Exogenous factors such as the neurotrophins BDNF and NT3, and LIF can be added to tissue transplants to enhance the extension of neurites and their growth towards a target tissue in vivo and in ex vivo tissue cultures. Neurite extension of sensory neurons can be enhanced by the addition of neurotrophins (BDNF, NT3) and LIF (Gillespie et al. (2010) NEUROREPORT 12:275-279).

4. Measurement of c-myc, Notch or Atoh1 Activity in Target Cells

The methods and compositions described herein can be used to induce cells, e.g., adult mammalian inner ear cells, to reenter the cell cycle and proliferate. For example, the number of hair cells can be increased about 2-, 3-, 4-, 6-, 8-, or 10-fold, or more, as compared to the number of hair cells before treatment. The hair cell can be induced to reenter the cell cycle in vivo or ex vivo. It is contemplated that using these approaches it may be possible to improve the hearing of a recipient. For example, using the methods and compositions described herein, it may be possible to improve the hearing of a recipient by at least about 5, 10, 15, 20, 40, 60, 80, or 90% relative to the hearing prior to the treatment. Tests of auditory or vestibular function also can be performed to measure hearing improvement.

Cells that have been contacted with (i) a c-myc protein or c-myc activator and/or (ii) a Notch protein, NICD protein or Notch activator, can be assayed for markers indicative of cell cycle reentry and proliferation. In one example, a cell can be assayed for incorporation of EdU (5-ethynyl-2′-deoxyuridine) followed sequentially by BrdU (5-bromo-2′-deoxyuridine) by using, for example, an anti-EdU antibody and an anti-BrdU antibody. Labelling by EdU and/or BrdU is indicative of cell proliferation. In addition, double labeling of EdU and BrdU can be used to demonstrate that a cell has undergone division at least two times. Alternatively or in addition, a cell can be assayed for the presence of phosphorylated histone H3 (Ph3) or aurora B, which are indicative of a cell that has reentered the cell cycle and is undergoing metaphase and cytokinesis.

Cell markers can also be used to determine whether a target cell, e.g., a hair cell or a supporting cell, has entered the cell cycle. Exemplary markers indicative of hair cells include Myo7a, Myo6, Prestin, Lhx3, Dner, espin, parvalbumin, and calretinin. Exemplary markers indicative of supporting cells include Sox2, S100a1, Prox1, Rps6, and Jag1. Double labeling of a cell cycle and/or proliferation marker and a cell-type molecule can be used to determine which cells have reentered the cell cycle and are proliferating.

In addition, neuronal markers, e.g., acetylated tubulin, neurofilament and CtBP2, can be used to detect neuronal structure, to determine whether proliferating hair cells are in contact with neurons. The presence of neuronal markers adjacent to or in contact with hair cells suggests that newly-generated hair cells have formed synapses with neurons (e.g., ganglion neurons) and that the hair cells are differentiated.

Where appropriate, following treatment, the subject, for example, a human subject, can be tested for an improvement in hearing or in other symptoms related to inner ear disorders. Methods for measuring hearing are well-known and include pure tone audiometry, air conduction, auditory brainstem response (ABR) and bone conduction tests. These exams measure the limits of loudness (intensity) and pitch (frequency) that a human can hear. Hearing tests in humans include behavioral observation audiometry (for infants to seven months), visual reinforcement orientation audiometry (for children 7 months to 3 years) and play audiometry for children older than 3 years. Oto-acoustic emission testing can be used to test the functioning of the cochlea hair cells, and electro-cochleography provides information about the functioning of the cochlea and the first part of the nerve pathway to the brain. In certain embodiments, treatment can be continued with or without modification or can be stopped.

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable. Moreover, two or more steps or actions may be conducted simultaneously.

### EXAMPLES

The invention is further illustrated by the following examples, which are provided for illustrative purposes only, and should not be construed as limiting the scope or content of the invention in any way.

### Example 1: In Vivo Induction of Cell Cycle Reentry in Adult Cochlear Cells via C-Myc and Notch

This example demonstrates that providing c-myc and Notch to cells of the inner ear of an adult animal can induce cell cycle reentry and cell proliferation among differentiated cochlear hair and supporting cells.

Adult mice aged between 1 and 15 months were used to investigate the potential for c-myc and Notch to induce cell cycle reentry, proliferation, differentiation, and survival among cochlear hair and supporting cells. In separate experiments, the mice used were either wild type (WT) background mice or mice harboring a LoxP-flanked NICD cassette (NICD^{flox/flox}) susceptible to Cre-mediated recombination resulting in activation of NICD expression. The NICD cassette encoded (from 5′ to 3′) an intracellular fragment of mouse Notch1 (amino acids 1749-2293,lacking the C-terminal PEST domain, see Murthaugh et al. (2003) PROC. NATL. ACAD. SCI. U.S.A. 100(25): 14920-14925.) Mice were anaesthetized and cochleostomy was performed to allow injection of adenovirus. Virus was injected via the scala media, facilitating infection of hair and supporting cells within the cochlear sensory epithelium. A mixture of adenovirus carrying a combination of either human c-myc (Ad-Myc) and CRE-GFP (Ad-Cre-GFP) expression cassettes or c-myc and NICD (Ad-NICD) expression cassettes was injected into the cochlea of either NICD^{flox/flox}or WT mice, respectively. One ear per mouse was injected, while the other car served as an uninjected control. An additional control was used in which cochlea were injected with Ad-Cre-GFP alone. Ad-Myc induced myc overexpression, Ad-NICD induced NICD overexpression, and Ad-CRE-GFP induced overexpression of CRE-GFP, recombination at loci flanked by LoxP sequences, and—in the case of NICD^{flox/flox} mice—NICD overexpression. Virus titered at 2×10¹²plaque-forming units (pfu) was mixed in equal parts, and a total of 0.6 μL virus was injected per animal. Following viral injection, 5-bromo-2-deoxyuridine (BrdU) was injected daily between 1 and 5 days.

Mice were sacrificed and cochlea were harvested at either 4, 8, 12, 35, or 60 days post-viral injection. Cochlea were dissected, fixed, and decalcified prior to whole mount immunostaining. Hair cells were identified via labeling with antibodies directed against Myo7a and espin. Supporting cells were identified via labeling with antibodies directed against Sox2. Cell cycle reentry and proliferation were assessed via labeling antibodies directed against BrdU. Nuclear labeling was achieved via DAPI exposure.

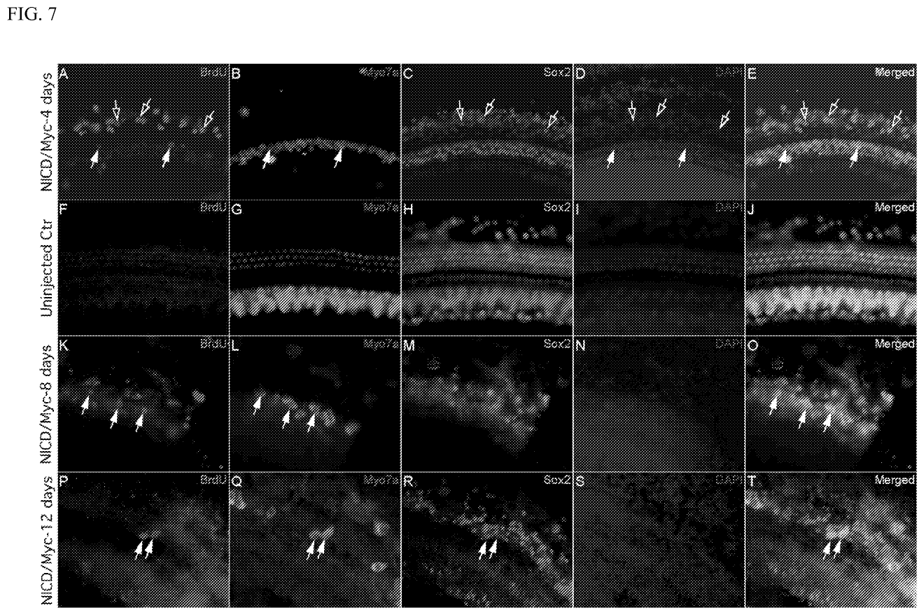

Cells of the cochlear epithelium exposed to c-myc and NICD via viral injection were analyzed to determine whether cell cycle reentry and proliferation occurred. Cochlea from NICD/lov/flox mice injected with Ad-Cre-GFP and Ad-Myc followed by BrdU administration were harvested at 4, 8, or 12 days post-virus injection and immunostained (FIG. 7). At all time points analyzed, immunostained sections revealed the presence of cycling hair cells as determined by BrdU+/Myo7a+ (FIG. 7, panels A, B, E, K, L, O, P, Q, T, closed arrows) staining. At 4 days post-injection, BrdU+/Sox2+ (FIG. 7, panels A, B, E, open arrows) staining showed that supporting cells also reentered the cell cycle in this population. These findings demonstrate that cochlear hair cells and supporting cells can be induced to reenter the cell cycle following exposure to c-myc and NICD. BrdU-labeled hair cell doublets (assumed to be daughter cells derived from the same cell division) at 12 days post-virus injection were observed, demonstrating that cells induced to reenter the cell cycle following c-Myc and NICD exposure can subsequently proliferate (FIG. 7, panels P-T, arrows). Furthermore, BrdU staining in cochlear cells was not observed in uninjected control cars at any time point (FIG. 7, panels F-J, showing 4 day time point). These observations suggest that exposing differentiated cochlear hair and supporting cells to increased c-myc and Notch activity induces cell cycle reentry within these populations.

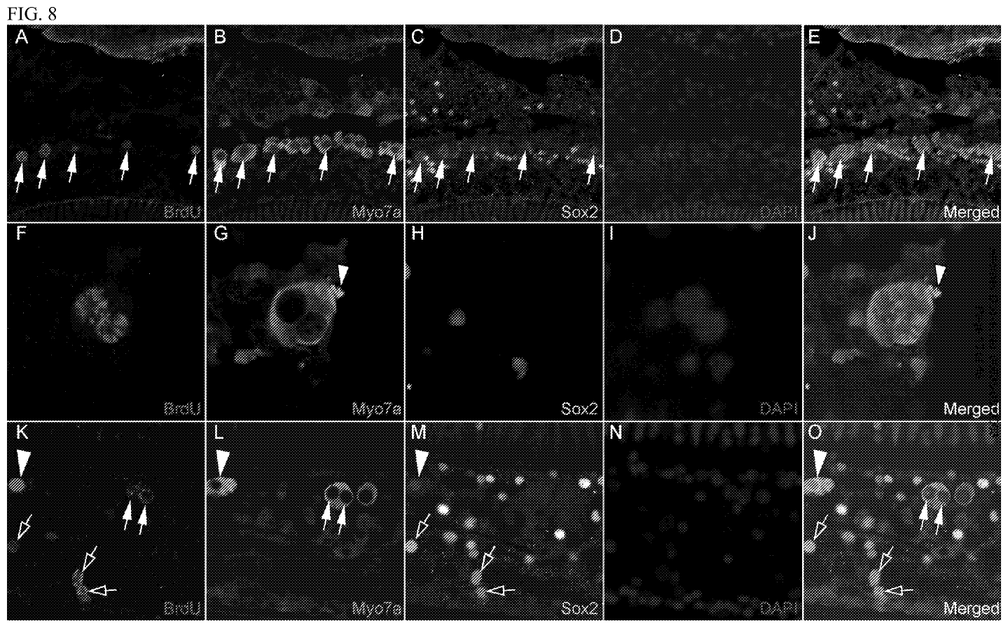

The in vivo cell survival of hair and supporting cells induced to reenter the cell cycle at more distant time points after viral injection was assessed. Cochlear tissue from NICD/lovilox mice infected with Ad-Cre-GFP and Ad-Myc virus and subsequently subjected to BrdU injection was harvested 35 days post-virus injection and immunostained to assess cell cycle reentry and survival of cycling hair and supporting cells. Analysis of stained cochlea at this time point again revealed the presence of proliferating hair and supporting cells (FIG. 8). Myo7a-positive hair cells stained positive for BrdU in cochlear epithelia subjected to BrdU labeling and harvested 35 days post-virus injection were observed (FIG. 8, panels A-E, arrows). In the same animals, BrdU-labeled Sox2-positive supporting cells were observed (FIG. 8, panels K-O, open arrows). A dividing hair cell in which Sox2 is activated by Notch is also shown (FIG. 8, panel M, arrowhead). These observations demonstrate that supporting cells and hair cells induced to reenter the cell cycle following exposure to increased c-myc and Notch activity can survive for at least 35 days in vivo. BrdU-labeled hair cells displaying stereocilia following c-Myc and NICD virus exposure at this time point were also observed (FIG. 8, panels F-J, arrowhead in panel J). This finding demonstrates that hair cells induced to reenter the cell cycle or their progeny retain physical characteristics of differentiated hair cells.

In a similar set of experiments, a mixture of Ad-Myc and Ad-NICD was injected into the scala media of WT mice followed by daily administration of BrdU from one to five days. Cochlea were harvested at time points between 2 and 35 days post-virus injection and immunostained. Immunostaining with antibodies directed against BrdU, Myo7a, and Sox2 antigens revealed the presence of double-labeled hair (BrdU+/Myo7a+) and supporting (BrdU+/Sox2+) cells in harvested cochlea. (Data not shown.) Accordingly, exposure to increased c-myc and Notch activity in differentiated hair and supporting cells of WT background also induces cell cycle reentry and proliferation.

### Example 2: In Vivo Induction of Cell Cycle Reentry in Cochlear Cells of Aged Mice via C-Myc and Notch

The following example demonstrates that providing c-myc and Notch to cells of the inner ear can also induce cell cycle reentry and cell proliferation among differentiated cochlear hair and supporting cells in aged animal subjects.

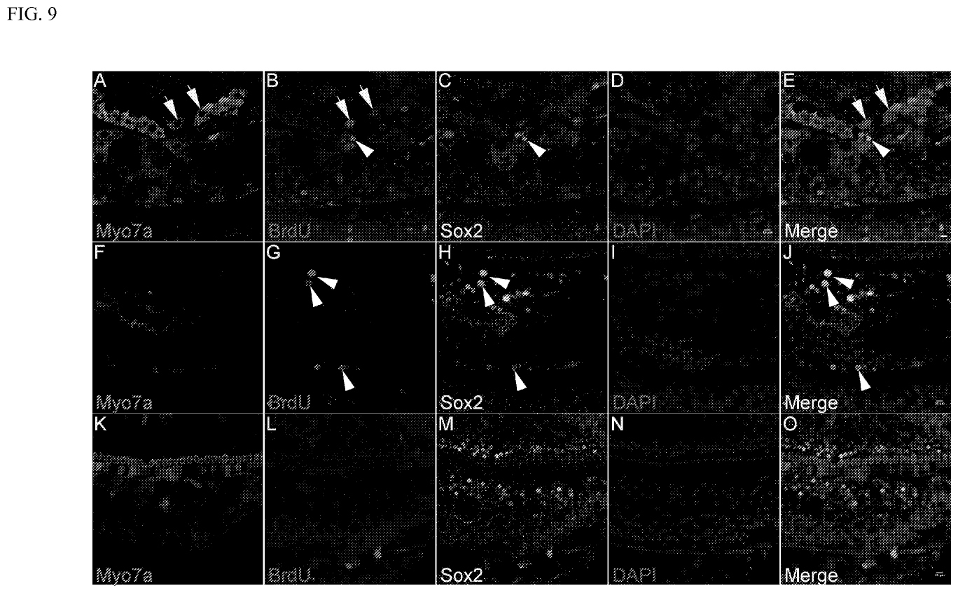

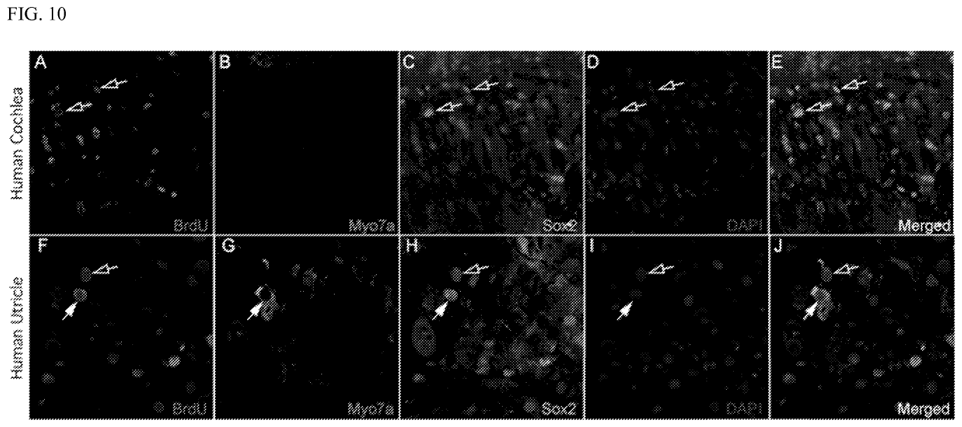

Ad-Myc and Ad-Cre-GFP were injected once into 17-month old NICD^{flox/flox} mouse cochlear scala media via cochleostomy and the animals were harvested 15 days later. 0.3 □1 of a mixture of an equal amount of Ad-Cre-GFP and Ad-Myc with a titer of 2×10¹² was injected. BrdU (50 □g/g body weight) was also injected once per day for 15 days to label cycling cells. The same protocol was used as a control, in which only Ad-Cre was injected into the cochlea. Cochlear tissue harvested following BrdU and virus injection demonstrated that cells of the aged mouse cochlea underwent cell re-entry, as evidenced by the presence of double-labeled hair (BrdU+/Myo7a+) and supporting (BrdU+/Sox2+; FIG. 9, panels A-J; arrows identify double-labeled hair cells; arrowheads identify double-labeled support cells). By contrast, no BrdU labeling was observed in Sox2+ support or Myo7a+ hair cells in 17-month old NICD^{flox/flox} control animals injected with Ad-Cre alone and subjected to the same BrdU labeling time course (FIG. 9, panels K-O).

These results demonstrate that inner ear hair and support cell proliferation can be achieved in aged mice, which suggest that similar effects can be achieved in the aged human inner car.

### Example 3: Induction of Cell Cycle Reentry in Cultured Adult Cells Harvested from Inner Ear Tissue of Various Mammals

The following example demonstrates that exposure to increased c-myc and Notch activity supports cell cycle reentry and proliferation of adult mouse, monkey and human hair and supporting cells of the inner car.

In order to investigate whether increased c-myc and Notch activity induce cell cycle reentry and proliferation in human cells, adult human cochlear and utricular tissue was collected. Samples were derived from surgeries during which such tissue was discarded. Cells were cultured in high glucose Dulbecco's modified Eagle's medium and F12 medium supplemented with N2 and B27 (Media and supplements were from Invitrogen/GIBCO/BRL, Carlsbad, CA), and 1% FBS was added.

A working viral titer of 10⁸ was used for 5 mL of culture. Cultures of harvested tissue and transduced cultured cells were contacted with a mixture of Ad-Myc and Ad-NICD, to elevate cellular levels of c-myc and NICD. Following virus exposure, the cycling cells were labeled via 3□g/ml BrdU administration to the culture. As in the in vivo studies of transduced mouse tissue, BrdU-labeled supporting (Sox2+) cells and at least one BrdU-labeled hair (Myo7a+) cell in cultured human tissue (FIG. 10) were identified.

BrdU+/Sox2+ supporting cells were identified in the cochlear cultures (FIG. 10, panels A, C, D, E) and utricular cultures (FIG. 10, panels F, H, I, J; all panels, open arrows). The cochlear cell cultures contained virtually no hair cells, so no BrdU-labeled cochlear hair cells were detected. Exposure to virus resulted in few labeled hair cells in utricular cultures, which may be the result of low infection rate of hair cells by adenovirus. However, at least one BrdU+/Myo7a+ hair cell was identified in the human utricular cultures (FIG. 10, panels F, G, I, J; closed arrow).

Similar culture-based experiments were performed utilizing harvested mouse utricle as the culture tissue. In the latter experiments, tissue was derived from either NICD^{flox/flox} or WT mice and infected with a mixture of Ad-Myc/Ad-Cre-GFP or Ad-Myc/Ad-NICD, respectively. Following viral transduction, the cells were exposed to BrdU to label the cycling cells. BrdU was added to a final concentration of 3 μg/ml. As in the human utricle culture-based experiments, BrdU-labeled hair and supporting cells in the murine cultures were observed, demonstrating that these cells can reenter the cell cycle upon exposure to increased levels of Notch and c-myc activity. Examples of BrdU-labeled hair and supporting cells were observed in these cultures, although the majority of BrdU-labeled cells were supporting cells. Based on these findings, it appears that increased c-myc and Notch activity induces cell cycle reentry and proliferation in cultured hair and supporting cells of the inner car.

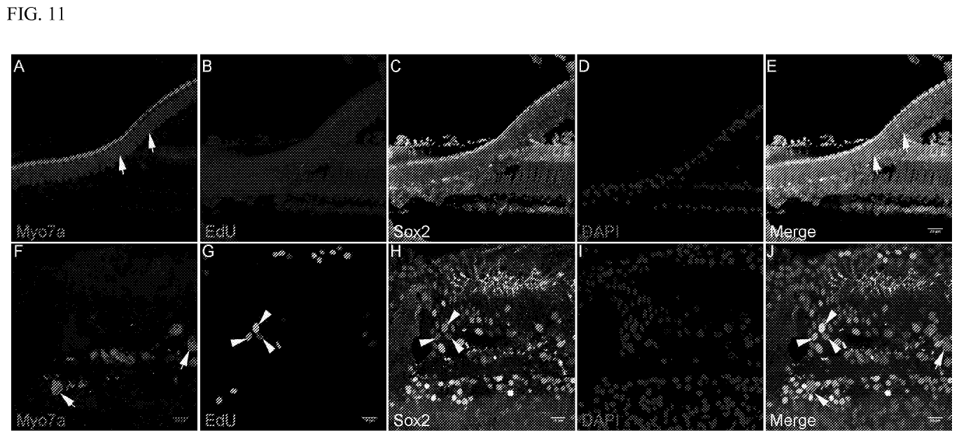

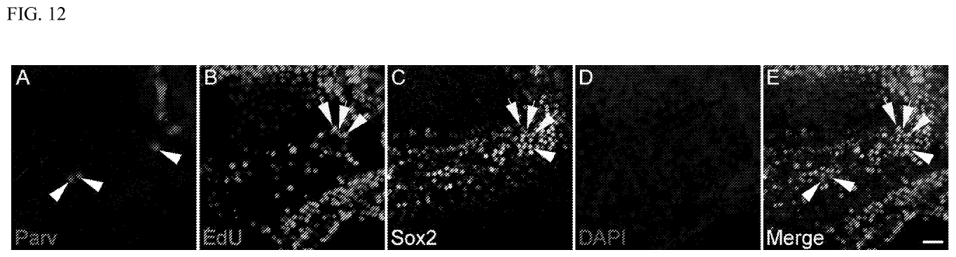

Additionally, experiments were performed in cultured cochlea harvested from adult monkeys. The culture medium contained DMEM/F12 supplied with N2 and B27 without serum. Cultured cochlea were exposed to an Ad-Myc/Ad-NICD mixture (final titer of 10⁹) for 16 hours, and the medium was replaced with fresh medium for 4 days. EdU was added at the final concentration of 10 M. Cycling cells were additionally labeled via EdU administration. Cultured cochlea were fixed and stained for hair and supporting cell markers, as well as EdU. Cycling Sox2+/EdU+ supporting cells were observed following exposure to elevated levels of c-Myc and NICD (FIG. 11, panels G, H, and J; arrowheads). Thus, this example demonstrates that cells of the monkey inner ear can also be induced to proliferate following exposure to elevated levels of c-Myc and Notch activity, suggesting that the disclosed method can be applied to mammals other than mice, e.g., primates. In cultured control monkey cochlea infected with Ad-Cre in the presence of EdU, no EdU labeled cells were seen (FIG. 11, panels A-E), a demonstration that no cells underwent proliferation. It is generally observed, both in cultured mouse and monkey cochlea that surviving inner hair cells rarely re-entered cell cycle, in contrast to mouse cochlea in vivo, in which inner hair cells could readily be induced to proliferation by the combination of c-Myc and NICD. It is likely that inner hair cells require a higher concentration of Myc and NICD and more time to proliferate, as the titer used in culture was not as high as in vivo (109 vs. 10¹²) and the tissues were harvested within a short period of time after infection (4 days).

### Example 4: Dose-Dependent Induction of Cell Proliferation in Cochlear Cell Subpopulations

The following example illustrates that different populations of cochlear hair cells are induced to proliferate upon varying degrees of exposure to c-myc and Notch activity.

An osmotic pump (Alzet) was implanted in the back of adult (45-day-old) doxycycline-inducible mice (rtTa/tet-on-Myc/tet-on-NICD) with tubing inserted to the round window niche to continuously dispense doxycycline (150 mg/ml in DMSO) at a rate of 1 □1 per hour for 9 days, with concurrent EdU administration (200 g/g body weight) by ip injection once daily to label proliferating cells. Using this procedure, c-Myc and NICD were activated in all cochlear cell types including supporting cells and hair cells (data not shown). Due to the surgical procedure, the cochlea in this sample lost all outer hair cells with only supporting cells and some inner hair cells remaining. Exposure of cochlear cells to this level of c-myc and NICD resulted in proliferation of Sox2+ supporting cells (FIG. 12, panels B, C, and E; arrows). By contrast Parv+ inner hair cells did not appear to divide upon exposure to these levels of c-myc and NICD (FIG. 12, panels A and E; arrowheads).

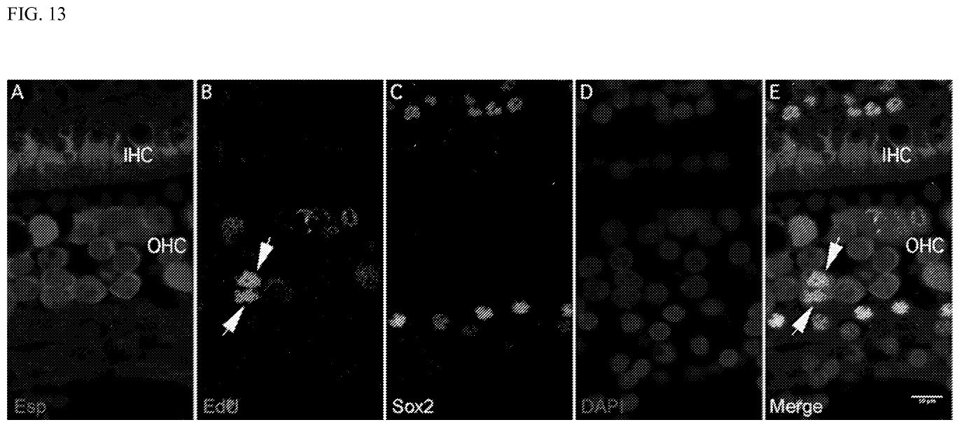

Additionally, the rTta/Tet-on-myc/Tet-on-NICD mouse model was used to examine induction of proliferation in outer hair cells. rTta/Tet-on-myc/Tet-on-NICD mice were exposed to doxycycline exposure for 12 days, accompanied by EdU administration once daily during the 12day period to label cycling cells, following the same procedure described for FIG. 12. Tissue was then harvested and stained for markers of hair cells (Esp) and supporting cells (Sox2). In this case, EdU+/Esp+ proliferating outer hair cells were observed following tissue harvest and staining (FIG. 13, panels A, B, and E; arrows). No cell proliferation was observed in inner hair cells. As this method activates c-Myc and NICD in all cochlear cell types, this example demonstrates that exposure of outer hair cells to elevated c-Myc and Notch activity can selectively induce outer hair cell cycle reentry and proliferation. In the same cochlea, fewer supporting cells (compared to outer hair cells) labeled with EdU were also seen (data not shown), which is consistent with the observation that outer hair cells have a greater capacity for cell cycle re-entry following c-Myc and NICD activation. This sample (FIG. 13) contrasts with the sample shown in FIG. 12 in that most of the outer hair cells survived and showed heightened proliferation capacity. It further indicates that after loss of outer hair cells, supporting cells can be induced to proliferate upon c-Myc and NICD activation (FIG. 12).

Taken together, these results indicate that while all populations of cochlear hair and supporting cells can be induced to differentiate upon exposure to elevated levels of c-myc and Notch activity, different subpopulations within the cochlea respond to different levels of c-myc and Notch exposure. For example, outer hair cells respond to lower levels of c-myc and Notch stimulation than supporting cells and inner hair cells. Supporting cells respond to lower levels of c-myc and Notch stimulation than inner hair cells, but require higher levels of c-myc and Notch stimulation than outer hair cells. Inner hair cells appear to require higher levels of c-myc and Notch stimulation than supporting cells and outer hair cells to promote cell proliferation.

### Example 5: Functional Characteristics of Hair Cells Produced by Myc and Notch Exposure

The following examples demonstrate that hair cells produced by applying the methods described herein possess characteristics of functional hair cells.

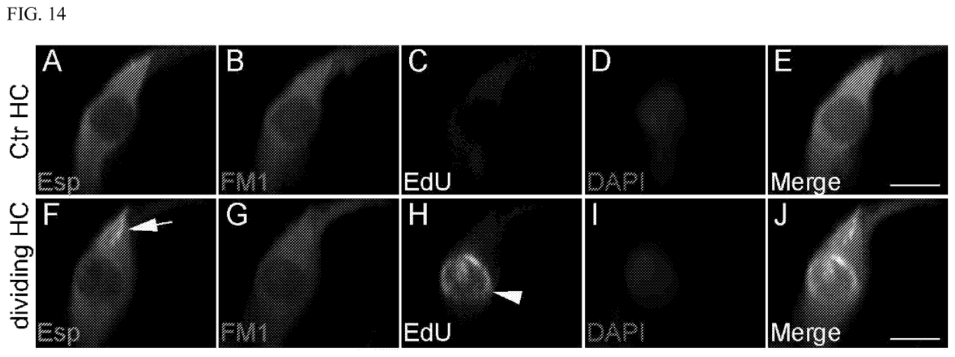

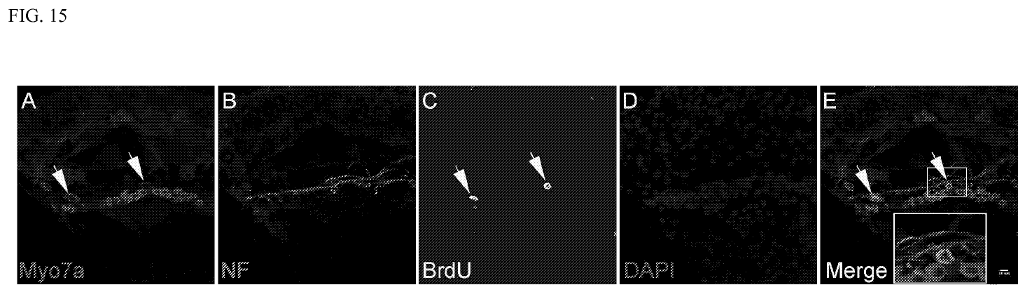

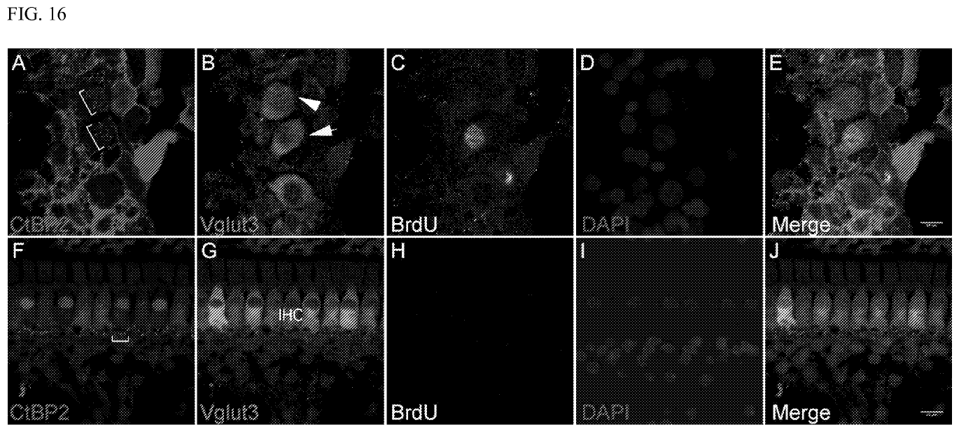

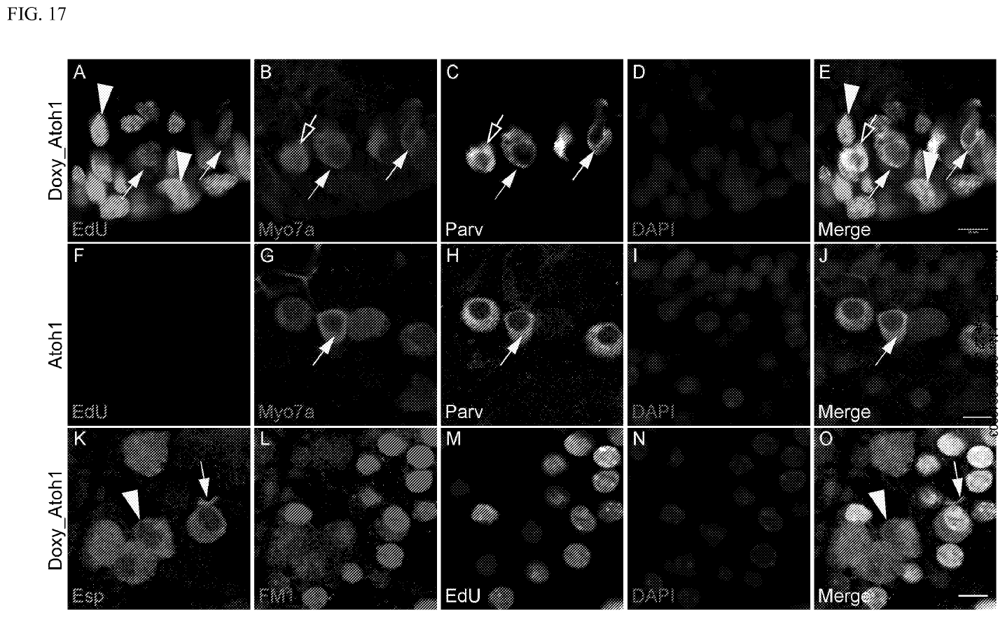

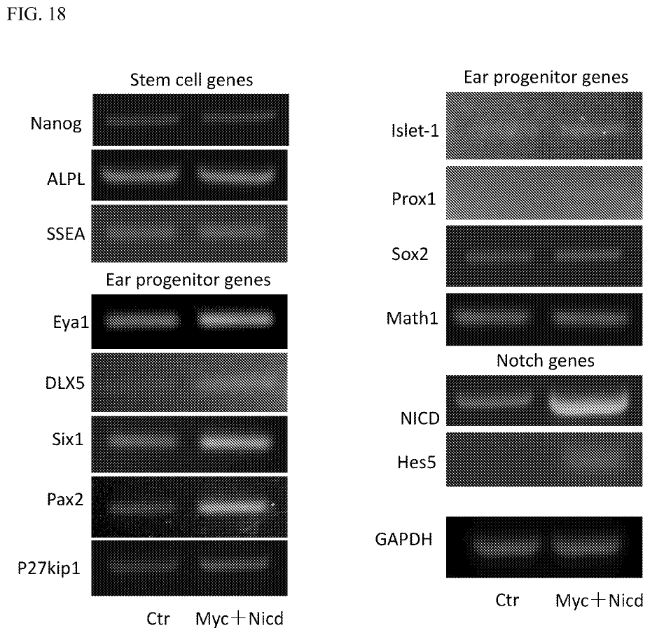

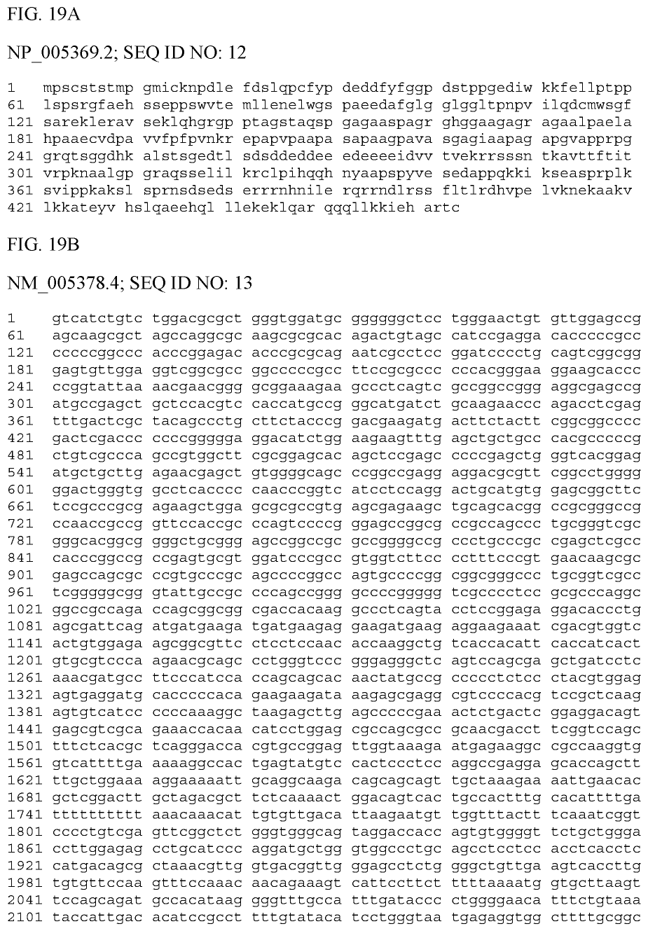

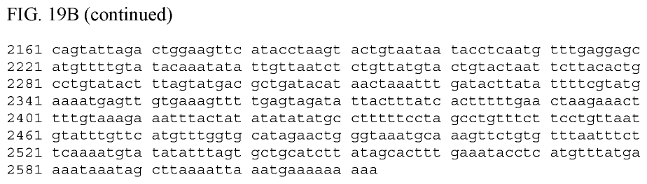

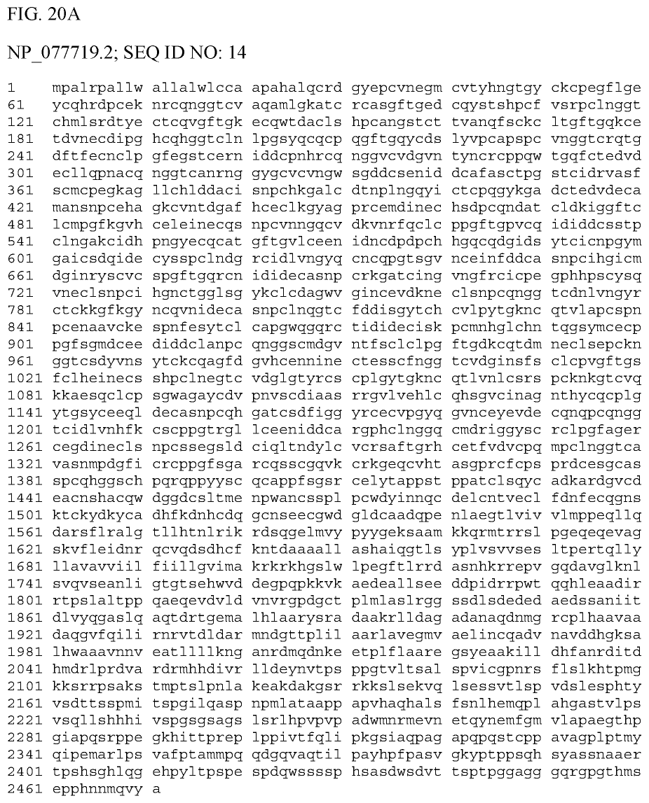

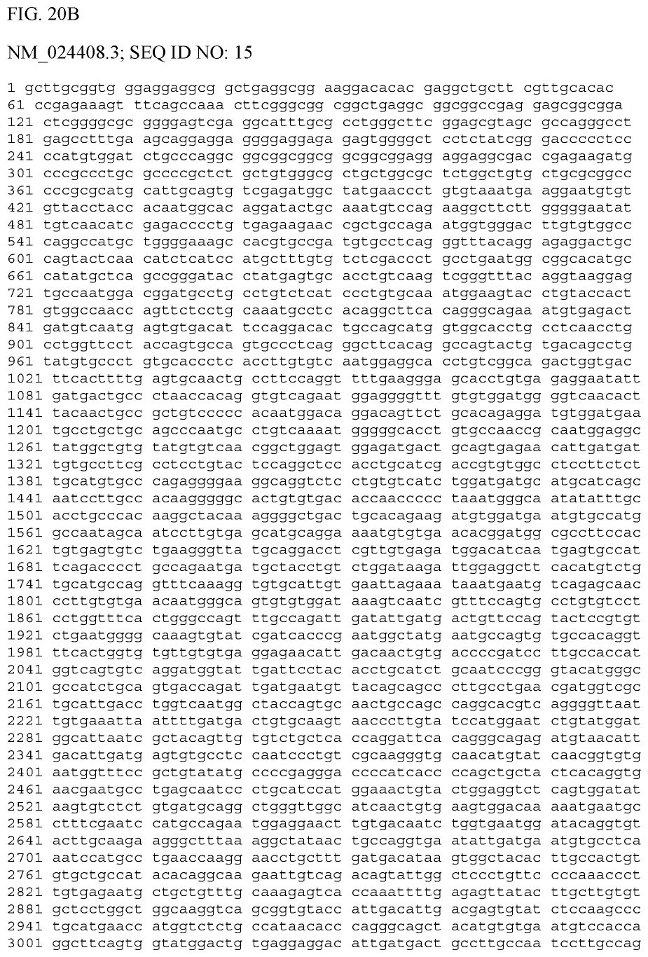

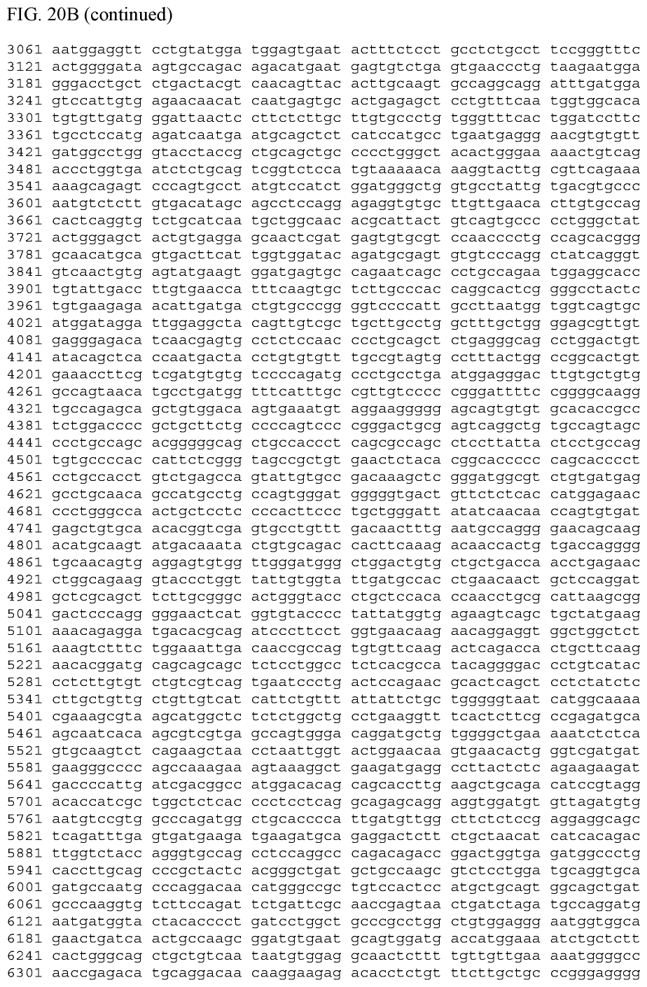

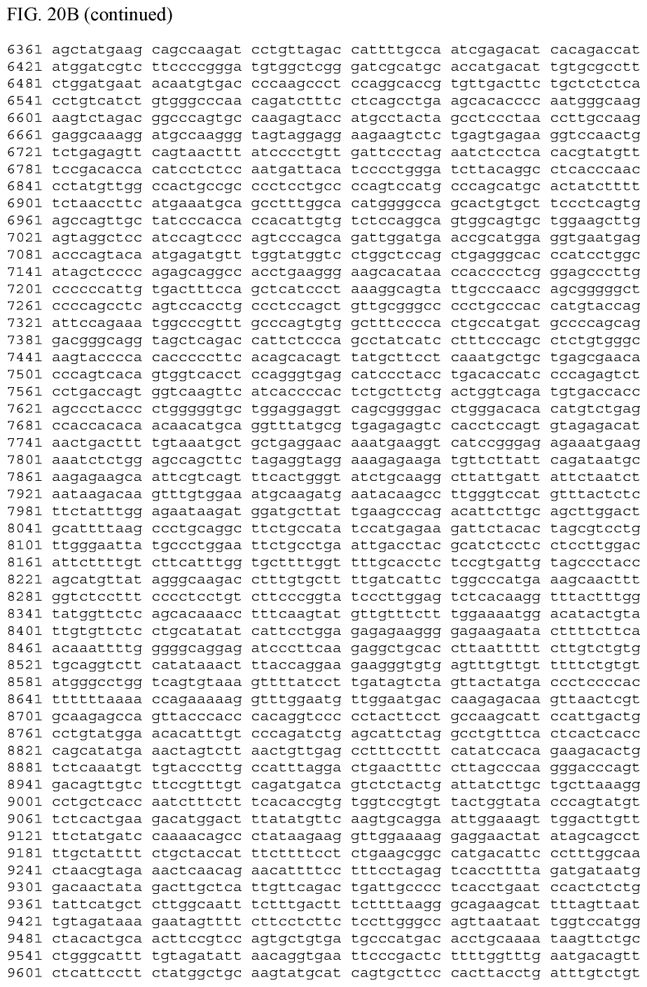

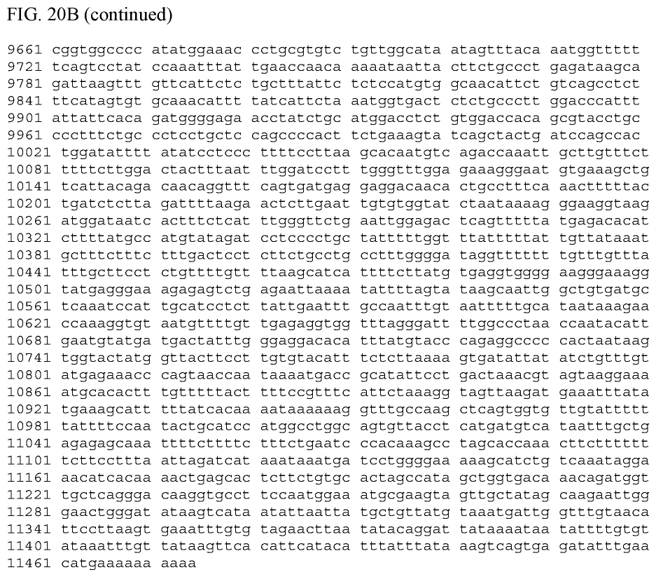

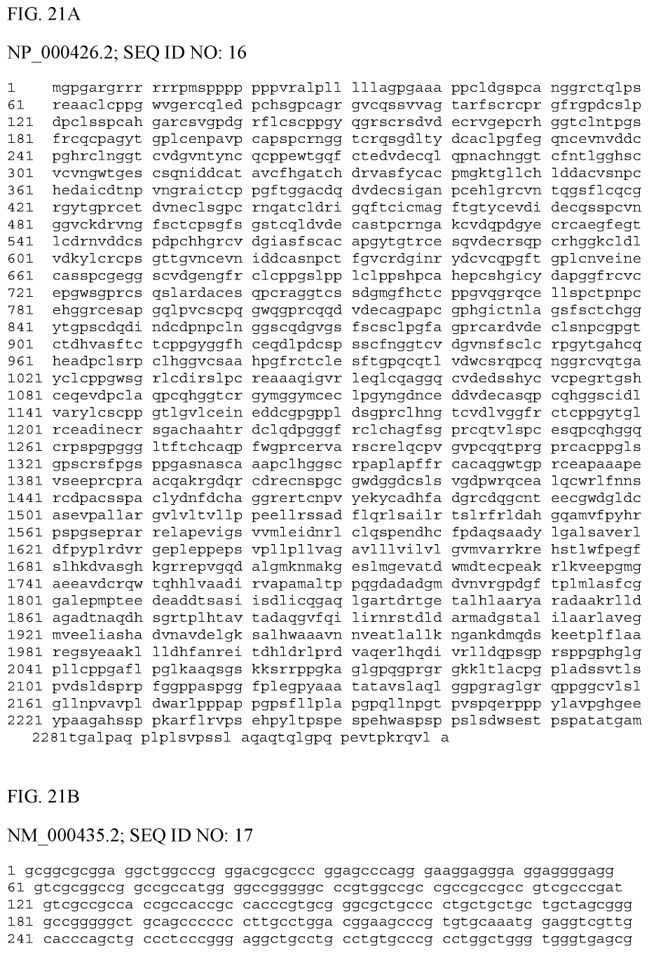

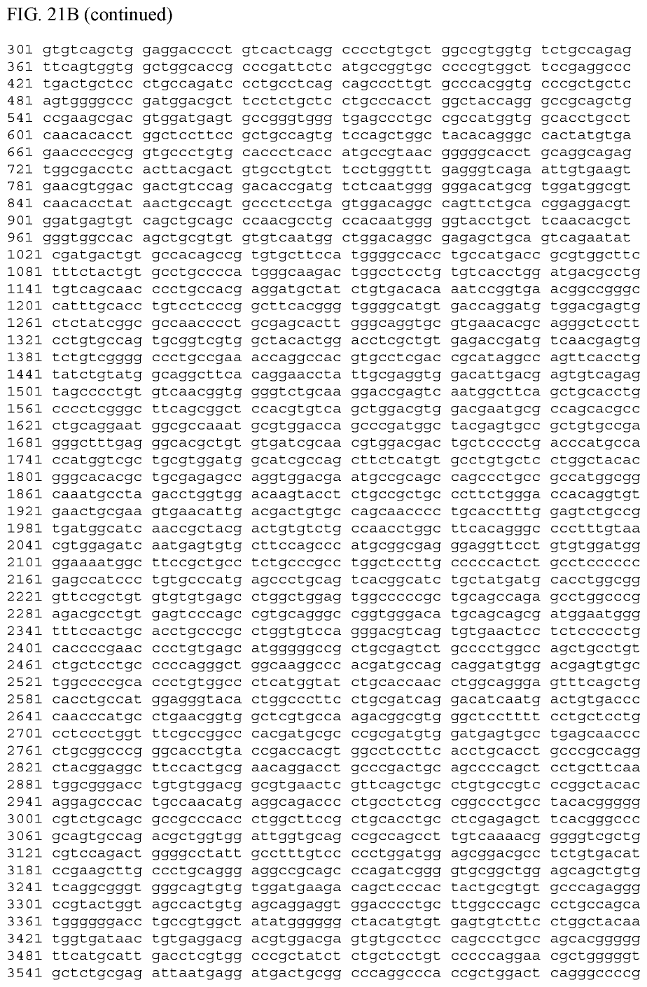

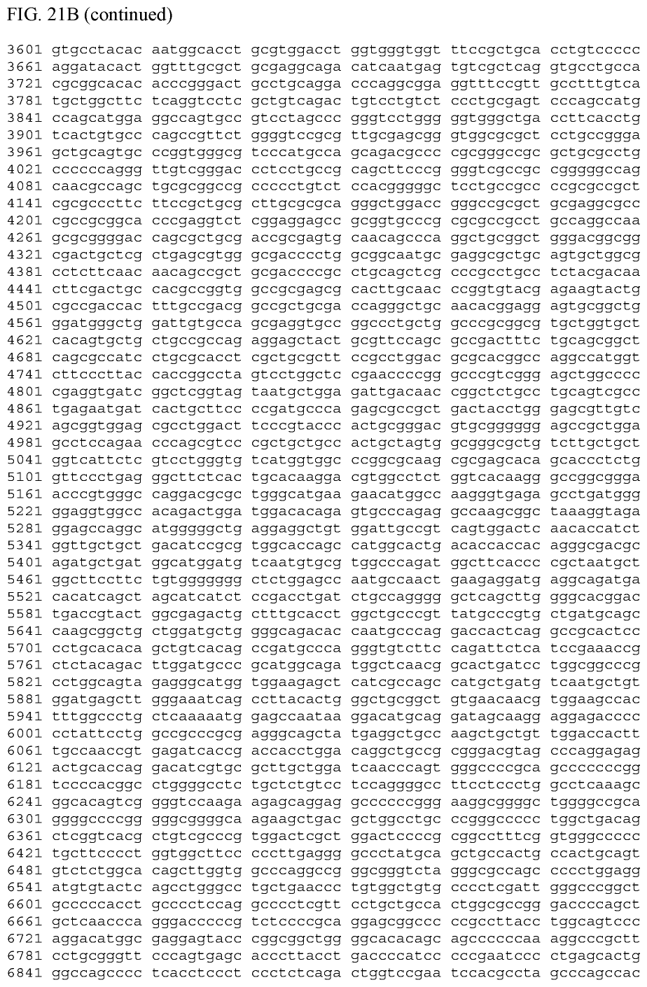

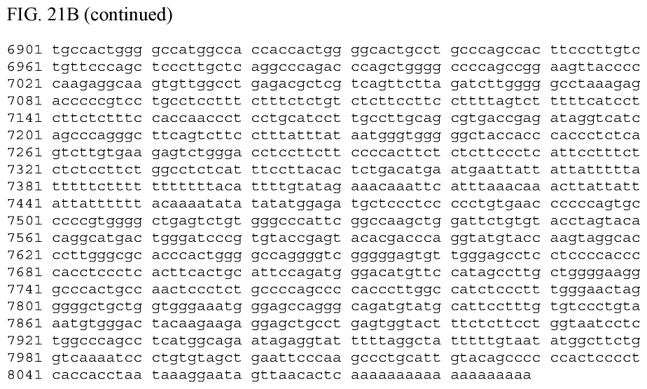

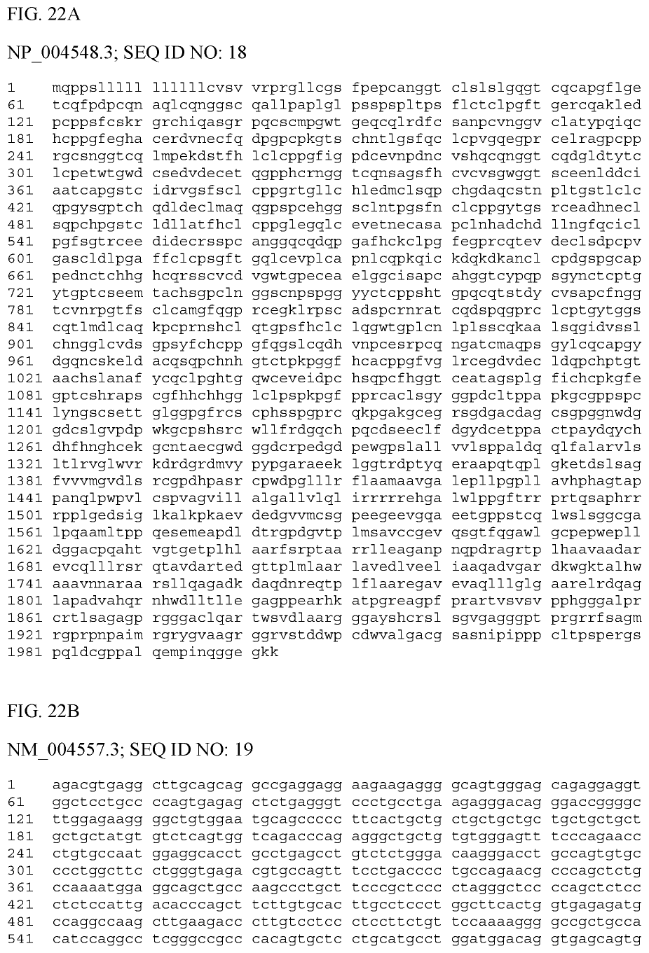

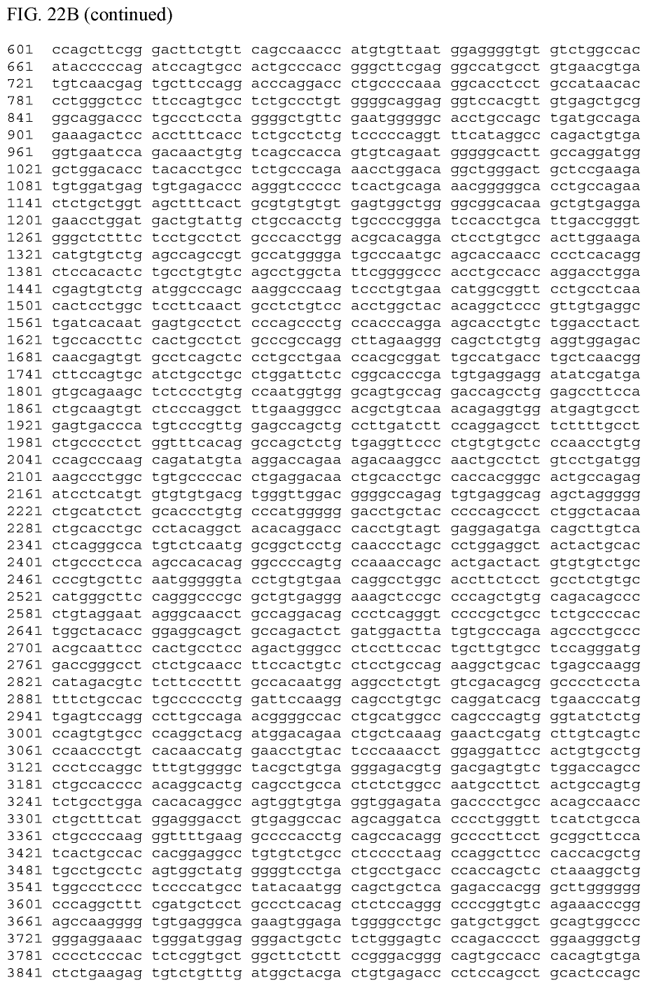

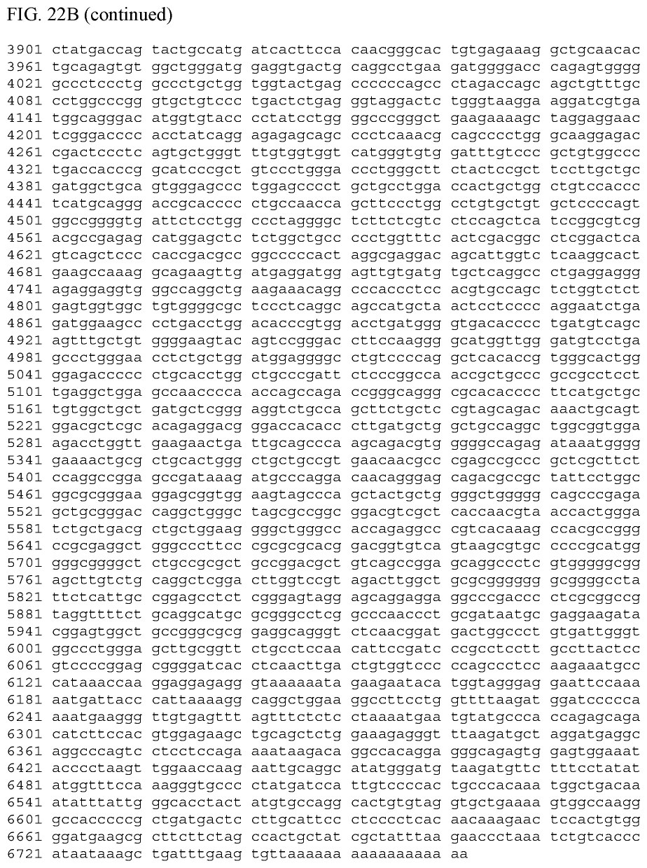

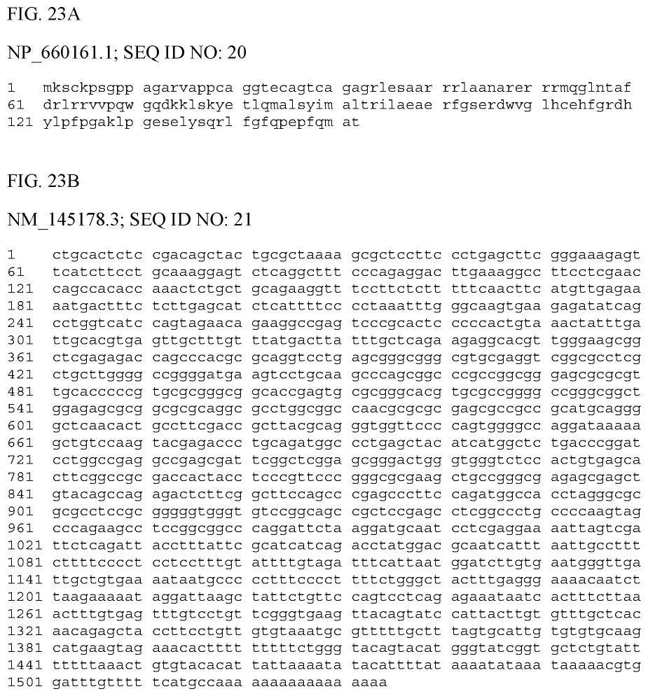

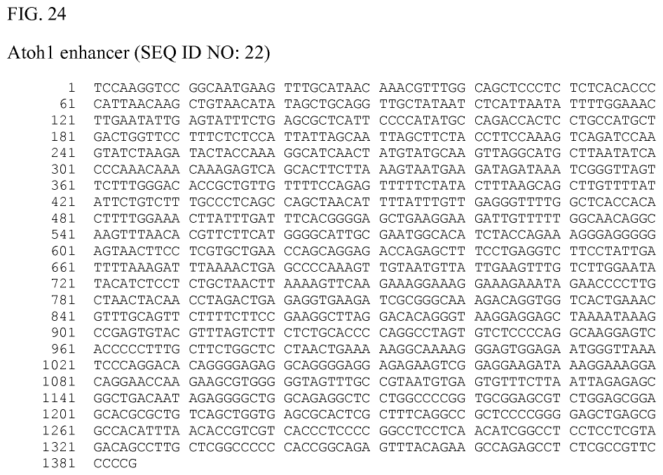

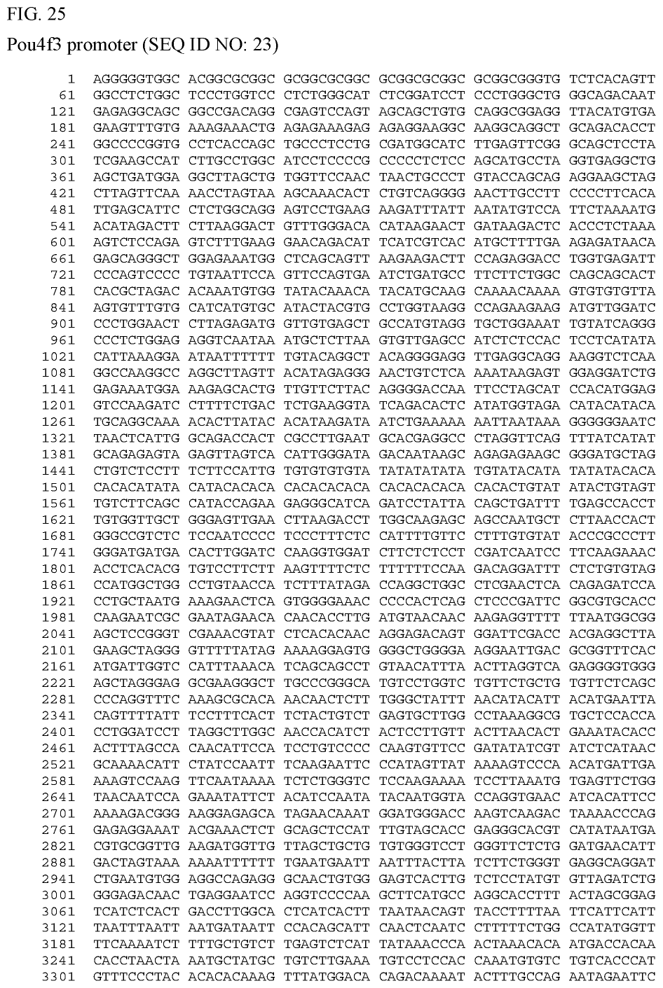

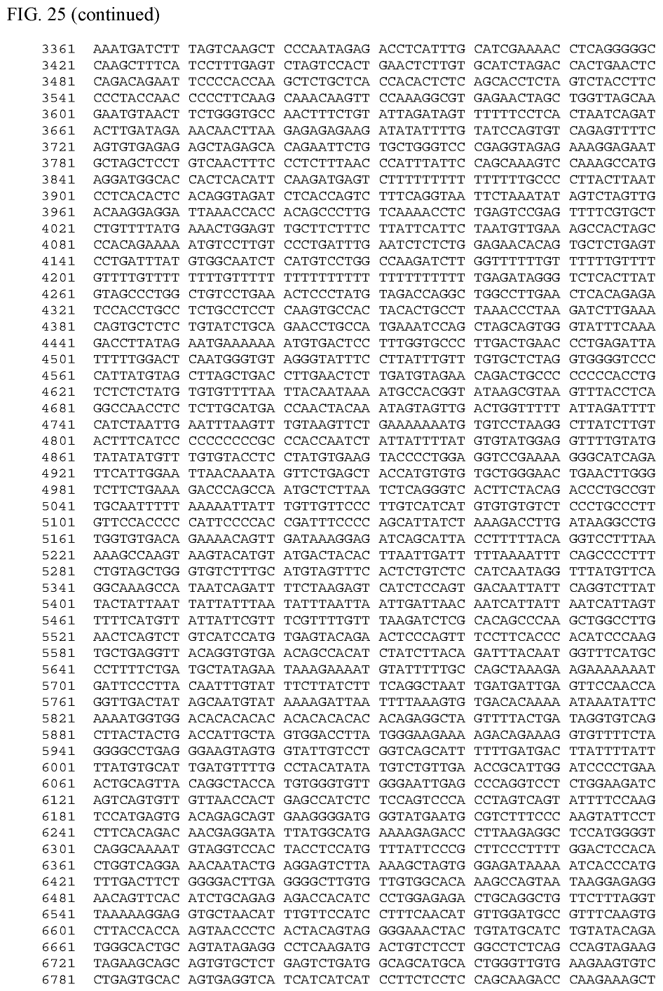

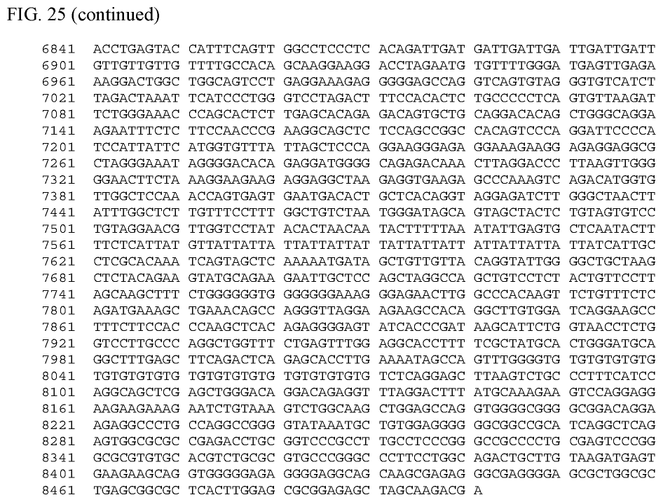

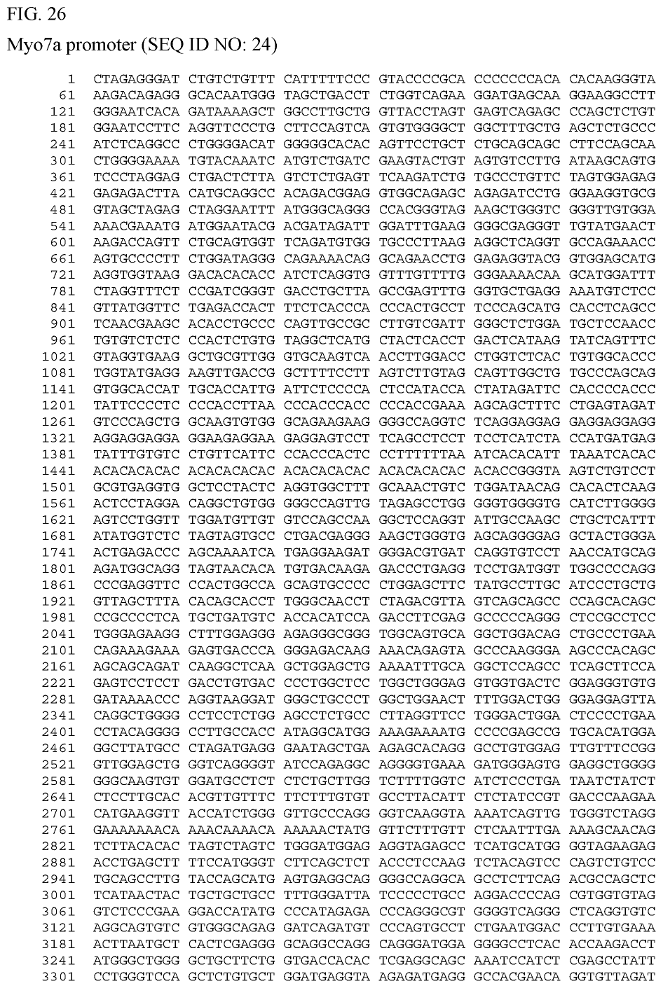

The presence of signal transduction channels necessary for hair cell function was assessed in hair cells produced by elevated Myc and Notch exposure. 45-day-old NICDflox/flox mice were injected with Ad-Cre-GFP and Ad-Myc mixture in the scala media using cochleostomy. EdU was injected for 5 days daily following adenovirus injection to label proliferating hair cells. 35 days post-virus injection, mouse cochleas were dissected and incubated with fluorescence dye FM1-43FX for 30 seconds before cochleas were washed and fixed. Fixed tissues were decalcified and stained with Espin (Esp) for hair cells. Cells that underwent proliferation were labeled by EdU. FIG. 14 shows that control Esp+ hair cells that did not undergo cell cycle reentry following EdU exposure (EdU-) took up FM1-43FX (FIG. 14, panels A-E). Significantly, Esp+ hair cells that reenter the cell cycle following Ad-Myc/Ad-NICD virus injection and EdU exposure (EdU+) also took up FM1-43FX (FIG. 14, panels F-J). As FM1-43FX rapidly enters hair cells through functional transduction channels, labeling by FM1-43FX demonstrates the presence of functional transduction channels in proliferating hair cells similar to non-proliferating hair cells. This result demonstrates that hair cells produced by exposure to elevated Myc and Notch activity possess functional membrane channels that are essential for hair cell function.

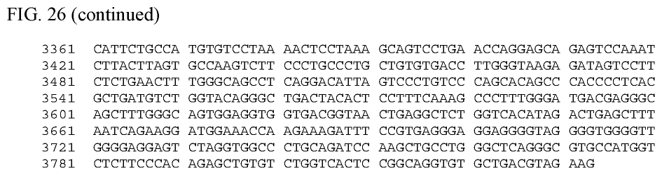

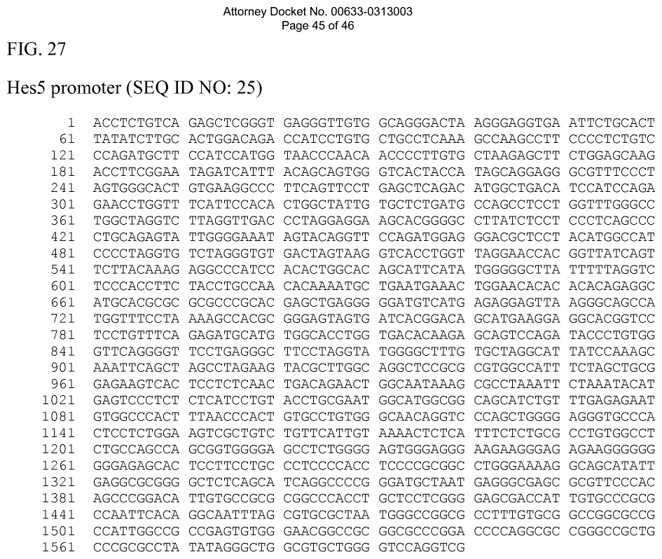

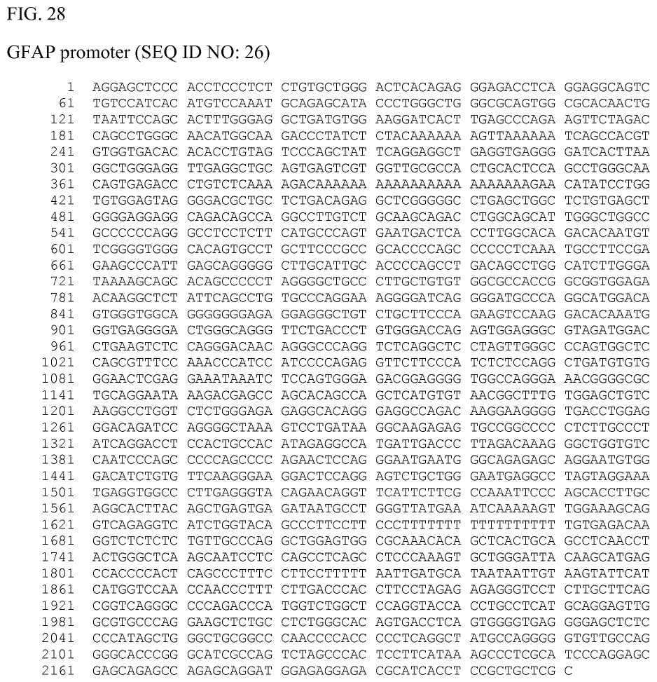

Synapse formation was also assessed in cells exposed to elevated levels of c-Myc and Notch activity in vivo. Adult (45-day-old) NICD^{flox/flox} mice were transduced with an Ad-Myc/Ad-Cre virus mixture, exposed to BrdU administration, and analyzed for evidence of functional synapse formation as described for FIG. 9. Tissue was harvested 20 days post-injection of virus and stained for neurofilament (NF) to identify neurofibers of ganglion neurons. Analysis of stained sections revealed the presence of proliferating hair cells (Myo7a+/BrdU+) that were in contact with NF+ neurofibers (FIG. 15, panels A, C, and E; arrows). This result suggests that production of hair cells via the methods disclosed herein is accompanied by regrowth of neurofibers and formation of functional synapses crucial for hair cell function.

### Example 6: Hair Cells Induced to Proliferate In Vivo Maintain Specific Hair Cell Identity

The following example illustrates that inner hair cells produced in vivo via induced proliferation of existing inner hair cells maintain characteristics specific to inner hair cells.

Cochlea of adult NICD^{flox/flox}mice were transduced in vivo with an Ad-Myc/Ad-Cre virus mixture for 15 days with BrdU injected daily for the first 5 days. The methods used are the same as those described for FIG. 9. Cochlear tissue was harvested and analyzed for inner hair cell-specific markers. Both inner hair cells that underwent cell cycle reentry (FIG. 16, panels A-E; arrow) and those that did not undergo cell cycle reentry (FIG. 16, panels A-E; arrowhead) stained positive for Vesicular Glutamate Transporter-3 (Vglut3), an inner hair cell-specific marker.

Furthermore, the same cells also stained positive for C-Terminal Binding Protein 2 (CtBP2) (brackets), a presynaptic marker, indicating the presence of functional synapses. By contrast, in control animals exposed to Ad-GFP, no BrdU labeling was observed, although Vglut3+/CtBP2+ inner hair cells were detected (FIG. 16, panels F-J, bracket). The results show that induced proliferation of inner hair cells via exposure to elevated c-myc and Notch activity produce inner hair cells with markers of functional synapses.

### Example 7: Transdifferentiation of Proliferating Supporting Cells in Culture

The following example demonstrates that application of the methods described herein can be used to induce proliferation and transdifferentiation of inner car support cells to a hair cell fate.

Experiments were performed using a mouse model capable of expressing elevated levels of myc and Notch following doxycycline induction (rTta/Tet-on-Myc/Tet-on-NICD). Adult mouse (rTta/Tet-on-Myc/Tet-on-NICD) cochlea was dissected, with three holes drilled to the bone for efficient media exposure and cultured in the DMEM/F12 supplied with N2 and B27 without scrum. Doxycycline (1 mg/ml) was added to the culture for 5 days to activate c-Myc/NICD, followed by Ad-Atoh1 (2×10¹², 1:100 dilution) infection for 16 hours. The culture was exchanged with fresh medium for additional 14 days, with medium changed every 3 days. EdU (final concentration 10 M) was added to the culture throughout the entire period. Support cells induced to express elevated NICD and myc levels via doxycycline exposure were observed to undergo cell proliferation as evidenced by EdU labeling (FIG. 17, panels A-E, arrowheads and closed arrows). Furthermore, exposure to Ad-Atoh1 resulted in transdifferentiation of both cycling (FIG. 17, panels A, C, E, closed arrows) and non-cycling (FIG. 17, panels B, C, E, open arrow) support cells to a hair cell fate as evidenced by Myo7a and Parvalbumin (Parv) staining. Control, cultured rTta/Tet-on-Myc/Tet-on-NICD support cells exposed to Ad-Atoh1, but not doxycycline, underwent transdifferentiation but failed to undergo cell cycle reentry (FIG. 17, panels F-J, arrow), as evidenced by the presence of Myo7a+/Parv+/EdU-cells. In a similar experiment, cultured cochlear supporting cells harvested from rTta/Tet-on-Myc/Tet-on-NICD mice were exposed to doxycycline and Ad-Atoh1 virus, and then exposed to FM1-43FX (3 M) for 30 seconds to investigate whether hair cells produced by this process possess characteristics of functional hair cells. Esp staining of cells subjected to this protocol revealed the presence of hair bundles in transdifferentiated supporting cells that also stained positive for FMI uptake, revealing the presence of functional membrane channels (FIG. 17, panels K and O; arrow). Other transdifferentiated cells were labeled with FM1, but did not show signs of cell cycle reentry as they are EdU negative (FIG. 17, panels K and O; arrowhead). Thus, exposure of cultured cochlear support cells to elevated levels of myc and Notch, followed by Atoh1 induced proliferation of supporting cells and transdifferentiation to a hair cell fate, where the cells generated possessed characteristics of functional hair cells.

### Example 8: Induction of Inner Ear Progenitor Gene Expression

In order to understand how cell fate is affected by elevated c-myc and Notch activity, a study of mRNA transcripts expressed following exposure to c-Myc and NICD was performed.

Adult NICD^{flox/flox} mouse cochleas were cultured and infected with Ad-Myc/Ad-Cre-GFP overnight (2×10¹² in 1:100 dilution). Beginning the next day, the media was changed daily for the next 4 days. Ad-Cre-GFP infected NICD^{flox/flox} mouse cochleas were used as controls. The infected cochleas were harvested for mRNA isolation using QIAGEN mRNA isolation kit. cDNAs were synthesized using Life Science Technology SuperScript III reverse transcriptase kit. Semi-quantitative RT-PCR was performed using standard protocol. Analysis of different sets of transcripts revealed that stem cell gene transcripts (e.g., Nanog, ALPL, SSEA) were not noticeably upregulated following c-myc and NICD exposure. By contrast, most of the analyzed transcripts specific to car progenitor cells (e.g., Eyal, DLX5, Six1, Pax2, p27kip1, NICD, Prox1, Hes5) were upregulated following exposure to c-myc and NICD (FIG. 18). GAPDH served as an internal control for normalization of signal intensity. These results suggest a decisive advantage inherent in using the method disclosed herein, as opposed to using embryonic stem cells. Specifically, these results demonstrate that exposure to elevated c-Myc and Notch activity results in elevated levels of progenitor, rather than stem cell gene expression, which likely allows the inner car cells to both re-enter the cell cycle and maintain the desired cell fate.

### INCORPORATION BY REFERENCE

The entire disclosure of each of the patent documents and scientific articles cited herein are incorporated by reference in their entirety for all purposes.

### EQUIVALENTS

The invention can be embodied in other specific forms with departing from the essential characteristics thereof. The foregoing embodiments therefore are to be considered illustrative rather than limiting on the invention described herein. The scope of the invention is indicated by the appended claims rather than by the foregoing description, and all changes that come within the meaning and range of equivalency of the claims are intended to be embraced therein.

1-17. (canceled)

18. A method of inducing proliferation and transdifferentiation of a supporting cell in the inner ear of a subject, the method comprising:
increasing both c-myc activity and Notch activity within the supporting cell; and
increasing Atoh1 activity after proliferation of the supporting cell.

19. The method of claim 18, wherein c-myc activity is increased by administering an effective amount of a composition comprising: a c-myc protein, a nucleic acid encoding a c-myc protein, a viral vector comprising a nucleic acid encoding a c-myc protein, a lipid particle comprising a nucleic acid encoding a c-myc protein, or a c-myc activator.

20. The method of claim 19, wherein the composition comprises a viral vector comprising a nucleic acid encoding a c-myc protein, wherein the viral vector is selected from the group consisting of an adenoviral vector, a lentiviral vector, a retroviral vector, and an adeno-associated viral vector.

21. The method of claim 19, wherein the composition comprises a nucleic acid encoding a c-myc protein.

22. The method of claim 19, wherein the c-myc protein comprises the amino acid sequence of SEQ ID NO: 1.

23. The method of claim 19, wherein the composition comprises a c-myc activator, wherein the c-myc activator is a microRNA that targets FBXW-7.

24. The method of claim 19, wherein the composition is administered to the inner ear of the subject.

25. The method of claim 18, wherein Notch activity is increased by administering an effective amount of a composition comprising: a Notch protein, a Notch Intracellular Domain (NICD) protein, a nucleic acid encoding a Notch protein, a nucleic acid encoding an NICD protein, a viral vector comprising a nucleic acid encoding a Notch protein or an NICD protein, a lipid particle comprising a nucleic acid encoding a Notch protein or an NICD protein, or a Notch activator.

26. The method of claim 25, wherein the composition comprises a viral vector comprising a nucleic acid encoding an NICD protein, wherein the viral vector is selected from the group consisting of an adenoviral vector, a lentiviral vector, a retroviral vector, and an adeno-associated viral vector.

27. The method of claim 25, wherein the composition comprises a nucleic acid encoding an NICD protein.

28. The method of claim 25, wherein the NICD protein comprises the amino acid sequence of SEQ ID NO: 7.

29. The method of claim 25, wherein the composition comprises a Notch activator, wherein the Notch activator is selected from the group consisting of: a vector comprising a nucleic acid encoding a Jagged protein, a Serrate protein, or a Delta protein; valproic acid;
resveratrol; and phenethyl isothiocyanate.

30. The method of claim 25, wherein the composition is administered to the inner ear of the subject.

31. The method of claim 18, wherein c-myc activity and Notch activity are increased by administering an effective amount of composition comprising a viral vector, wherein the viral vector comprises a nucleic acid encoding a c-myc protein and a nucleic acid encoding an NICD protein.

32. The method of claim 18, wherein Atoh1 activity is increased by administering an effective amount of a composition comprising: an Atoh1 protein, a nucleic acid encoding an Atoh1 protein, a viral vector comprising a nucleic acid encoding an Atoh1 protein, a lipid particle comprising a nucleic acid encoding an Atoh1 protein, or an Atoh1 activator.

33. The method of claim 32, wherein the composition comprises a viral vector comprising a nucleic acid encoding an Atoh1 protein, and wherein the viral vector is selected from the group consisting of an adenoviral vector, a lentiviral vector, a retroviral vector, and an adeno-associated viral vector.

34. The method of claim 32, wherein the nucleic acid encoding the Atoh1 protein is operably linked to a promoter sequence.

35. The method of claim 32, wherein the Atoh1 protein comprises the amino acid sequence of SEQ ID NO: 3.

36. The method of claim 32, wherein the composition comprises an Athoh1 activator, wherein the Atoh1 activator is selected from the group consisting of: a β-Catenin protein; a Wnt ligand; a DVL protein; an LRP6 protein; a Wnt protein; a nucleic acid encoding a β-Catenin protein, a DVL protein, an LRP6 protein, or a Wnt protein; and lithium chloride.

37. The method of claim 32, wherein the composition is administered to the inner ear of the subject.
